(19) United States
(12) Patent Application Publication
(10) Pub. No.: US 2015/0082480 A1
(43) Pub. Date: Mar. 19, 2015

(54) Methods to alter plant cell wall composition for improved biofuel production and silage digestibility

(71) Applicants: PIONEER HI BRED INTERNATIONAL INC, JOHNSTON, IA (US); E I DU PONT DE NEMOURS AND COMPANY, WILMINGTON, DE (US)

(72) Inventors: Kanwarpal Dhugga, Johnston, IA (US); David Dolde, Johnston, IA (US); Rajeev Gupta, Johnston, IA (US); Ajay Pal Singh Sandhu, Wilmington, DE (US); Carl Simmons, Des Moines, IA (US)

(21) Appl. No.: 14/353,367

(22) PCT Filed: Oct. 24, 2012
(86) PCT No.: PCT/US2012/061539

§ 371 Date: Apr. 22, 2014

Related U.S. Application Data
(60) Provisional application No. 61/551,067, filed on Oct. 25, 2011.

(51) Int. Cl. C12N 15/82; C07C 31/08

(52) U.S. Cl. CPC C12N 15/8243; C07C 31/08; C12N 15/8245; C12N 15/8261
USPC 800/278; 435/419; 568/840; 800/284; 800/290; 800/298; 800/312; 800/314; 800/320; 800/320.1; 800/320.2; 800/320.3; 800/322

(57) ABSTRACT

The disclosure provides means for altering the expression of non-cellulosic polysaccharides in plants using Golgi targeted enzyme nucleic acids and their encoded proteins. The present disclosure provides methods and compositions relating to altering feruloylation, acetylation and crosslinking in plants, leading to improved biomass available for biofuel production and silage digestibility. The disclosure further provides recombinant expression cassettes, host cells, and transgenic plants comprising said nucleic acids.

### TECHNICAL FIELD

The present disclosure relates generally to plant biochemistry and molecular biology. More specifically, it relates to enzymes, butanol, ethanol, nucleic acids and methods for modulating their presence in plants.

### BACKGROUND

Ethanol production in the US used approximately 37% of the total corn crop in 2010. As global demand for food increases because of increasing population, it is imperative to explore other feedstock sources than grain for ethanol production. After the grain is harvested, the crop residue, referred to as stover, is left in the field. The proportion of stover in a corn plant is approximately the same as grain, and ⅔^{rd} of the stover may be removed without significantly affecting the soil organic matter content (Dhugga, (2007) Crop Sci. 47:2211-2227; Graham, et al., (2007) Agronomy Journal 99:1-11; Johnson, et al., (2006) Journal of Soil and Water Conservation 61:120A-125A; Perlack, et al., (2005) Biomass as Feedstock for a Bioenergy and Bioproducts Industry: The Technical Feasibility of a Billion-Ton Annual Supply. U.S. Department of Energy, Oak Ridge, Tenn.; Wilhelm, et al., (2004) Agronomy Journal 96:1-17). Once production of sugars from the crop residue is streamlined, corn stover alone can contribute substantially toward ethanol production.

Butanol is the preferred form of alcohol as a biofuel because of its lower oxygen to carbon ratio as well as its ability to keep water out. Ethanol absorbs water, which contributes to the corrosion of the supply pipeline, a problem butanol could overcome. Transportation of liquid fuels through a pipeline is more economical than via railcars. Crop residue could be looked upon essentially as a sugar platform that could be used to produce either of these alcohols depending upon which technology is more efficient (Dhugga, 2007). Nearly all the crop residue is made of cell walls, which consist of cellulose microfibrils embedded in a matrix of hemicellulose and lignin. Small amounts of proteins and minerals are also present. Hemicellulose in grasses consists primarily of glucuronoarabinoxylan (GAX), a xylan backbone that carries arabinosyl and glucuronosyl residues as side groups (Carpita, (1996) Annual Review Of Plant Physiology And Plant Molecular Biology 47:445-476). In addition, acetyl groups are esterified at 2^{nd} and 3^{rd} carbons of the xylosyl residues. Approximately, ½ to ⅓ of all the xylosyl residues in GAX are acetylated in maize, however, acetate content varies across species (Dhugga, 2007). Arabinosyl residues in GAX become feruloylated in the Golgi apparatus.

Ethanol production from corn stover has not yet become commercially profitable because mainly of two bottlenecks in the process, that is, pretreatment cost and fermentation efficiency. Pretreatment is used to loosen the cell wall and is believed to break lignin-lignin and lignin-polysaccharide cross-links, thereby increasing the accessibility of the carbohydrate fraction of the wall to the hydrolytic enzymes (Dhugga, 2007). Reduction in lignin through genetic selection or engineering almost invariably leads to a reduction in biomass production (Pedersen, et al., (2005) Crop Science 45:812-819). This disclosure shows that it is possible to reduce ferulate content of the wall without an adverse effect on plant biomass.

Acetate is a known inhibitor of fermentation both in Zymomonas and yeast (Franden, et al., (2009) Journal of Biotechnology 144:244-259; Ho, et al., (1999) “Successful Design and Development of Genetically Engineered Saccharomyces Yeasts for Effective Cofermentation of Glucose and Xylose from Cellulosic Biomass to Fuel Ethanol” Advances in Biotechnology/Engineering Vol. 45, Ed. Th. Scheper, Springer-Verlag, Berlin Heidelberg). With a trend in ethanol industry toward simultaneous saccharification and fermentation (SSF), acetate stays in the processing tank after biomass pre-treatment and thus interferes with fermentation.

The hemicellulosic polysaccharides are first made in the Golgi and then exported to the cell wall by exocytosis (Northcote and Pickett-Heaps, (1966) Biochemical Journal 98:159-167; Ray, et al., (1976) Ber. Deutsch. Bot. Ges. Bd. 89:121-146). Although a number of genes that affect xylan content of the wall have been identified through mutational genetics, the exact mechanism of GAX biosynthesis remains thus far elusive, making it a challenge to alter wall composition through affecting the Golgi biosynthetic machinery (Scheller and Ulvskov, (2010) Hemicelluloses. Annual Review of Plant Biology, pp 263-289).

Down-regulation of lignin through interference with the monolignol biosynthetic pathway has been accomplished in several commercial crop plants; however, this is accompanied by a reduction in biomass production. Improved digestibility of the altered biomass for silage or ethanol production is not sufficient to overcome the loss incurred by reduced biomass production (Dhugga, 2007; Pedersen, Vogel, and Funnell 2005). Previous attempts at cell wall remodeling through alteration of pectin structure in potato were successful (Skjøt, et al., 2002).

Down-regulation of the degree of feruloylation (and thus cross-linking) as well as acetyl content improves the quality of biomass for biofuels. Non-cellulosic wall polysaccharides are first synthesized in the Golgi and then exported to the cell wall through exocytosis. Interference with the biosynthesis of cell wall matrix polysaccharides by targeting hydrolases or esterases to the Golgi compartment could be another avenue to alter wall composition. Ectopic expression of esterases or glycosidases specific to various groups of complex polysaccharides in the Golgi apparatus leads to altered cell wall composition.

### SUMMARY

Generally, it is the object of the present disclosure to provide nucleic acids and proteins relating to non-cellulosic cell wall polysaccharides. It is an object of the present disclosure to provide transgenic plants comprising the nucleic acids of the present disclosure and methods for modulating, in a transgenic plant, expression of the nucleic acids of the present disclosure, in such a way as to modify acetate concentration in the plant.

Therefore, in one aspect the present disclosure relates to an isolated nucleic acid comprising a member selected from the group consisting of (a) a polynucleotide having a specified sequence identity to a polynucleotide encoding a polypeptide of the present disclosure; (b) a polynucleotide which is complementary to the polynucleotide of (a) and (c) a polynucleotide comprising a specified number of contiguous nucleotides from a polynucleotide of (a) or (b). The isolated nucleic acid can be DNA.

In other aspects the present disclosure relates to: 1) recombinant expression cassettes, comprising a nucleic acid of the present disclosure operably linked to a promoter, 2) a host cell into which has been introduced the recombinant expression cassette, 3) a transgenic plant comprising the recombinant expression cassette and 4) a transgenic plant comprising a recombinant expression cassette containing more than one nucleic acid of the present disclosure each operably linked to a promoter. Furthermore, the present disclosure also relates to combining by crossing and hybridization recombinant cassettes from different transformants. The host cell and plant are optionally from maize, wheat, rice, sugarcane, sunflower, grass or soybean.

In other aspects the present disclosure relates to methods of altering cell wall composition and physical traits, including, but not limited to crosslinking and improving biomass quality, through the introduction of one or more of the polynucleotides that encode the polypeptides of the present disclosure, which when expressed lead to reduced cell wall acetate content and altered sugar composition in the plant. Additional aspects of the present disclosure include methods and transgenic plants useful in the end use processing of non-cellulosic polysaccharides such as those produced in the Golgi or use of transgenic plants as end products either directly, such as silage, or indirectly following processing, for such uses known to those of skill in the art, such as, but not limited to, ethanol and other biofuels. Also, one of skill in the art would recognize that the polynucleotides and encoded polypeptides of the present disclosure can be introduced into a host cell or transgenic plant singly or in multiples, sometimes referred to in the art as “stacking” of sequences or traits. It is intended that these compositions and methods be encompassed in the present disclosure.

Additional methods include but are not limited to:

A method of reducing acetate and/or ferulate content in a plant, the method comprising expressing an enzyme that cleaves acetyl or feruloyl substituents and targeting the cleaving enzyme to one or more components of the Golgi apparatus or manipulating the endogenous enzyme. In addition this method, wherein the enzyme is an acetyl esterase or a feruloyl esterase. Also this method, wherein the plant biomass is not substantially reduced compared to a plant not expressing the esterase targeted to the Golgi. And the same method, wherein the enzyme targeted to Golgi is: an acetyl esterase, a feruloyl esterase, and/or an arabinosidase.

Also contemplated is the previous method comprising the steps of transforming a plant cell with a vector containing a polynucleotide encoding a heterologous esterase, targeting the expression of said enzyme to the Golgi apparatus, retaining expression of said hydrolytic enzyme in the Golgi apparatus and growing said plant under plant growing conditions. In addition to those method steps, the method which improves composition of the biomass of a plant by overexpression of the polynucleotide. Also this same method in which: ethanol production is improved, the transformed plant cell further comprises one or more heterologous polynucleotides encoding a hydrolase, esterase, glycosyltransferase or arabinofuranosidase, the transformed plant cell wall polysaccharides are degraded or converted to xylose, mannose, galactose, arabinose or a combination thereof at a higher rate, as compared to non-transformed plants, the plant cell wall acetate concentration is decreased, as compared to non-transformed plants, the plant cell wall feruloylation is decreased, as compared to non-transformed plants, the plant cell wall cross-linking is decreased, as compared to non-transformed plants, and/or the plant is selected from the group consisting of: maize, soybean, sunflower, sorghum, canola, wheat, alfalfa, cotton, rice, barley, millet, peanut, sugar cane, grass, turfgrass miscanthus, switchgrass and cocoa.

Also contemplated is a method of modulating plant tissue growth with a Golgi targeted enzyme in a plant, comprising expressing a recombinant expression cassette comprising the polynucleotide of the previous methods operably linked to a promoter. In addition to this the method wherein: the plant is selected from the group consisting of: maize, soybean, sorghum, canola, wheat, alfalfa, cotton, rice, barley, millet, peanut, sugar cane, grass, turfgrass, miscanthus, switchgrass and cocoa, the plant has improved silage quality and digestibility, the promoter is selected from the group consisting of a leaf specific promoter, vascular element preferred promoter and a root specific promoter.

An embodiment of the disclosure includes the methods previously mentioned comprising expressing a polynucleotide that encodes a polypeptide having at least 85% sequence similarity to a polypeptide selected from the group consisting of SEQ ID NOS: 4-18, 59, 62, 65, 68, 70 and 71.

One embodiment would be a transgenic plant cell of the previous methods, with altered cell wall content comprising a recombinant expression cassette comprising expressing a polynucleotide that encodes a polypeptide having at least 85% sequence similarity to a polypeptide selected from the group consisting of SEQ ID NOS: 4-18, 59, 62, 65, 68, 70 and 71, wherein the plant is: a monocot, a dicot, selected from the group consisting of: maize, soybean, sunflower, sorghum, canola, grass, sugarcane, wheat, alfalfa, cotton, rice, barley, miscanthus, turfgrass, switchgrass and millet.

Also an embodiment is a method of modulating plant carbohydrate concentration in a transgenic plant, the method comprising expressing a recombinant polynucleotide encoding the Golgi targeting enzyme of one of the aforementioned methods.

In addition, the method of altering the cross-linking and acetyl content in plant tissues in order to improve the quality of biomass available for biofuels in a plant, the method comprising the steps of: transforming a plant cell with a recombinant expression cassette comprising a polynucleotide having at least 85% sequence identity to the full length sequence of a enzyme encoding polynucleotide selected from the group consisting of SEQ ID NO: 4-18, 59, 62, 65, 68, 70 and 71, operably linked to a promoter; culturing the plant cell under plant-forming conditions to express the polypeptide enzyme in the plant tissue; growing the transformed plant tissue under plant tissue growing conditions; wherein the composition of the Golgi polysaccharides in said transformed plant cell is altered and processing the transformed plant tissue to obtain biofuel.

Also contemplated is a method of producing biomass for silage or biofuel production comprising providing plant tissue having a substantially lowered amount of acetate or ferulate content, wherein the plant tissue expresses a recombinant esterase that is targeted to a compartment within the Golgi apparatus. Another embodiment is this same method, wherein the polypeptide comprises at least 85% sequence similarity to a polypeptide selected from the group consisting of SEQ ID NOS: 4-18, 59, 62, 65, 68, 70 and 71.

An additional embodiment would be a product derived from the method of processing of transgenic plant component expressing an isolated polynucleotide encoding a Golgi targeting enzyme, the method comprising the steps: growing a plant that expresses a polynucleotide having at least 85% sequence identity to the full length sequence of SEQ ID NO: 4-18, 59, 62, 65, 68, 70 and 71, operably linked to a promoter, and processing the plant component to obtain a product, and the product which is a constituent of ethanol.

Another embodiment is a plant stover comprising a reduced acetyl or feruloyl content due to the targeting of a recombinant esterase to the Golgi apparatus, wherein the esterase catalyzes the cleavage of the acetyl or feruloyl molecules which includes: corn stover, stover used for the production of biofuel comprising butanol and/or ethanol.

An additional embodiment would be a method of reducing the overall acetate and/or ferulate content in a plant tissue, the method comprising expressing an inhibitory nucleotide molecule that suppresses the expression of an acetyl or a feruloyl transferase.

### BRIEF DESCRIPTION OF THE DRAWINGS

FIG. 1: Hemicellulose polysaccharide in maize stover (Glucuronoarabinoxylan) structure (Dhugga, 2007).

FIG. 2: Arabidopsis alpha-1,2-xylosyltransferase directed GFP expression in transgenic plants.

FIG. 3: Effect of NaOH concentration and time of incubation on acetate release/extractability in maize stover.

FIG. 4: Determination of absorbance at A₃₄₀ using 96-channel and 8-channel pipetors for the quantification of acetate.

FIG. 5: Cell wall acetate in Arabidopsis transgenic (T₁) expressing a bacterial or a fungal esterase under the control of 35S promoter.

FIG. 6: Stalk acetate content in FastCorn T₁ events expressing acetyl esterase with S2A promoter.

FIG. 7: Xylose/arabinose ratio in Arabidopsis transgenics expressing fungal/bacterial arabinosidase under the control of 35S promoter.

FIG. 8: Wall ferulate content in T₀ maize events expressing Golgi-targeted feruolyl esterase under the control of S2A promoter.

FIG. 9: Variation of cell wall acetate content in genetic diversity set for mature cob tissue.

FIG. 10: Association genetics of cob acetate content identified a strong QTL at chromosome 3.

FIG. 11: Cell wall acetate content in a T-DNA mutant of putative pectin acetylesterase in Arabidopsis. Inset shows the map location of T-DNA insertion.

FIG. 12: Reduction in wall acetate in T₀ plants overexpressing Arabidopsis pectin acetylesterase (AT3G09410) under the control of 35S and S2A promoters.

FIG. 13: (13A-13C) Alignment of related Glucuronosyltransgerase genes from Maize and Arabidopsis. The identical residues are in bold text and underlined, with similar residues being marked with bold italics (50% identity), or italics (75% identity).

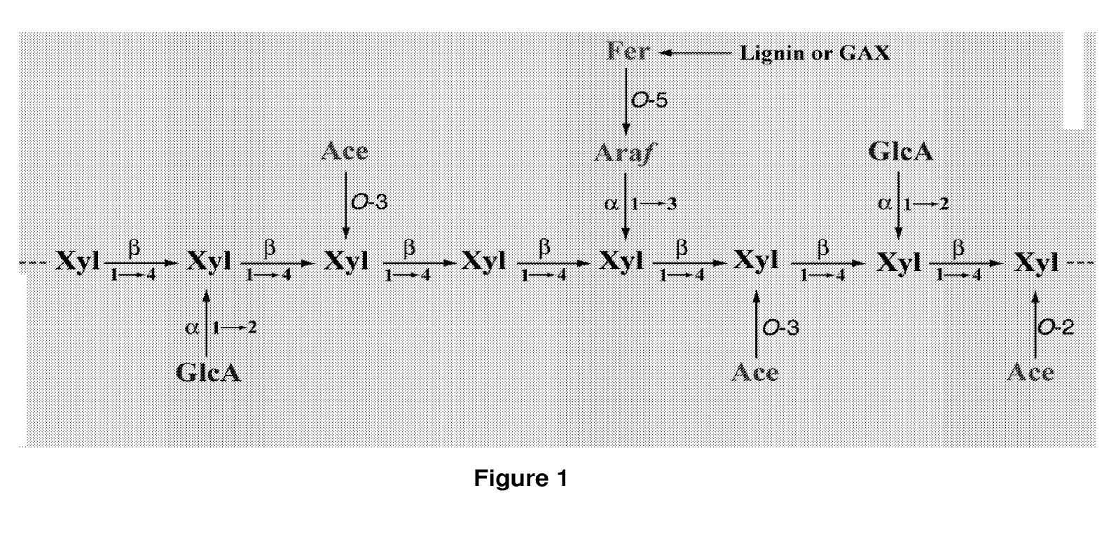

### DETAILED DESCRIPTION

### Overview

### A. Nucleic Acids and Protein

Unless otherwise stated, the polynucleotide and polypeptide sequences, subsequences thereof and functional domains thereof identified in Table 1 represent polynucleotides and polypeptides of the present disclosure. Table 1 cross-references these polynucleotide and polypeptides to their gene name and internal database identification number (SEQ ID NO.). A nucleic acid of the present disclosure comprises a polynucleotide of the present disclosure. A protein of the present disclosure comprises a polypeptide of the present disclosure.

TABLE 1

| | PN/PP | | |
|---|---|---|---|
| | Polynucleotide/ | | |
| SEQ ID NOS: | polypeptide | ORGANISM | NAME |
| SEQ ID NO: 1 | PP | Arabidopsis | alpha mannose II |
| | | thaliana | |
| SEQ ID NO: 2 | PP | Arabidopsis | Alpha-1,2 xylosyltransferase |
| | | thaliana | |
| SEQ ID NO: 3 | PP | Rat | Alpha-2,6-sialyltransferase |
| SEQ ID NO: 4 | PP | Aspergillus ficuum | Acetyl xylan esterase |
| SEQ ID NO: 5 | PP | Aspergillus niger | Acetyl xylan esterase |
| SEQ ID NO: 6 | PP | Aspergillus oryzae | Acetyl xylan esterase |
| SEQ ID NO: 7 | PP | Aspergillus | Acetyl xylan esterase |
| | | clavatus | |
| SEQ ID NO: 8 | PP | Clostridium | Acetyl xylan esterase |
| | | thermocellum | |
| SEQ ID NO: 9 | PP | Neurospora crassa | Acetyl xylan esterase |
| SEQ ID NO: 10 | PP | Penicillium | Feruloyl esterase |
| | | funiculosum | |
| SEQ ID NO: 11 | PP | Aspergillus niger | Feruloyl esterase |
| SEQ ID NO: 12 | PP | Aspergillus niger | Feruloyl esterase |
| SEQ ID NO: 13 | PP | Clostridium | Feruloyl esterase |
| | | thermocellum | |
| SEQ ID NO: 14 | PP | Neurospora crassa | Feruloyl esterase |
| SEQ ID NO: 15 | PP | Clostridium | arabinosidase |
| | | thermocellum | |
| SEQ ID NO: 16 | PP | Bacillus subtillis | arabinosidase |
| SEQ ID NO: 17 | PP | Aspergillus oryzae | arabinosidase |
| SEQ ID NO: 18 | PP | Aspergillus niger | arabinosidase |
| SEQ ID NO: 19 | PN | Arabidopsis | mannose II primer |
| | | thaliana | |
| SEQ ID NO: 20 | PN | Arabidopsis | xylosyltransferase primer |
| | | thaliana | |
| SEQ ID NO: 21 | PN | Arabidopsis | mannose II primer |
| | | thaliana | |
| SEQ ID NO: 22 | PN | Arabidopsis | xylosyltransferase primer |
| | | thaliana | |
| SEQ ID NO: 23 | PN | Aspergillus niger | Acetyl xylan esterase primer |
| SEQ ID NO: 24 | PN | Aspergillus niger | Acetyl xylan esterase primer |
| SEQ ID NO: 25 | PN | Aspergillus oryzae | Acetyl xylan esterase primer |
| SEQ ID NO: 26 | PN | Aspergillus oryzae | Acetyl xylan esterase primer |
| SEQ ID NO: 27 | PN | Aspergillus | Acetyl xylan esterase primer |
| | | clavatus | |
| SEQ ID NO: 28 | PN | Aspergillus | Acetyl xylan esterase primer |
| | | clavatus | |
| SEQ ID NO: 29 | PN | Clostridium | Acetyl xylan esterase primer |
| | | thermocellum | |
| SEQ ID NO: 30 | PN | Clostridium | Acetyl xylan esterase primer |
| | | thermocellum | |
| SEQ ID NO: 31 | PN | Neurospora crassa | Acetyl xylan esterase primer |
| SEQ ID NO: 32 | PN | Neurospora crassa | Acetyl xylan esterase primer |
| SEQ ID NO: 33 | PN | Aspergillus niger | Feruloyl esterase primer |
| SEQ ID NO: 34 | PN | Aspergillus niger | Feruloyl esterase primer |
| SEQ ID NO: 35 | PN | Aspergillus niger | Feruloyl esterase primer |
| SEQ ID NO: 36 | PN | Aspergillus niger | Feruloyl esterase primer |
| SEQ ID NO: 37 | PN | Clostridium | Feruloyl esterase primer |
| | | thermocellum | |
| SEQ ID NO: 38 | PN | Clostridium | Feruloyl esterase primer |
| | | thermocellum | |
| SEQ ID NO: 39 | PN | Neurospora crassa | Feruloyl esterase primer |
| SEQ ID NO: 40 | PN | Neurospora crassa | Feruloyl esterase primer |
| SEQ ID NO: 41 | PN | Penicillium | Feruloyl esterase primer |
| | | funiculosum | |
| SEQ ID NO: 42 | PN | Penicillium | Feruloyl esterase primer |
| | | funiculosum | |
| SEQ ID NO: 43 | PN | Aspergillus niger | arabinosidase primer |
| SEQ ID NO: 44 | PN | Aspergillus niger | arabinosidase primer |
| SEQ ID NO: 45 | PN | Aspergillus oryzae | arabinosidase primer |
| SEQ ID NO: 46 | PN | Aspergillus oryzae | arabinosidase primer |
| SEQ ID NO: 47 | PN | Bacillus subtilis | arabinosidase primer |
| SEQ ID NO: 48 | PN | Bacillus subtilis | arabinosidase primer |
| SEQ ID NO: 49 | PN | Clostridium | arabinosidase primer |
| | | thermocellum | |
| SEQ ID NO: 50 | PN | Clostridium | arabinosidase primer |
| | | thermocellum | |
| SEQ ID NO: 51 | PN | Clostridium | arabinosidase primer |
| | | thermocellum | |
| SEQ ID NO: 52 | PN | Artificial sequence | 5′ bar primer |
| SEQ ID NO: 53 | PN | Artificial sequence | 3′ bar primer |
| SEQ ID NO: 54 | PN | Zea maize | pco593184 transcript |
| SEQ ID NO: 55 | PN | Zea maize | ORF |
| SEQ ID NO: 59 | PP | Zea maize | Polypeptide |
| SEQ ID NO: 57 | PN | Zea maize | Transcript |
| SEQ ID NO: 58 | PN | Zea maize | ORF |
| SEQ ID NO: 59 | PP | Zea maize | Polypeptide |
| SEQ ID NO: 60 | PN | Zea maize | Transcript |
| SEQ ID NO: 61 | PN | Zea maize | ORF |
| SEQ ID NO: 62 | PP | Zea maize | Polypeptide |
| SEQ ID NO: 63 | PN | Zea maize | Transcript |
| SEQ ID NO: 64 | PN | Zea maize | ORF |
| SEQ ID NO: 65 | PP | Zea maize | Polypeptide |
| SEQ ID NO: 66 | PN | Zea maize | Transcript |
| SEQ ID NO: 67 | PN | Zea maize | ORF |
| SEQ ID NO: 68 | PP | Zea maize | Polypeptide |
| SEQ ID NO: 69 | PN | consensus | polypeptide |
| SEQ ID NO: 70 | PP | Arabidopsis | Polypeptide |
| | | thaliana | |
| SEQ ID NO: 71 | PP | Aragidopsis | polypeptide |
| | | thaliana | |

The following table (Table 2) contains a repertory of constructs made from three different organisms per enzyme, four targeting sequences and two promoters

TABLE 2

| | Enzyme/ | ManII | | XylT | | SialT | | None | |
|---|---|---|---|---|---|---|---|---|---|
| Organism | Protein | 35S | S2A | 35S | S2A | 35S | S2A | 35S | S2A |
| Aspergillus oryzae | Acetyl esterase | + | + | + | + | + | + | + | + |
| Neurospora crassa | Acetyl esterase | + | + | + | + | + | + | + | + |
| Clostridium | Acetyl esterase | + | + | + | + | + | + | + | + |
| thermocellum | | | | | | | | | |
| Aspergillus niger | Feruloyl esterase | + | + | + | + | + | + | + | + |
| Neurospora crassa | Feruloyl esterase | + | + | + | + | + | + | + | + |
| Clostridium | Feruloyl esterase | + | + | + | + | + | + | + | + |
| thermocellum | | | | | | | | | |
| Aspergillus niger | Arabinosidase | + | + | + | + | + | + | + | + |
| Bacillus subtilis | Arabinosidase | + | + | + | + | + | + | + | + |
| Clostridium | Arabinosidase | + | + | + | + | + | + | + | + |
| thermocellum | | | | | | | | | |
| Jellyfish | GFP | + | − | + | − | + | − | − | − |

### B. Exemplary Utility of the Present Disclosure

This disclosure demonstrates that one can obtain stable transgenic lines in Arabidopsis and maize with a consistently lower level of acetate or ferulate by targeting respective esterases to the Golgi apparatus using three different targeting signals (Saint-Jore-Dupas, et al., (2004) Cellular and Molecular Life Sciences 61:159-171). Any reduction in acetate content of the cell wall and its substitution by polysaccharides would improve the efficiency of biofuels production from the crop residue. This disclosure reports a consistent reduction in wall acetate content.

The present disclosure provides utility in such exemplary applications as direct down regulation of the degree of feruloylation and cross-linking as well as acetyl content in the plants, which leads to improved quality of biomass for biofuels and silage digestability. In addition interference with the biosynthesis of Golgi polysaccharides by expressing glycosidases or esterases is expected to altered cell wall composition in the plants, leading to improvement in the biomass quality for biofuel production. Improvement of stalk quality for improved standability or silage digestibility also might result from this approach.

The disclosure describes reducing the plant cell wall acetate content by targeting bacterial or fungal acetyl or feruloyl esterases to the Golgi apparatus. The target reduction of acetate by any or a combination of these esterases will at least be about 1%, 5%, 10%, 15%, 20%, 20%, 25%, 30%, 35%, 40%, 45%, 50%, 55%, 60%, 65%, 70%, 75%, 80%, 85% to about 90% or greater. Preferred range of acetate reduction is 30-50%.

The disclosure describes reducing the plant cell wall acetate content by selectively targeting bacterial, fungal or plant acetyl or feruloyl esterases to the Golgi apparatus. In an embodiment, these esterases are selectively targeted to the Golgi, such that the activity of these esterases in the Golgi is at least about 25%, 30%, 35%, 40%, 45%, 50%, 55%, 60%, 65%, 70%, 75%, 80%, 85% to about 90% or greater as compared to the total activity. In a preferred embodiment, the esterases have substantial activity in the Golgi as compared to the activity in other non-Golgi cellular components.

### DEFINITIONS

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole. Section headings provided throughout the specification are not limitations to the various objects and embodiments of the present disclosure.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology: Principles and Applications, Persing, et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal and fungal mitochondria, the bacterium Mycoplasma capricolum or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledons or dicotyledons as these preferences have been shown to differ (Murray, et al., (1989) Nucl. Acids Res. 17:477-498). Thus, the maize preferred codon for a particular amino acid may be derived from known gene sequences from maize. Maize codon usage for 28 genes from maize plants is listed in Table 4 of Murray, et al., supra.

As used herein “full-length sequence” in reference to a specified polynucleotide or its encoded protein means having the entire amino acid sequence of a native (non-synthetic), endogenous, biologically (e.g., structurally or catalytically) active form of the specified protein. Methods to determine whether a sequence is full-length are well known in the art, including such exemplary techniques as northern or western blots, primer extension, S1 protection and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present disclosure. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign species, or, if from the same species, is substantially modified from its native form in composition and/or genomic locus by human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by human intervention.

By “host cell” is meant a cell which contains a vector and supports the replication and/or expression of the vector. Host cells may be prokaryotic cells such as E. coli, or eukaryotic cells such as yeast, insect, amphibian or mammalian cells. Preferably, host cells are monocotyledonous or dicotyledonous plant cells. A particularly preferred monocotyledonous host cell is a maize host cell.

The term “introduced” includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection”, “transformation” and “transduction”.

The term “isolated” refers to material, such as a nucleic acid or a protein, which is: (1) substantially or essentially free from components which normally accompany or interact with it as found in its natural environment. The isolated material optionally comprises material not found with the material in its natural environment or (2) if the material is in its natural environment, the material has been synthetically altered or synthetically produced by deliberate human intervention and/or placed at a different location within the cell. The synthetic alteration or creation of the material can be performed on the material within or apart from its natural state. For example, a naturally-occurring nucleic acid becomes an isolated nucleic acid if it is altered or produced by non-natural, synthetic methods or if it is transcribed from DNA which has been altered or produced by non-natural, synthetic methods. The isolated nucleic acid may also be produced by the synthetic re-arrangement (“shuffling”) of a part or parts of one or more allelic forms of the gene of interest. Likewise, a naturally-occurring nucleic acid (e.g., a promoter) becomes isolated if it is introduced to a different locus of the genome. Nucleic acids which are “isolated,” as defined herein, are also referred to as “heterologous” nucleic acids. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells, Zarling, et al., WO 1993/22443 (PCT/US93/03868).

As used herein, “nucleic acid” includes reference to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism, tissue or of a cell type from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook, et al., Molecular Cloning—A Laboratory Manual, 2^{nd} ed., Vol. 1-3 (1989) and Current Protocols in Molecular Biology, Ausubel, et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen and microspores. Plant cells can also be understood to include modified cells, such as protoplasts, obtained from the aforementioned tissues. The class of plants which can be used in the methods of the disclosure is generally as broad as the class of higher plants amenable to transformation techniques, including both monocotyledonous and dicotyledonous plants. A particularly preferred plant is Zea mays.

As used herein, “polynucleotide” includes reference to a deoxyribopolynucleotide, ribopolynucleotide or chimeras or analogs thereof that have the essential nature of a natural deoxy- or ribo-nucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. Further, this disclosure contemplates the use of both the methionine-containing and the methionine-less amino terminal variants of the protein of the disclosure.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses and bacteria which comprise genes expressed in plant cells such Agrobacterium or Rhizobium. Examples of promoters under developmental control include promoters that preferentially initiate transcription in certain tissues, such as leaves, roots or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions or the presence of light. Tissue specific, tissue preferred, cell type specific and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of human intervention. The term “recombinant” as used herein does not encompass the alteration of the cell or vector by naturally occurring events (e.g., spontaneous mutation, natural transformation/transduction/transposition) such as those occurring without human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity and most preferably 100% sequence identity (i.e., complementary) with each other.

The term “stringent conditions” or “stringent hybridization conditions” includes reference to conditions under which a probe will selectively hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (“Tₘ”) for the specific sequence and its complement at a defined ionic strength and pH. However, severely stringent conditions can utilize a hybridization and/or wash at 1, 2, 3 or 4° C. lower than the Tₘ; moderately stringent conditions can utilize a hybridization and/or wash at 6, 7, 8, 9 or 10° C. lower than the Tₘ; low stringency conditions can utilize a hybridization and/or wash at 11, 12, 13, 14, 15 or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. Hybridization and/or wash conditions can be applied for at least 10, 30, 60, 90, 120 or 240 minutes. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993) and Current Protocols in Molecular Biology, Chapter 2, Ausubel, et al., Eds., Greene Publishing and Wiley-Interscience, New York (1995).

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in introduction of a polynucleotide of the present disclosure into a host cell. Vectors are often replicons. Expression vectors permit transcription of a nucleic acid inserted therein.

The following terms are used to describe the sequence relationships between a polynucleotide/polypeptide of the present disclosure with a reference polynucleotide/polypeptide: (a) “reference sequence”, (b) “comparison window”, (c) “sequence identity” and (d) “percentage of sequence identity”.

(a) As used herein, “reference sequence” is a defined sequence used as a basis for sequence comparison with a polynucleotide/polypeptide of the present disclosure. A reference sequence may be a subset or the entirety of a specified sequence; for example, as a segment of a full-length cDNA or gene sequence or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100 or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, (1981) Adv. Appl. Math. 2:482; by the homology alignment algorithm of Needleman and Wunsch, (1970) J. Mol. Biol. 48:443; by the search for similarity method of Pearson and Lipman, (1988) Proc. Natl. Acad. Sci. 85:2444; by computerized implementations of these algorithms, including, but not limited to: CLUSTAL in the PC/Gene program by Intelligenetics, Mountain View, Calif.; GAP, BESTFIT, BLAST, FASTA and TFASTA in the Wisconsin Genetics Software Package®, Genetics Computer Group (GCG®), 575 Science Dr., Madison, Wis., USA; the CLUSTAL program is well described by Higgins and Sharp, (1988) Gene 73:237-244; Higgins and Sharp, (1989) CABIOS 5:151-153; Corpet, et al., (1988) Nucleic Acids Research 16:10881-90; Huang, et al., (1992) Computer Applications in the Biosciences 8:155-65 and Pearson, et al., (1994) Methods in Molecular Biology 24:307-331.

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul, et al., (1990) J. Mol. Biol., 215:403-410 and Altschul, et al., (1997) Nucleic Acids Res. 25:3389-3402.

Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff and Henikoff, (1989) Proc. Natl. Acad. Sci. USA 89:10915).

In addition to calculating percent sequence identity, the BLAST algorithm also performs a statistical analysis of the similarity between two sequences (see, e.g., Karlin and Altschul, (1993) Proc. Nat'l. Acad. Sci. USA 90:5873-5877). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-163) and XNU (Claverie and States, (1993) Comput. Chem 17:191-201) low-complexity filters can be employed alone or in combination.

Unless otherwise stated, nucleotide and protein identity/similarity values provided herein are calculated using GAP (GCG® Version 10) under default values.

GAP (Global Alignment Program) can also be used to compare a polynucleotide or polypeptide of the present disclosure with a reference sequence. GAP uses the algorithm of Needleman and Wunsch, (J. Mol. Biol. 48: 443-453 (1970)) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 0, 1, 2, 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Multiple alignment of the sequences can be performed using the CLUSTAL method of alignment (Higgins and Sharp, (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17 e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

### Utilities

The present disclosure provides, among other things, compositions and methods for modulating (i.e., increasing or decreasing) the level of polynucleotides and polypeptides of the present disclosure in plants. In particular, the polynucleotides and polypeptides of the present disclosure can be expressed temporally or spatially, e.g., at developmental stages, in tissues and/or in quantities, which are uncharacteristic of non-recombinantly engineered plants.

The present disclosure also provides isolated nucleic acids comprising polynucleotides of sufficient length and complementarity to a polynucleotide of the present disclosure to use as probes or amplification primers in the detection, quantitation or isolation of gene transcripts. For example, isolated nucleic acids of the present disclosure can be used as probes in detecting deficiencies in the level of mRNA in screenings for desired transgenic plants, for detecting mutations in the gene (e.g., substitutions, deletions or additions), for monitoring upregulation of expression or changes in enzyme activity in screening assays of compounds, for detection of any number of allelic variants (polymorphisms), orthologs or paralogs of the gene or for site directed mutagenesis in eukaryotic cells (see, e.g., U.S. Pat. No. 5,565,350). The isolated nucleic acids of the present disclosure can also be used for recombinant expression of their encoded polypeptides or for use as immunogens in the preparation and/or screening of antibodies. The isolated nucleic acids of the present disclosure can also be employed for use in sense or antisense suppression of one or more genes of the present disclosure in a host cell, tissue or plant. Attachment of chemical agents which bind, intercalate, cleave and/or crosslink to the isolated nucleic acids of the present disclosure can also be used to modulate transcription or translation.

The present disclosure also provides isolated proteins comprising a polypeptide of the present disclosure (e.g., preproenzyme, proenzyme or enzymes). The present disclosure also provides proteins comprising at least one epitope from a polypeptide of the present disclosure. The proteins of the present disclosure can be employed in assays for enzyme agonists or antagonists of enzyme function or for use as immunogens or antigens to obtain antibodies specifically immunoreactive with a protein of the present disclosure. Such antibodies can be used in assays for expression levels, for identifying and/or isolating nucleic acids of the present disclosure from expression libraries, for identification of homologous polypeptides from other species or for purification of polypeptides of the present disclosure.

The isolated nucleic acids and polypeptides of the present disclosure can be used over a broad range of plant types, particularly monocots such as the species of the family Gramineae including Hordeum, Secale, Oryza, Triticum, Sorghum (e.g., S. bicolor) and Zea (e.g., Z. mays) and dicots such as Glycine.

The isolated nucleic acid and proteins of the present disclosure can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browallia, Pisum, Phaseolus, Lolium and Avena.

### Nucleic Acids

The present disclosure provides, among other things, isolated nucleic acids of RNA, DNA and analogs and/or chimeras thereof, comprising a polynucleotide of the present disclosure.

A polynucleotide of the present disclosure is inclusive of those in Table 1 and:

(a) an isolated polynucleotide encoding a polypeptide of the present disclosure such as those referenced in Table 1, including exemplary polynucleotides of the present disclosure;

(b) an isolated polynucleotide which is the product of amplification from a plant nucleic acid library using primer pairs which selectively hybridize under stringent conditions to loci within a polynucleotide of the present disclosure;

(c) an isolated polynucleotide which selectively hybridizes to a polynucleotide of (a) or (b);

(d) an isolated polynucleotide having a specified sequence identity with polynucleotides of (a), (b) or (c);

(e) an isolated polynucleotide encoding a protein having a specified number of contiguous amino acids from a prototype polypeptide, wherein the protein is specifically recognized by antisera elicited by presentation of the protein and wherein the protein does not detectably immunoreact to antisera which has been fully immunosorbed with the protein;

(f) complementary sequences of polynucleotides of (a), (b), (c), (d) or (e);

(g) an isolated polynucleotide comprising at least a specific number of contiguous nucleotides from a polynucleotide of (a), (b), (c), (d), (e) or (f);

(h) an isolated polynucleotide from a full-length enriched cDNA library having the physico-chemical property of selectively hybridizing to a polynucleotide of (a), (b), (c), (d), (e), (f) or (g);

(i) an isolated polynucleotide made by the process of: 1) providing a full-length enriched nucleic acid library, 2) selectively hybridizing the polynucleotide to a polynucleotide of (a), (b), (c), (d), (e), (f), (g) or (h), thereby isolating the polynucleotide from the nucleic acid library.

### A. Polynucleotides Encoding a Polypeptide of the Present Disclosure

As indicated in (a), above, the present disclosure provides isolated nucleic acids comprising a polynucleotide of the present disclosure, wherein the polynucleotide encodes a polypeptide of the present disclosure. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine and UGG, which is ordinarily the only codon for tryptophan) can be modified to yield a functionally identical molecule. Thus, each silent variation of a nucleic acid which encodes a polypeptide of the present disclosure is implicit in each described polypeptide sequence and is within the scope of the present disclosure. Accordingly, the present disclosure includes polynucleotides of the present disclosure and polynucleotides encoding a polypeptide of the present disclosure.

B. Polynucleotides Amplified from a Plant Nucleic Acid Library

As indicated in (b), above, the present disclosure provides an isolated nucleic acid comprising a polynucleotide of the present disclosure, wherein the polynucleotides are amplified, under nucleic acid amplification conditions, from a plant nucleic acid library. Nucleic acid amplification conditions for each of the variety of amplification methods are well known to those of ordinary skill in the art. The plant nucleic acid library can be constructed from a monocot such as a cereal crop. Exemplary cereals include maize, sorghum, alfalfa, canola, wheat or rice. The plant nucleic acid library can also be constructed from a dicot such as soybean. Zea mays lines B73, PHRE1, A632, BMS-P2#10, W23 and Mo17 are known and publicly available. Other publicly known and available maize lines can be obtained from the Maize Genetics Cooperation (Urbana, Ill.). Wheat lines are available from the Wheat Genetics Resource Center (Manhattan, Kans.).

The nucleic acid library may be a cDNA library, a genomic library or a library generally constructed from nuclear transcripts at any stage of intron processing. cDNA libraries can be normalized to increase the representation of relatively rare cDNAs. In optional embodiments, the cDNA library is constructed using an enriched full-length cDNA synthesis method. Examples of such methods include Oligo-Capping (Maruyama and Sugano, (1994) Gene 138:171-174), Biotinylated CAP Trapper (Carninci, et al., (1996) Genomics 37:327-336) and CAP Retention Procedure (Edery, et al., (1995) Molecular and Cellular Biology 15:3363-3371). Rapidly growing tissues or rapidly dividing cells are preferred for use as an mRNA source for construction of a cDNA library. Growth stages of maize are described in “How a Corn Plant Develops,” Special Report Number 48, Iowa State University of Science and Technology Cooperative Extension Service, Ames, Iowa, Reprinted February 1993.

A polynucleotide of this embodiment (or subsequences thereof) can be obtained, for example, by using amplification primers which are selectively hybridized and primer extended, under nucleic acid amplification conditions, to at least two sites within a polynucleotide of the present disclosure, or to two sites within the nucleic acid which flank and comprise a polynucleotide of the present disclosure, or to a site within a polynucleotide of the present disclosure and a site within the nucleic acid which comprises it. Methods for obtaining 5′ and/or 3′ ends of a vector insert are well known in the art. See, e.g., RACE (Rapid Amplification of Complementary Ends) as described in Frohman, in PCR Protocols: A Guide to Methods and Applications, Innis, et al., Eds. (Academic Press, Inc., San Diego), pp. 28-38 (1990)); see, also, U.S. Pat. No. 5,470,722 and Current Protocols in Molecular Biology, Unit 15.6, Ausubel, et al., Eds., Greene Publishing and Wiley-Interscience, New York (1995); Frohman and Martin, Techniques 1:165 (1989).

Optionally, the primers are complementary to a subsequence of the target nucleic acid which they amplify but may have a sequence identity ranging from about 85% to 99% relative to the polynucleotide sequence which they are designed to anneal to. As those skilled in the art will appreciate, the sites to which the primer pairs will selectively hybridize are chosen such that a single contiguous nucleic acid can be formed under the desired nucleic acid amplification conditions. The primer length in nucleotides is selected from the group of integers consisting of from at least 15 to 50. Thus, the primers can be at least 15, 18, 20, 25, 30, 40 or 50 nucleotides in length. Those of skill will recognize that a lengthened primer sequence can be employed to increase specificity of binding (i.e., annealing) to a target sequence. A non-annealing sequence at the 5′end of a primer (a “tail”) can be added, for example, to introduce a cloning site at the terminal ends of the amplicon.

The amplification products can be translated using expression systems well known to those of skill in the art. The resulting translation products can be confirmed as polypeptides of the present disclosure by, for example, assaying for the appropriate catalytic activity (e.g., specific activity and/or substrate specificity) or verifying the presence of one or more epitopes which are specific to a polypeptide of the present disclosure. Methods for protein synthesis from PCR derived templates are known in the art and available commercially. See, e.g., Amersham Life Sciences, Inc, Catalog '97, p. 354.

C. Polynucleotides which Selectively Hybridize to a Polynucleotide of (A) or (B)

As indicated in (c), above, the present disclosure provides isolated nucleic acids comprising polynucleotides of the present disclosure, wherein the polynucleotides selectively hybridize, under selective hybridization conditions, to a polynucleotide of sections (A) or (B) as discussed above. Thus, the polynucleotides of this embodiment can be used for isolating, detecting, and/or quantifying nucleic acids comprising the polynucleotides of (A) or (B). For example, polynucleotides of the present disclosure can be used to identify, isolate, or amplify partial or full-length clones in a deposited library. In some embodiments, the polynucleotides are genomic or cDNA sequences isolated or otherwise complementary to a cDNA from a dicot or monocot nucleic acid library. Exemplary species of monocots and dicots include, but are not limited to: maize, canola, soybean, cotton, wheat, sorghum, sunflower, alfalfa, oats, sugar cane, millet, barley and rice. The cDNA library comprises at least 50% to 95% full-length sequences (for example, at least 50%, 60%, 70%, 80%, 90% or 95% full-length sequences). The cDNA libraries can be normalized to increase the representation of rare sequences. See, e.g., U.S. Pat. No. 5,482,845. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% to 80% sequence identity and can be employed to identify orthologous or paralogous sequences.

D. Polynucleotides Having a Specific Sequence Identity with the Polynucleotides of (A), (B) or (C)

As indicated in (d), above, the present disclosure provides isolated nucleic acids comprising polynucleotides of the present disclosure, wherein the polynucleotides have a specified identity at the nucleotide level to a polynucleotide as disclosed above in sections (A), (B) or (C), above. Identity can be calculated using, for example, the BLAST, CLUSTALW or GAP algorithms under default conditions. The percentage of identity to a reference sequence is at least 50% and, rounded upwards to the nearest integer, can be expressed as an integer selected from the group of integers consisting of from 50 to 99. Thus, for example, the percentage of identity to a reference sequence can be at least 60%, 70%, 75%, 80%, 85%, 90% or 95%.

Optionally, the polynucleotides of this embodiment will encode a polypeptide that will share an epitope with a polypeptide encoded by the polynucleotides of sections (A), (B) or (C). Thus, these polynucleotides encode a first polypeptide which elicits production of antisera comprising antibodies which are specifically reactive to a second polypeptide encoded by a polynucleotide of (A), (B) or (C). However, the first polypeptide does not bind to antisera raised against itself when the antisera has been fully immunosorbed with the first polypeptide. Hence, the polynucleotides of this embodiment can be used to generate antibodies for use in, for example, the screening of expression libraries for nucleic acids comprising polynucleotides of (A), (B) or (C), or for purification of, or in immunoassays for, polypeptides encoded by the polynucleotides of (A), (B) or (C). The polynucleotides of this embodiment comprise nucleic acid sequences which can be employed for selective hybridization to a polynucleotide encoding a polypeptide of the present disclosure.

Screening polypeptides for specific binding to antisera can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Numbers 1991/17271, 1991/18980, 1991/19818 and 1993/08278. Other systems for generating libraries of peptides have aspects of both in vitro chemical synthesis and recombinant methods. See, PCT Patent Publication Numbers 1992/05258, 1992/14843 and 1997/20078. See also, U.S. Pat. Nos. 5,658,754 and 5,643,768. Peptide display libraries, vectors, and screening kits are commercially available from such suppliers as Invitrogen (Carlsbad, Calif.).

E. Polynucleotides Encoding a Protein Having a Subsequence from a Prototype Polypeptide and Cross-Reactive to the Prototype Polypeptide

As indicated in (e), above, the present disclosure provides isolated nucleic acids comprising polynucleotides of the present disclosure, wherein the polynucleotides encode a protein having a subsequence of contiguous amino acids from a prototype polypeptide of the present disclosure such as are provided in (a), above. The length of contiguous amino acids from the prototype polypeptide is selected from the group of integers consisting of from at least 10 to the number of amino acids within the prototype sequence. Thus, for example, the polynucleotide can encode a polypeptide having a subsequence having at least 10, 15, 20, 25, 30, 35, 40, 45 or 50, contiguous amino acids from the prototype polypeptide. Further, the number of such subsequences encoded by a polynucleotide of the instant embodiment can be any integer selected from the group consisting of from 1 to 20, such as 2, 3, 4 or 5. The subsequences can be separated by any integer of nucleotides from 1 to the number of nucleotides in the sequence such as at least 5, 10, 15, 25, 50, 100 or 200 nucleotides.

The proteins encoded by polynucleotides of this embodiment, when presented as an immunogen, elicit the production of polyclonal antibodies which specifically bind to a prototype polypeptide such as but not limited to, a polypeptide encoded by the polynucleotide of (a) or (b), above. Generally, however, a protein encoded by a polynucleotide of this embodiment does not bind to antisera raised against the prototype polypeptide when the antisera has been fully immunosorbed with the prototype polypeptide. Methods of making and assaying for antibody binding specificity/affinity are well known in the art. Exemplary immunoassay formats include ELISA, competitive immunoassays, radioimmunoassays, Western blots, indirect immunofluorescent assays and the like.

In a preferred assay method, fully immunosorbed and pooled antisera which is elicited to the prototype polypeptide can be used in a competitive binding assay to test the protein. The concentration of the prototype polypeptide required to inhibit 50% of the binding of the antisera to the prototype polypeptide is determined. If the amount of the protein required to inhibit binding is less than twice the amount of the prototype protein, then the protein is said to specifically bind to the antisera elicited to the immunogen. Accordingly, the proteins of the present disclosure embrace allelic variants, conservatively modified variants and minor recombinant modifications to a prototype polypeptide.

A polynucleotide of the present disclosure optionally encodes a protein having a molecular weight as the non-glycosylated protein within 20% of the molecular weight of the full-length non-glycosylated polypeptides of the present disclosure. Molecular weight can be readily determined by SDS-PAGE under reducing conditions. Optionally, the molecular weight is within 15% of a full length polypeptide of the present disclosure, more preferably within 10% or 5%, and most preferably within 3%, 2% or 1% of a full length polypeptide of the present disclosure.

Optionally, the polynucleotides of this embodiment will encode a protein having a specific enzymatic activity at least 50%, 60%, 80% or 90% of a cellular extract comprising the native, endogenous full-length polypeptide of the present disclosure. Further, the proteins encoded by polynucleotides of this embodiment will optionally have a substantially similar affinity constant (Kₘ) and/or catalytic activity (i.e., the microscopic rate constant, k_{cat}) as the native endogenous, full-length protein. Those of skill in the art will recognize that k_{cat}/Kₘ value determines the specificity for competing substrates and is often referred to as the specificity constant. Proteins of this embodiment can have a k_{cat}/Kₘ value at least 10% of a full-length polypeptide of the present disclosure as determined using the endogenous substrate of that polypeptide. Optionally, the k_{cat}/Kₘ value will be at least 20%, 30%, 40%, 50% and most preferably at least 60%, 70%, 80%, 90% or 95% the k_{cat}/Kₘ value of the full-length polypeptide of the present disclosure. Determination of k_{cat}, Kₘ, and k_{cat}/Kₘ can be determined by any number of means well known to those of skill in the art. For example, the initial rates (i.e., the first 5% or less of the reaction) can be determined using rapid mixing and sampling techniques (e.g., continuous-flow, stopped-flow or rapid quenching techniques), flash photolysis or relaxation methods (e.g., temperature jumps) in conjunction with such exemplary methods of measuring as spectrophotometry, spectrofluorimetry, nuclear magnetic resonance or radioactive procedures. Kinetic values are conveniently obtained using a Lineweaver-Burk or Eadie-Hofstee plot.

### F. Polynucleotides Complementary to the Polynucleotides of (A)-(E)

As indicated in (f), above, the present disclosure provides isolated nucleic acids comprising polynucleotides complementary to the polynucleotides of paragraphs A-E, above. As those of skill in the art will recognize, complementary sequences base-pair throughout the entirety of their length with the polynucleotides of sections (A)-(E) (i.e., have 100% sequence identity over their entire length). Complementary bases associate through hydrogen bonding in double stranded nucleic acids. For example, the following base pairs are complementary: guanine and cytosine; adenine and thymine and adenine and uracil.

G. Polynucleotides which are Subsequences of the Polynucleotides of (A)-(F)

As indicated in (g), above, the present disclosure provides isolated nucleic acids comprising polynucleotides which comprise at least 15 contiguous bases from the polynucleotides of sections (A) through (F) as discussed above. The length of the polynucleotide is given as an integer selected from the group consisting of from at least 15 to the length of the nucleic acid sequence from which the polynucleotide is a subsequence of. Thus, for example, polynucleotides of the present disclosure are inclusive of polynucleotides comprising at least 15, 20, 25, 30, 40, 50, 60, 75 or 100 contiguous nucleotides in length from the polynucleotides of (A)-(F). Optionally, the number of such subsequences encoded by a polynucleotide of the instant embodiment can be any integer selected from the group consisting of from 1 to 20, such as 2, 3, 4 or 5. The subsequences can be separated by any integer of nucleotides from 1 to the number of nucleotides in the sequence such as at least 5, 10, 15, 25, 50, 100 or 200 nucleotides.

Subsequences can be made by in vitro synthetic, in vitro biosynthetic or in vivo recombinant methods. In optional embodiments, subsequences can be made by nucleic acid amplification. For example, nucleic acid primers will be constructed to selectively hybridize to a sequence (or its complement) within, or co-extensive with, the coding region.

The subsequences of the present disclosure can comprise structural characteristics of the sequence from which it is derived. Alternatively, the subsequences can lack certain structural characteristics of the larger sequence from which it is derived such as a poly (A) tail. Optionally, a subsequence from a polynucleotide encoding a polypeptide having at least one epitope in common with a prototype polypeptide sequence as provided in (a), above, may encode an epitope in common with the prototype sequence. Alternatively, the subsequence may not encode an epitope in common with the prototype sequence but can be used to isolate the larger sequence by, for example, nucleic acid hybridization with the sequence from which it's derived. Subsequences can be used to modulate or detect gene expression by introducing into the subsequences compounds which bind, intercalate, cleave and/or crosslink to nucleic acids. Exemplary compounds include acridine, psoralen, phenanthroline, naphthoquinone, daunomycin or chloroethylaminoaryl conjugates.

H. Polynucleotides from a Full-Length Enriched cDNA Library Having the Physico-Chemical Property of Selectively Hybridizing to a Polynucleotide of (A)-(G)

As indicated in (h), above, the present disclosure provides an isolated polynucleotide from a full-length enriched cDNA library having the physico-chemical property of selectively hybridizing to a polynucleotide of paragraphs (A), (B), (C), (D), (E), (F) or (G) as discussed above. Methods of constructing full-length enriched cDNA libraries are known in the art and discussed briefly below. The cDNA library comprises at least 50% to 95% full-length sequences (for example, at least 50%, 60%, 70%, 80%, 90% or 95% full-length sequences). The cDNA library can be constructed from a variety of tissues from a monocot or dicot at a variety of developmental stages. Exemplary species include maize, wheat, rice, canola, soybean, cotton, sorghum, sunflower, alfalfa, oats, sugar cane, millet, barley and rice. Methods of selectively hybridizing, under selective hybridization conditions, a polynucleotide from a full-length enriched library to a polynucleotide of the present disclosure are known to those of ordinary skill in the art. Any number of stringency conditions can be employed to allow for selective hybridization. In optional embodiments, the stringency allows for selective hybridization of sequences having at least 70%, 75%, 80%, 85%, 90%, 95% or 98% sequence identity over the length of the hybridized region. Full-length enriched cDNA libraries can be normalized to increase the representation of rare sequences.

I Polynucleotide Products Made by a cDNA Isolation Process

As indicated in (I), above, the present disclosure provides an isolated polynucleotide made by the process of: 1) providing a full-length enriched nucleic acid library, 2) selectively hybridizing the polynucleotide to a polynucleotide of paragraphs (A), (B), (C), (D), (E), (F), (G) or (H) as discussed above, and thereby isolating the polynucleotide from the nucleic acid library. Full-length enriched nucleic acid libraries are constructed as discussed in paragraph (G) and below. Selective hybridization conditions are as discussed in paragraph (G). Nucleic acid purification procedures are well known in the art. Purification can be conveniently accomplished using solid-phase methods; such methods are well known to those of skill in the art and kits are available from commercial suppliers such as Advanced Biotechnologies (Surrey, UK). For example, a polynucleotide of paragraphs (A)-(H) can be immobilized to a solid support such as a membrane, bead, or particle. See, e.g., U.S. Pat. No. 5,667,976. The polynucleotide product of the present process is selectively hybridized to an immobilized polynucleotide and the solid support is subsequently isolated from non-hybridized polynucleotides by methods including, but not limited to, centrifugation, magnetic separation, filtration, electrophoresis and the like.

### Construction of Nucleic Acids

The isolated nucleic acids of the present disclosure can be made using (a) standard recombinant methods, (b) synthetic techniques or combinations thereof. In some embodiments, the polynucleotides of the present disclosure will be cloned, amplified or otherwise constructed from a monocot such as maize, rice or wheat or a dicot such as soybean.

The nucleic acids may conveniently comprise sequences in addition to a polynucleotide of the present disclosure. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present disclosure. For example, a hexa-histidine marker sequence provides a convenient means to purify the proteins of the present disclosure. A polynucleotide of the present disclosure can be attached to a vector, adapter or linker for cloning and/or expression of a polynucleotide of the present disclosure. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present disclosure less the length of its polynucleotide of the present disclosure is less than 20 kilobase pairs, often less than 15 kb and frequently less than 10 kb. Use of cloning vectors, expression vectors, adapters, and linkers is well known and extensively described in the art. For a description of various nucleic acids see, for example, Stratagene Cloning Systems, Catalogs 1999 (La Jolla, Calif.) and Amersham Life Sciences, Inc, Catalog '99 (Arlington Heights, Ill.).

### A. Recombinant Methods for Constructing Nucleic Acids

The isolated nucleic acid compositions of this disclosure, such as RNA, cDNA, genomic DNA or a hybrid thereof, can be obtained from plant biological sources using any number of cloning methodologies known to those of skill in the art. In some embodiments, oligonucleotide probes which selectively hybridize, under stringent conditions, to the polynucleotides of the present disclosure are used to identify the desired sequence in a cDNA or genomic DNA library. Isolation of RNA and construction of cDNA and genomic libraries is well known to those of ordinary skill in the art. See, e.g., Plant Molecular Biology: A Laboratory Manual, Clark, Ed., Springer-Verlag, Berlin (1997) and, Current Protocols in Molecular Biology, Ausubel, et al., Eds., Greene Publishing and Wiley-Interscience, New York (1995).

A1. Full-Length Enriched cDNA Libraries

A number of cDNA synthesis protocols have been described which provide enriched full-length cDNA libraries. Enriched full-length cDNA libraries are constructed to comprise at least 600%, and more preferably at least 70%, 80%, 90% or 95% full-length inserts amongst clones containing inserts. The length of insert in such libraries can be at least 2, 3, 4, 5, 6, 7, 8, 9, 10 or more kilobase pairs. Vectors to accommodate inserts of these sizes are known in the art and available commercially. See, e.g., Stratagene's lambda ZAP Express (cDNA cloning vector with 0 to 12 kb cloning capacity). An exemplary method of constructing a greater than 95% pure full-length cDNA library is described by Carninci, et al., (1996) Genomics, 37:327-336. Other methods for producing full-length libraries are known in the art. See, e.g., Edery, et al., (1995) Mol. Cell Biol. 15(6):3363-3371 and PCT Application Number WO 1996/34981.

A2 Normalized or Subtracted cDNA Libraries

A non-normalized cDNA library represents the mRNA population of the tissue it was made from. Since unique clones are out-numbered by clones derived from highly expressed genes their isolation can be laborious. Normalization of a cDNA library is the process of creating a library in which each clone is more equally represented. Construction of normalized libraries is described in Ko, (1990) Nucl. Acids. Res. 18(19):5705-5711; Patanjali, et al., (1991) Proc. Natl. Acad. U.S.A. 88:1943-1947; U.S. Pat. Nos. 5,482,685, 5,482,845 and 5,637,685. In an exemplary method described by Soares, et al., normalization resulted in reduction of the abundance of clones from a range of four orders of magnitude to a narrow range of only 1 order of magnitude. Proc. Natl. Acad. Sci. USA, 91:9228-9232 (1994).

Subtracted cDNA libraries are another means to increase the proportion of less abundant cDNA species. In this procedure, cDNA prepared from one pool of mRNA is depleted of sequences present in a second pool of mRNA by hybridization. The cDNA:mRNA hybrids are removed and the remaining un-hybridized cDNA pool is enriched for sequences unique to that pool. See, Foote, et al., in, Plant Molecular Biology: A Laboratory Manual, Clark, Ed., Springer-Verlag, Berlin (1997); Kho and Zarbl, (1991) Technique 3(2):58-63; Sive and St. John, (1988) Nucl. Acids Res., 16(22):10937; Current Protocols in Molecular Biology, Ausubel, et al., Eds., Greene Publishing and Wiley-Interscience, New York (1995) and Swaroop, et al., (1991) Nucl. Acids Res., 19(8):1954. cDNA subtraction kits are commercially available. See, e.g., PCR-Select (Clontech, Palo Alto, Calif.).

To construct genomic libraries, large segments of genomic DNA are generated by fragmentation, e.g., using restriction endonucleases, and are ligated with vector DNA to form concatemers that can be packaged into the appropriate vector. Methodologies to accomplish these ends and sequencing methods to verify the sequence of nucleic acids are well known in the art. Examples of appropriate molecular biological techniques and instructions sufficient to direct persons of skill through many construction, cloning and screening methodologies are found in Sambrook, et al., Molecular Cloning A Laboratory Manual, 2nd Ed., Cold Spring Harbor Laboratory Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel, et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a polynucleotide of the present disclosure such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent.

The nucleic acids of interest can also be amplified from nucleic acid samples using amplification techniques. For instance, polymerase chain reaction (PCR) technology can be used to amplify the sequences of polynucleotides of the present disclosure and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing or for other purposes. The T4 gene 32 protein (Boehringer Mannheim) can be used to improve yield of long PCR products.

PCR-based screening methods have been described. Wilfinger, et al., describe a PCR-based method in which the longest cDNA is identified in the first step so that incomplete clones can be eliminated from study. BioTechniques 22(3):481-486 (1997). Such methods are particularly effective in combination with a full-length cDNA construction methodology, above.

### B. Synthetic Methods for Constructing Nucleic Acids

The isolated nucleic acids of the present disclosure can also be prepared by direct chemical synthesis by methods such as the phosphotriester method of Narang, et al., (1979) Meth. Enzymol. 68: 90-99; the phosphodiester method of Brown, et al., (1979) Meth. Enzymol. 68:109-151; the diethylphosphoramidite method of Beaucage, et al., (1981) Tetra. Lett. 22:1859-1862; the solid phase phosphoramidite triester method described by Beaucage and Caruthers, (1981) Tetra. Letts. 22(20):1859-1862, e.g., using an automated synthesizer, e.g., as described in Needham-VanDevanter, et al., (1984) Nucleic Acids Res., 12:6159-6168 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is best employed for sequences of about 100 bases or less, longer sequences may be obtained by the ligation of shorter sequences.

### Recombinant Expression Cassettes

The present disclosure further provides recombinant expression cassettes comprising a nucleic acid of the present disclosure. A nucleic acid sequence coding for the desired polypeptide of the present disclosure, for example a cDNA or a genomic sequence encoding a full length polypeptide of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter and the GRP1-8 promoter.

Alternatively, the plant promoter can direct expression of a polynucleotide of the present disclosure in a specific tissue or may be otherwise under more precise environmental or developmental control. Such promoters are referred to here as “inducible” promoters. Environmental conditions that may affect transcription by inducible promoters include pathogen attack, anaerobic conditions or the presence of light. Examples of inducible promoters are the Adh1 promoter which is inducible by hypoxia or cold stress, the Hsp70 promoter which is inducible by heat stress and the PPDK promoter which is inducible by light.

Examples of promoters under developmental control include promoters that initiate transcription only, or preferentially, in certain tissues, such as leaves, roots, fruit, seeds or flowers. Exemplary promoters include the anther-specific promoter 5126 (U.S. Pat. Nos. 5,689,049 and 5,689,051), glb-1 promoter and gamma-zein promoter. Also see, for example, US Patent Application Ser. Nos. 60/155,859 and 60/163,114. The operation of a promoter may also vary depending on its location in the genome. Thus, an inducible promoter may become fully or partially constitutive in certain locations.

Both heterologous and non-heterologous (i.e., endogenous) promoters can be employed to direct expression of the nucleic acids of the present disclosure. These promoters can also be used, for example, in recombinant expression cassettes to drive expression of antisense nucleic acids to reduce, increase or alter concentration and/or composition of the proteins of the present disclosure in a desired tissue. Thus, in some embodiments, the nucleic acid construct will comprise a promoter, functional in a plant cell, operably linked to a polynucleotide of the present disclosure. Promoters useful in these embodiments include the endogenous promoters driving expression of a polypeptide of the present disclosure.

In some embodiments, isolated nucleic acids which serve as promoter or enhancer elements can be introduced in the appropriate position (generally upstream) of a non-heterologous form of a polynucleotide of the present disclosure so as to up or down regulate expression of a polynucleotide of the present disclosure. For example, endogenous promoters can be altered in vivo by mutation, deletion and/or substitution (see, Kmiec, U.S. Pat. No. 5,565,350; Zarling, et al., PCT/US93/03868) or isolated promoters can be introduced into a plant cell in the proper orientation and distance from a cognate gene of a polynucleotide of the present disclosure so as to control the expression of the gene. Gene expression can be modulated under conditions suitable for plant growth so as to alter the total concentration and/or alter the composition of the polypeptides of the present disclosure in plant cell. Thus, the present disclosure provides compositions, and methods for making, heterologous promoters and/or enhancers operably linked to a native, endogenous (i.e., non-heterologous) form of a polynucleotide of the present disclosure.

If polypeptide expression is desired, it is generally desirable to include a polyadenylation region at the 3′-end of a polynucleotide coding region. The polyadenylation region can be derived from the natural gene, from a variety of other plant genes or from T-DNA. The 3′ end sequence to be added can be derived from, for example, the nopaline synthase or octopine synthase genes or alternatively from another plant gene or less preferably from any other eukaryotic gene.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, (1988) Mol. Cell Biol. 8:4395-4405; Callis, et al., (1987) Genes Dev. 1:11831200. Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot, Eds., Springer, New York (1994). The vector comprising the sequences from a polynucleotide of the present disclosure will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers, et al., (1987) Meth. in Enzymol. 153:253-277.

A polynucleotide of the present disclosure can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy, et al., (1988) Proc. Nat'l. Acad. Sci. (USA) 85:8805-8809 and Hiatt, et al., U.S. Pat. No. 4,801,340.

Another method of suppression is sense suppression (i.e., co-supression). Introduction of nucleic acid configured in the sense orientation has been shown to be an effective means by which to block the transcription of target genes. For an example of the use of this method to modulate expression of endogenous genes see, Napoli, et al., (1990) The Plant Cell 2:279-289 and U.S. Pat. No. 5,034,323.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff, et al., (1988) Nature 334:585-591.

A variety of cross-linking agents, alkylating agents and radical generating species as pendant groups on polynucleotides of the present disclosure can be used to bind, label, detect and/or cleave nucleic acids. For example, Vlassov, et al., (1986) Nucleic Acids Res 14:4065-4076, describe covalent bonding of a single-stranded DNA fragment with alkylating derivatives of nucleotides complementary to target sequences. A report of similar work by the same group is that by Knorre, et al., (1985) Biochimie 67:785-789. Iverson and Dervan also showed sequence-specific cleavage of single-stranded DNA mediated by incorporation of a modified nucleotide which was capable of activating cleavage (J Am Chem Soc (1987) 109:1241-1243). Meyer, et al., (1989) J Am Chem Soc 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, et al., (1988) Biochemistry 27:3197-3203. Use of crosslinking in triple-helix forming probes was also disclosed by Home, et al., (1990) J Am Chem Soc 112:2435-2437. Use of N4,N4-ethanocytosine as an alkylating agent to crosslink to single-stranded oligonucleotides has also been described by Webb and Matteucci, (1986) J Am Chem Soc 108:2764-2765; Nucleic Acids Res (1986) 14:7661-7674; Feteritz, et al., (1991) J. Am. Chem. Soc. 113:4000. Various compounds to bind, detect, label, and/or cleave nucleic acids are known in the art. See, for example, U.S. Pat. Nos. 5,543,507; 5,672,593; 5,484,908; 5,256,648 and 5,681941.

### Proteins

The isolated proteins of the present disclosure comprise a polypeptide having at least 10 amino acids from a polypeptide of the present disclosure (or conservative variants thereof) such as those encoded by any one of the polynucleotides of the present disclosure as discussed more fully above (e.g., Table 1). The proteins of the present disclosure or variants thereof can comprise any number of contiguous amino acid residues from a polypeptide of the present disclosure, wherein that number is selected from the group of integers consisting of from 10 to the number of residues in a full-length polypeptide of the present disclosure. Optionally, this subsequence of contiguous amino acids is at least 15, 20, 25, 30, 35 or 40 amino acids in length, often at least 50, 60, 70, 80 or 90 amino acids in length. Further, the number of such subsequences can be any integer selected from the group consisting of from 1 to 20, such as 2, 3, 4 or 5.

The present disclosure further provides a protein comprising a polypeptide having a specified sequence identity/similarity with a polypeptide of the present disclosure. The percentage of sequence identity/similarity is an integer selected from the group consisting of from 50 to 99. Exemplary sequence identity/similarity values include 55%, 60%, 65%, 70%, 75%, 80%, 85%, 90%, 95%, 96%, 97% 98% and 99%. Sequence identity can be determined using, for example, the GAP, CLUSTALW or BLAST algorithms.

As those of skill will appreciate, the present disclosure includes, but is not limited to, catalytically active polypeptides of the present disclosure (i.e., enzymes). Catalytically active polypeptides have a specific activity of at least 20%, 30% or 40% and preferably at least 50%, 60% or 70% and most preferably at least 80%, 90% or 95% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, or 50%, that of the native (non-synthetic), endogenous polypeptide and more preferably at least 60%, 70%, 80% 85%, 90%, 95%, 96%, 97% 98% or 99%. Methods of assaying and quantifying measures of enzymatic activity and substrate specificity (k_{cat}/Kₘ) are well known to those of skill in the art.

Generally, the proteins of the present disclosure will, when presented as an immunogen, elicit production of an antibody specifically reactive to a polypeptide of the present disclosure. Further, the proteins of the present disclosure will not bind to antisera raised against a polypeptide of the present disclosure which has been fully immunosorbed with the same polypeptide. Immunoassays for determining binding are well known to those of skill in the art. A preferred immunoassay is a competitive immunoassay. Thus, the proteins of the present disclosure can be employed as immunogens for constructing antibodies immunoreactive to a protein of the present disclosure for such exemplary utilities as immunoassays or protein purification techniques.

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present disclosure. To obtain high level expression of a cloned gene, it is desirable to construct expression vectors which contain, at the minimum, a strong promoter to direct transcription, a ribosome binding site for translational initiation and a transcription/translation terminator. One of skill would recognize that modifications can be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located purification sequences. Restriction sites or termination codons can also be introduced.

### Synthesis of Proteins

The proteins of the present disclosure can be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology Vol. 2: Special Methods in Peptide Synthesis, Part A.; Merrifield, et al., (1963) J. Am. Chem. Soc. 85:2149-2156 and Stewart, et al., Solid Phase Peptide Synthesis, 2nd ed., Pierce Chem. Co., Rockford, III. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) are known to those of skill.

### Purification of Proteins

The proteins of the present disclosure may be purified by standard techniques well known to those of skill in the art. Recombinantly produced proteins of the present disclosure can be directly expressed or expressed as a fusion protein. The recombinant protein is purified by a combination of cell lysis (e.g., sonication, French press) and affinity chromatography. For fusion products, subsequent digestion of the fusion protein with an appropriate proteolytic enzyme releases the desired recombinant protein.

The proteins of this disclosure, recombinant or synthetic, may be purified to substantial purity by standard techniques well known in the art, including detergent solubilization, selective precipitation with such substances as ammonium sulfate, column chromatography, immunopurification methods and others. See, for instance, Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

Introduction of Nucleic Acids into Host Cells

The method of introducing a nucleic acid of the present disclosure into a host cell is not critical to the instant disclosure. Transformation or transfection methods are conveniently used. Accordingly, a wide variety of methods have been developed to insert a DNA sequence into the genome of a host cell to obtain the transcription and/or translation of the sequence to effect phenotypic changes in the organism. Thus, any method which provides for effective introduction of a nucleic acid may be employed.

### A. Plant Transformation

A nucleic acid comprising a polynucleotide of the present disclosure is optionally introduced into a plant. Generally, the polynucleotide will first be incorporated into a recombinant expression cassette or vector. Isolated nucleic acid acids of the present disclosure can be introduced into plants according to techniques known in the art. Techniques for transforming a wide variety of higher plant species are well known and described in the technical, scientific, and patent literature. See, for example, Weising, et al., (1988) Ann. Rev. Genet. 22:421-477. For example, the DNA construct may be introduced directly into the genomic DNA of the plant cell using techniques such as electroporation, polyethylene glycol (PEG) poration, particle bombardment, silicon fiber delivery or microinjection of plant cell protoplasts or embryogenic callus. See, e.g., Tomes, et al., Direct DNA Transfer into Intact Plant Cells Via Microprojectile Bombardment. pp. 197-213 in Plant Cell, Tissue and Organ Culture, Fundamental Methods. eds. Gamborg and Phillips. Springer-Verlag Berlin Heidelberg New York, 1995; see, U.S. Pat. No. 5,990,387. The introduction of DNA constructs using PEG precipitation is described in Paszkowski, et al., (1984) Embo J. 3:2717-2722. Electroporation techniques are described in Fromm, et al., (1985) Proc. Natl. Acad. Sci. (USA) 82:5824. Ballistic transformation techniques are described in Klein, et al., (1987) Nature 327:70-73.

Agrobacterium tumefaciens-mediated transformation techniques are well described in the scientific literature. See, for example, Horsch, et al., (1984) Science 233:496-498; Fraley, et al., (1983) Proc. Natl. Acad. Sci. (USA) 80:4803 and Plant Molecular Biology: A Laboratory Manual, Chapter 8, Clark, Ed., Springer-Verlag, Berlin (1997). The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include (1) Agrobacterium rhizogenes-mediated transformation (see, e.g., Lichtenstein and Fuller In: Genetic Engineering, vol. 6, Rigby, Ed., London, Academic Press, 1987; and Lichtenstein, and Draper, In: DNA Cloning, Vol. II, Glover, Ed., Oxford, IRI Press, 1985), PCT Application Number PCT/US87/02512 (WO 1988/02405 published Apr. 7, 1988) describes the use of A. rhizogenes strain A4 and its Ri plasmid along with A. tumefaciens vectors pARC8 or pARC16 (2) liposome-mediated DNA uptake (see, e.g., Freeman, et al., (1984) Plant Cell Physiol. 25:1353), (3) the vortexing method (see, e.g., Kindle, (1990) Proc. Natl. Acad. Sci., (USA) 87:1228).

DNA can also be introduced into plants by direct DNA transfer into pollen as described by Zhou, et al., (1983) Methods in Enzymology 101:433; Hess, (1987) Intern Rev. Cytol. 107:367; Luo, et al., (1988) Plant Mol. Biol. Reporter 6:165. Expression of polypeptide coding genes can be obtained by injection of the DNA into reproductive organs of a plant as described by Pena, et al., (2007) Plant Cell 19:549-563. DNA can also be injected directly into the cells of immature embryos and the rehydration of desiccated embryos as described by Neuhaus, et al., (1987) Theor. Appl. Genet., 75:30 and Benbrook, et al., in Proceedings Bio Expo 1986, Butterworth, Stoneham, Mass., pp. 27-54 (1986). A variety of plant viruses that can be employed as vectors are known in the art and include cauliflower mosaic virus (CaMV), geminivirus, brome mosaic virus, and tobacco mosaic virus.

### B. Transfection of Prokaryotes, Lower Eukaryotes, and Animal Cells

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics and micro-injection of the DNA directly into the cells. The transfected cells are cultured by means well known in the art. Kuchler, Biochemical Methods in Cell Culture and Virology, Dowden, Hutchinson and Ross, Inc. (1977).

### Transgenic Plant Regeneration

Plant cells which directly result or are derived from the nucleic acid introduction techniques can be cultured to regenerate a whole plant which possesses the introduced genotype. Such regeneration techniques often rely on manipulation of certain phytohormones in a tissue culture growth medium. Plants cells can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans, et al., Protoplasts Isolation and Culture, Handbook of Plant Cell Culture, Macmillan Publishing Company, New York, pp. 124-176 (1983) and Binding, Regeneration of Plants, Plant Protoplasts, CRC Press, Boca Raton, pp. 21-73 (1985).

The regeneration of plants from either single plant protoplasts or various explants is well known in the art. See, for example, Methods for Plant Molecular Biology, Weissbach and Weissbach, eds., Academic Press, Inc., San Diego, Calif. (1988). This regeneration and growth process includes the steps of selection of transformant cells and shoots, rooting the transformant shoots and growth of the plantlets in soil. For maize cell culture and regeneration see generally, The Maize Handbook, Freeling and Walbot, Eds., Springer, New York (1994); Corn and Corn Improvement, 3^{rd} edition, Sprague and Dudley Eds., American Society of Agronomy, Madison, Wis. (1988). For transformation and regeneration of maize see, Gordon-Kamm, et al., (1990) The Plant Cell 2:603-618.

The regeneration of plants containing the polynucleotide of the present disclosure and introduced by Agrobacterium from leaf explants can be achieved as described by Horsch, et al., (1985) Science, 227:1229-1231. In this procedure, transformants are grown in the presence of a selection agent and in a medium that induces the regeneration of shoots in the plant species being transformed as described by Fraley, et al., (1983) Proc. Natl. Acad. Sci. (U.S.A.) 80:4803. This procedure typically produces shoots within two to four weeks and these transformant shoots are then transferred to an appropriate root-inducing medium containing the selective agent and an antibiotic to prevent bacterial growth. Transgenic plants of the present disclosure may be fertile or sterile.

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit and the like are included in the disclosure, provided that these parts comprise cells comprising the isolated nucleic acid of the present disclosure. Progeny and variants, and mutants of the regenerated plants are also included within the scope of the disclosure, provided that these parts comprise the introduced nucleic acid sequences.

Transgenic plants expressing a polynucleotide of the present disclosure can be screened for transmission of the nucleic acid of the present disclosure by, for example, standard immunoblot and DNA detection techniques. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then analyzed for protein expression by Western immunoblot analysis using the specifically reactive antibodies of the present disclosure. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present disclosure relative to a control plant (i.e., native, non-transgenic). Back-crossing to a parental plant and out-crossing with a non-transgenic plant are also contemplated.

Modulating Polypeptide Levels and/or Composition

The present disclosure further provides a method for modulating (i.e., increasing or decreasing) the concentration or ratio of the polypeptides of the present disclosure in a plant or part thereof. Modulation can be effected by increasing or decreasing the concentration and/or the ratio of the polypeptides of the present disclosure in a plant. The method comprises introducing into a plant cell a recombinant expression cassette comprising a polynucleotide of the present disclosure as described above to obtain a transgenic plant cell, culturing the transgenic plant cell under transgenic plant cell growing conditions and inducing or repressing expression of a polynucleotide of the present disclosure in the transgenic plant for a time sufficient to modulate concentration and/or the ratios of the polypeptides in the transgenic plant or plant part.

In some embodiments, the concentration and/or ratios of polypeptides of the present disclosure in a plant may be modulated by altering, in vivo or in vitro, the promoter of a gene to up- or down-regulate gene expression. In some embodiments, the coding regions of native genes of the present disclosure can be altered via substitution, addition, insertion or deletion to decrease activity of the encoded enzyme. (See, e.g., Kmiec, U.S. Pat. No. 5,565,350; Zarling, et al., PCT/US93/03868.) And in some embodiments, an isolated nucleic acid (e.g., a vector) comprising a promoter sequence is transfected into a plant cell. Subsequently, a plant cell comprising the promoter operably linked to a polynucleotide of the present disclosure is selected for by means known to those of skill in the art such as, but not limited to, Southern blot, DNA sequencing or PCR analysis using primers specific to the promoter and to the gene and detecting amplicons produced therefrom. A plant or plant part altered or modified by the foregoing embodiments is grown under plant forming conditions for a time sufficient to modulate the concentration and/or ratios of polypeptides of the present disclosure in the plant. Plant forming conditions are well known in the art and discussed briefly, supra.

In general, concentration or the ratios of the polypeptides is increased or decreased by at least 5%, 10%, 20%, 30%, 40%, 50%, 60%, 70%, 80% or 90% relative to a native control plant, plant part, or cell lacking the aforementioned recombinant expression cassette. Modulation in the present disclosure may occur during and/or subsequent to growth of the plant to the desired stage of development. Modulating nucleic acid expression temporally and/or in particular tissues can be controlled by employing the appropriate promoter operably linked to a polynucleotide of the present disclosure in, for example, sense or antisense orientation as discussed in greater detail, supra. Induction of expression of a polynucleotide of the present disclosure can also be controlled by exogenous administration of an effective amount of inducing compound. Inducible promoters and inducing compounds which activate expression from these promoters are well known in the art. In preferred embodiments, the polypeptides of the present disclosure are modulated in monocots, particularly maize.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 7-methylguanosine cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ untranslated regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host such as to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group (see, Devereaux, et al., (1984) Nucleic Acids Res. 12:387-395) or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides that can be used to determine a codon usage frequency can be any integer from 1 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number WO 1997/20078. See also, Zhang, et al., (1997) Proc. Natl. Acad. Sci. USA 94:4504-4509. Generally, sequence shuffling provides a means for generating libraries of polynucleotides having a desired characteristic which can be selected or screened for. Libraries of recombinant polynucleotides are generated from a population of related sequence polynucleotides which comprise sequence regions which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system and may include properties of: an encoded protein, a transcriptional element, a sequence controlling transcription, RNA processing, RNA stability, chromatin conformation, translation, or other expression property of a gene or transgene, a replicative element, a protein-binding element or the like, such as any feature which confers a selectable or detectable property. In some embodiments, the selected characteristic will be a decreased Kₘ and/or increased K_{cat} over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140% or at least 150% of the wild-type value.

### Generic and Consensus Sequences

Polynucleotides and polypeptides of the present disclosure further include those having: (a) a generic sequence of at least two homologous polynucleotides or polypeptides, respectively, of the present disclosure and (b) a consensus sequence of at least three homologous polynucleotides or polypeptides, respectively, of the present disclosure. The generic sequence of the present disclosure comprises each species of polypeptide or polynucleotide embraced by the generic polypeptide or polynucleotide sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phyla or kingdoms. For example, a polynucleotide having a consensus sequence from a gene family of Zea mays can be used to generate antibody or nucleic acid probes or primers to other Gramineae species such as wheat, rice or sorghum. Alternatively, a polynucleotide having a consensus sequence generated from orthologous genes can be used to identify or isolate orthologs of other taxa. Typically, a polynucleotide having a consensus sequence will be at least 9, 10, 15, 20, 25, 30 or 40 amino acids in length, or 20, 30, 40, 50, 100 or 150 nucleotides in length. As those of skill in the art are aware, a conservative amino acid substitution can be used for amino acids which differ amongst aligned sequence but are from the same conservative substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous or paralogous sequences as provided herein. Optionally, similar sequences used in generating a consensus or generic sequence are identified using the BLAST algorithm's smallest sum probability (P(N)). Various suppliers of sequence-analysis software are listed in chapter 7 of Current Protocols in Molecular Biology, Ausubel et al., Eds., Current Protocols, a joint venture between Greene Publishing Associates, Inc. and John Wiley & Sons, Inc. (Supplement 30). A polynucleotide sequence is considered similar to a reference sequence if the smallest sum probability in a comparison of the test nucleic acid to the reference nucleic acid is less than about 0.1, more preferably less than about 0.01, or 0.001 and most preferably less than about 0.0001 or 0.00001. Similar polynucleotides can be aligned and a consensus or generic sequence generated using multiple sequence alignment software available from a number of commercial suppliers such as the Genetics Computer Group's (Madison, Wis.) PILEUP software, Vector NTI's (North Bethesda, Md.) ALIGNX, or Genecode's (Ann Arbor, Mich.) SEQUENCHER. Conveniently, default parameters of such software can be used to generate consensus or generic sequences.

### Machine Applications

The present disclosure provides machines, data structures, and processes for modeling or analyzing the polynucleotides and polypeptides of the present disclosure.

### A. Machines: Data, Data Structures, Processes and Functions

The present disclosure provides a machine having a memory comprising: 1) data representing a sequence of a polynucleotide or polypeptide of the present disclosure, 2) a data structure which reflects the underlying organization and structure of the data and facilitates program access to data elements corresponding to logical sub-components of the sequence, 3) processes for effecting the use, analysis, or modeling of the sequence and 4) optionally, a function or utility for the polynucleotide or polypeptide. Thus, the present disclosure provides a memory for storing data that can be accessed by a computer programmed to implement a process for affecting the use, analyses or modeling of a sequence of a polynucleotide, with the memory comprising data representing the sequence of a polynucleotide of the present disclosure.

The machine of the present disclosure is typically a digital computer. The term “computer” includes one or several desktop or portable computers, computer workstations, servers (including intranet or internet servers), mainframes and any integrated system comprising any of the above irrespective of whether the processing, memory, input or output of the computer is remote or local, as well as any networking interconnecting the modules of the computer. The term “computer” is exclusive of computers of the United States Patent and Trademark Office or the European Patent Office when data representing the sequence of polypeptides or polynucleotides of the present disclosure is used for patentability searches.

The present disclosure contemplates providing as data a sequence of a polynucleotide of the present disclosure embodied in a computer readable medium. As those of skill in the art will be aware, the form of memory of a machine of the present disclosure or the particular embodiment of the computer readable medium, are not critical elements of the disclosure and can take a variety of forms. The memory of such a machine includes, but is not limited to, ROM or RAM or computer readable media such as, but not limited to, magnetic media such as computer disks or hard drives or media such as CD-ROMs, DVDs and the like.

The present disclosure further contemplates providing a data structure that is also contained in memory. The data structure may be defined by the computer programs that define the processes (see below) or it may be defined by the programming of separate data storage and retrieval programs subroutines or systems. Thus, the present disclosure provides a memory for storing a data structure that can be accessed by a computer programmed to implement a process for affecting the use, analysis or modeling of a sequence of a polynucleotide. The memory comprises data representing a polynucleotide having the sequence of a polynucleotide of the present disclosure. The data is stored within memory. Further, a data structure, stored within memory, is associated with the data reflecting the underlying organization and structure of the data to facilitate program access to data elements corresponding to logical sub-components of the sequence. The data structure enables the polynucleotide to be identified and manipulated by such programs.

In a further embodiment, the present disclosure provides a data structure that contains data representing a sequence of a polynucleotide of the present disclosure stored within a computer readable medium. The data structure is organized to reflect the logical structuring of the sequence, so that the sequence is easily analyzed by software programs capable of accessing the data structure. In particular, the data structures of the present disclosure organize the reference sequences of the present disclosure in a manner which allows software tools to perform a wide variety of analyses using logical elements and sub-elements of each sequence.

An example of such a data structure resembles a layered hash table, where in one dimension the base content of the sequence is represented by a string of elements A, T, C, G and N. The direction from the 5′ end to the 3′ end is reflected by the order from the position 0 to the position of the length of the string minus one. Such a string, corresponding to a nucleotide sequence of interest, has a certain number of substrings, each of which is delimited by the string position of its 5′ end and the string position of its 3′ end within the parent string. In a second dimension, each substring is associated with or pointed to one or multiple attribute fields. Such attribute fields contain annotations to the region on the nucleotide sequence represented by the substring.

For example, a sequence under investigation is 520 bases long and represented by a string named SeqTarget. There is a minor groove in the 5′ upstream non-coding region from position 12 to 38, which is identified as a binding site for an enhancer protein HM-A, which in turn will increase the transcription of the gene represented by SeqTarget. Here, the substring is represented as (12, 38) and has the following attributes: [upstream uncoded], [minor groove], [HM-A binding] and [increase transcription upon binding by HM-A]. Similarly, other types of information can be stored and structured in this manner, such as information related to the whole sequence, e.g., whether the sequence is a full length viral gene, a mammalian housekeeping gene or an EST from clone X, information related to the 3′ down stream non-coding region, e.g., hair pin structure and information related to various domains of the coding region, e.g., Zinc finger.

This data structure is an open structure and is robust enough to accommodate newly generated data and acquired knowledge. Such a structure is also a flexible structure. It can be trimmed down to a 1-D string to facilitate data mining and analysis steps, such as clustering, repeat-masking, and HMM analysis. Meanwhile, such a data structure also can extend the associated attributes into multiple dimensions. Pointers can be established among the dimensioned attributes when needed to facilitate data management and processing in a comprehensive genomics knowledgebase. Furthermore, such a data structure is object-oriented. Polymorphism can be represented by a family or class of sequence objects, each of which has an internal structure as discussed above. The common traits are abstracted and assigned to the parent object, whereas each child object represents a specific variant of the family or class. Such a data structure allows data to be efficiently retrieved, updated and integrated by the software applications associated with the sequence database and/or knowledgebase.

The present disclosure contemplates providing processes for effecting analysis and modeling, which are described in the following section.

Optionally, the present disclosure further contemplates that the machine of the present disclosure will embody in some manner a utility or function for the polynucleotide or polypeptide of the present disclosure. The function or utility of the polynucleotide or polypeptide can be a function or utility for the sequence data, per se, or of the tangible material. Exemplary function or utilities include the name (per International Union of Biochemistry and Molecular Biology rules of nomenclature) or function of the enzyme or protein represented by the polynucleotide or polypeptide of the present disclosure; the metabolic pathway of the protein represented by the polynucleotide or polypeptide of the present disclosure; the substrate or product or structural role of the protein represented by the polynucleotide or polypeptide of the present disclosure or the phenotype (e.g., an agronomic or pharmacological trait) affected by modulating expression or activity of the protein represented by the polynucleotide or polypeptide of the present disclosure.

### B. Computer Analysis and Modeling

The present disclosure provides a process of modeling and analyzing data representative of a polynucleotide or polypeptide sequence of the present disclosure. The process comprises entering sequence data of a polynucleotide or polypeptide of the present disclosure into a machine having a hardware or software sequence modeling and analysis system, developing data structures to facilitate access to the sequence data, manipulating the data to model or analyze the structure or activity of the polynucleotide or polypeptide and displaying the results of the modeling or analysis. Thus, the present disclosure provides a process for affecting the use, analysis or modeling of a polynucleotide sequence or its derived peptide sequence through use of a computer having a memory. The process comprises: 1) placing into the memory data representing a polynucleotide having the sequence of a polynucleotide of the present disclosure, developing within the memory a data structure associated with the data and reflecting the underlying organization and structure of the data to facilitate program access to data elements corresponding to logical sub-components of the sequence, 2) programming the computer with a program containing instructions sufficient to implement the process for effecting the use, analysis or modeling of the polynucleotide sequence or the peptide sequence and 3) executing the program on the computer while granting the program access to the data and to the data structure within the memory.

A variety of modeling and analytic tools are well known in the art and available commercially. Included amongst the modeling/analysis tools are methods to: 1) recognize overlapping sequences (e.g., from a sequencing project) with a polynucleotide of the present disclosure and create an alignment called a “contig”; 2) identify restriction enzyme sites of a polynucleotide of the present disclosure; 3) identify the products of a T1 ribonuclease digestion of a polynucleotide of the present disclosure; 4) identify PCR primers with minimal self-complementarity; 5) compute pairwise distances between sequences in an alignment, reconstruct phylogentic trees using distance methods and calculate the degree of divergence of two protein coding regions; 6) identify patterns such as coding regions, terminators, repeats and other consensus patterns in polynucleotides of the present disclosure; 7) identify RNA secondary structure; 8) identify sequence motifs, isoelectric point, secondary structure, hydrophobicity and antigenicity in polypeptides of the present disclosure; 9) translate polynucleotides of the present disclosure and backtranslate polypeptides of the present disclosure and 10) compare two protein or nucleic acid sequences and identifying points of similarity or dissimilarity between them.

The processes for effecting analysis and modeling can be produced independently or obtained from commercial suppliers. Exemplary analysis and modeling tools are provided in products such as InforMax's (Bethesda, Md.) Vector NTI Suite (Version 5.5), Intelligenetics' (Mountain View, Calif.) PC/Gene program and Genetics Computer Group's (Madison, Wis.) Wisconsin Package® (Version 10.0); these tools, and the functions they perform, (as provided and disclosed by the programs and accompanying literature) are incorporated herein by reference and are described in more detail in section C which follows.

Thus, in a further embodiment, the present disclosure provides a machine-readable media containing a computer program and data, comprising a program stored on the media containing instructions sufficient to implement a process for affecting the use, analysis or modeling of a representation of a polynucleotide or peptide sequence. The data stored on the media represents a sequence of a polynucleotide having the sequence of a polynucleotide of the present disclosure. The media also includes a data structure reflecting the underlying organization and structure of the data to facilitate program access to data elements corresponding to logical sub-components of the sequence, the data structure being inherent in the program and in the way in which the program organizes and accesses the data.

### C. Homology Searches

As an example of such a comparative analysis, the present disclosure provides a process of identifying a candidate homologue (i.e., an ortholog or paralog) of a polynucleotide or polypeptide of the present disclosure. The process comprises entering sequence data of a polynucleotide or polypeptide of the present disclosure into a machine having a hardware or software sequence analysis system, developing data structures to facilitate access to the sequence data, manipulating the data to analyze the structure the polynucleotide or polypeptide and displaying the results of the analysis. A candidate homologue has statistically significant probability of having the same biological function (e.g., catalyzes the same reaction, binds to homologous proteins/nucleic acids, has a similar structural role) as the reference sequence to which it is compared. Accordingly, the polynucleotides and polypeptides of the present disclosure have utility in identifying homologs in animals or other plant species, particularly those in the family Gramineae such as, but not limited to, sorghum, wheat or rice.

The process of the present disclosure comprises obtaining data representing a polynucleotide or polypeptide test sequence. Test sequences can be obtained from a nucleic acid of an animal or plant. Test sequences can be obtained directly or indirectly from sequence databases including, but not limited to, those such as: GenBank, EMBL, GenSeq, SWISS-PROT or those available on-line via the UK Human Genome Mapping Project (HGMP) GenomeWeb. In some embodiments the test sequence is obtained from a plant species other than maize whose function is uncertain but will be compared to the test sequence to determine sequence similarity or sequence identity. The test sequence data is entered into a machine, such as a computer, containing: i) data representing a reference sequence and ii) a hardware or software sequence comparison system to compare the reference and test sequence for sequence similarity or identity.

Exemplary sequence comparison systems are provided for in sequence analysis software such as those provided by the Genetics Computer Group (Madison, Wis.) or InforMax (Bethesda, Md.) or Intelligenetics (Mountain View, Calif.). Optionally, sequence comparison is established using the BLAST or GAP suite of programs. Generally, a smallest sum probability value (P(N)) of less than 0.1, or alternatively, less than 0.01, 0.001, 0.0001 or 0.00001 using the BLAST 2.0 suite of algorithms under default parameters identifies the test sequence as a candidate homologue (i.e., an allele, ortholog or paralog) of the reference sequence. Those of skill in the art will recognize that a candidate homologue has an increased statistical probability of having the same or similar function as the gene/protein represented by the test sequence.

The reference sequence can be the sequence of a polypeptide or a polynucleotide of the present disclosure. The reference or test sequence is each optionally at least 25 amino acids or at least 100 nucleotides in length. The length of the reference or test sequences can be the length of the polynucleotide or polypeptide described, respectively, above in the sections entitled “Nucleic Acids” (particularly section (g)) and “Proteins”. As those of skill in the art are aware, the greater the sequence identity/similarity between a reference sequence of known function and a test sequence, the greater the probability that the test sequence will have the same or similar function as the reference sequence. The results of the comparison between the test and reference sequences are outputted (e.g., displayed, printed, recorded) via any one of a number of output devices and/or media (e.g., computer monitor, hard copy or computer readable medium).

### Detection of Nucleic Acids

The present disclosure further provides methods for detecting a polynucleotide of the present disclosure in a nucleic acid sample suspected of containing a polynucleotide of the present disclosure, such as a plant cell lysate, particularly a lysate of maize. In some embodiments, a cognate gene of a polynucleotide of the present disclosure or portion thereof can be amplified prior to the step of contacting the nucleic acid sample with a polynucleotide of the present disclosure. The nucleic acid sample is contacted with the polynucleotide to form a hybridization complex. The polynucleotide hybridizes under stringent conditions to a gene encoding a polypeptide of the present disclosure. Formation of the hybridization complex is used to detect a gene encoding a polypeptide of the present disclosure in the nucleic acid sample. Those of skill will appreciate that an isolated nucleic acid comprising a polynucleotide of the present disclosure should lack cross-hybridizing sequences in common with non-target genes that would yield a false positive result. Detection of the hybridization complex can be achieved using any number of well known methods. For example, the nucleic acid sample, or a portion thereof, may be assayed by hybridization formats including but not limited to, solution phase, solid phase, mixed phase or in situ hybridization assays.

Detectable labels suitable for use in the present disclosure include any composition detectable by spectroscopic, radioisotopic, photochemical, biochemical, immunochemical, electrical, optical or chemical means. Useful labels in the present disclosure include biotin for staining with labeled streptavidin conjugate, magnetic beads, fluorescent dyes, radiolabels, enzymes and colorimetric labels. Other labels include ligands which bind to antibodies labeled with fluorophores, chemiluminescent agents and enzymes. Labeling the nucleic acids of the present disclosure is readily achieved such as by the use of labeled PCR primers.

Although the present disclosure has been described in some detail by way of illustration and example for purposes of clarity of understanding, it will be obvious that certain changes and modifications may be practiced within the scope of the appended claims.

### EXAMPLES

### Example 1

### Cloning of Hydrolase, Esterases and the Golgi Targeting Sequences

The following organisms were obtained from the ATCC germplasm resource (found on world wide web at atcc.org). Culture media were prepared using wheat bran as a sole carbohydrate source. Wheat bran (10 g) in 1 L distilled water was autoclaved and the cultures were grown at room temperature for 48 hrs on a bench-top shaker. Total mRNA was isolated using Qiagen's RNA isolation kit and the individual genes were cloned by RT-PCR (sequence listing for primers identified in Table 1). Cloned genes were ligated into pENTR D-TOPO vector (Invitrogen) and sequenced. Confirmed clones were used in the Gateway cloning (Invitrogen) system for making expression vectors.

At ManII (NM121499); Arabidopsis thaliana alpha-mannosidase II is a Golgi localized enzyme responsible for the formation of complex N-glycans in plants. Signal peptide sequence of 207 nucleotides was used to target candidate genes to the cis-Golgi compartment (Saint-Jore-Dupas, et al., (2004); Saint-Jore-Dupas, et al., (2006) Plant Cell 18:3182-3200).

At XYLT (AF272852); Arabidopsis thaliana alpha-1,2-xylosyltransferase is a Golgi localized plant glycosyltransferase that is responsible for catalyzing the transfer of a xylosyl residue to the C2 position of mannose. Targeting sequence of 102 nucleotides was used to localize candidate genes to the medial Golgi compartment (Saint-Jore-Dupas, et al., (2004); Saint-Jore-Dupas, et al., (2006)).

Alpha-2,6-ST (AAA41196.1); Rat alpha-2,6-sialyltransferase is a glycosyltransferase that functions in the Golgi apparatus. Signal peptide sequence of 81 nucleotides was used to localize candidate genes to the trans-Golgi compartment (Wee, et al., (1998) Plant Cell 10:1759-1768; Saint-Jore-Dupas, et al., (2006)).

Acetyl xylan esterases (AXE) hydrolyze ester linkage of the acetyl residues from xylan, which is a constituent of the plant cell wall. The following three genes were targeted to the Golgi apparatus using the various aforementioned signals.
Aspergillus oryzae acetyl xylan esterase (AB167976)
Clostridium thermocellum ATCC 27405 acetyl xylan esterase (YP_{—}001039452)
Neurospora crassa acetyl xylan esterase (XM954034).

Feruloyl esterases hydrolyze feruloyl esters that occur on the arabinosyl residues of GAX. The following three genes were targeted to the Golgi apparatus using various targeting signals.
Aspergillus niger feruloyl esterase A (Y09330)
Clostridium thermocellum xylanase Z (xynZ) gene (YP_{—}001038374)
Neurospora crassa feruloyl esterase B (AJ293029)

Alpha-L-arabinofuranosidase is involved in the hydrolysis of L-arabinosyl linkage from the cell wall. The following three genes were targeted to the Golgi apparatus using various targeting signals.
Clostridium thermocellum ATCC 27405 alpha-L-arabinofuranosidase (NC_{—}009012)
Bacillus subtilis endo-alpha-1,5-L-arabinanase (EU373814)
Aspergillus oryzae alpha-L-arabinofuranosidase B (AB073861)

### Example 2

### Vector Construction and Transformation in Arabidopsis and Maize

Multisite Gateway (Invitrogen) technology was used to generate plant expression vectors. The coding sequence of the above mentioned genes was amplified by PCR and cloned in the entry vector, pENTR (Invitrogen's pENTR.D.TOPO kit). To generate an expression vector driven by a 35S, Ubi or S2A promoter, the LR clonase (Invitrogen) reaction was performed with the gene combinations as is shown in Table 2. The final expression vector contained herbicide and fluorescent marker for transgenic seed sorting. The resulting expression vector was quality checked by restriction digestion mapping and transferred into Agrobacterium tumefaciens LB4404JT by electroporation. The co-integrated DNA from transformed Agrobacterium was transferred in E. coli DH10B and the plasmid DNA from this strain was used to check quality by restriction digestion. These overexpression vectors were transformed into Arabidopsis thaliana ecotype Columbia-0 by Agobacterium-mediated ‘floral-dip’ method (Clough and Bent, (1998) Plant J 16:735-743)

T₀ seeds were grown in the soil and transformants selected based on herbicide resistance and confirmed by PCR-genotyping. RT-PCR was conducted on the transgenic plants to detect the expression of the transgene. Actin was used as a control, both for gene expression as well as for detecting the presence, if any, of the genomic DNA in the in the RNA preparations. Events expressing the transgene were advanced for further characterization. Transgenic plants were analyzed for cell wall acetate content and sugar composition. Eighteen constructs that caused a change in wall composition in Arabidopsis were transformed into maize.

### Example 3

### Localization of Green Fluorescent Protein (GFP) Fused to Golgi Retention Signals on the N-Terminus in the Golgi Apparatus of Arabidopsis

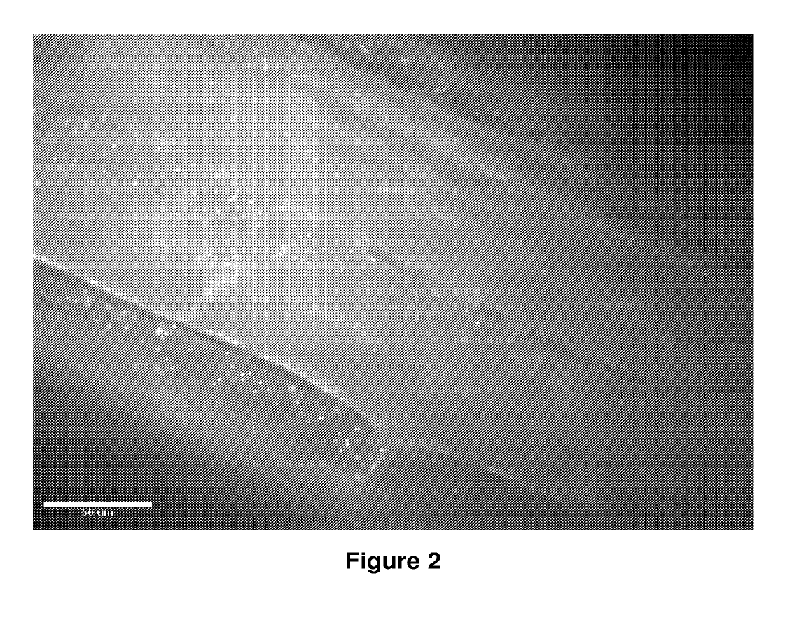

Transgenic plants were selected based on resistance to the selectable maker herbicide, maize-optimized phosphinothricine acetyltransferase (MOPAT). The presence of the transgene was confirmed by genotyping and the expression was studied by RT-PCR. Localization of the Golgi retention signal (see, Example 1 for details) fused to GFP was monitored using a confocal microscope (FIG. 2). Green fluorescence was localized in the disc-shaped, particulate bodies, which, along with the previously available information using these targeting signals, limits it to the Golgi bodies (Saint-Jore-Dupas, et al., (2004)).

### Example 4

### Extraction of Acetate from the Plant Cell Walls and its Determination Using a High Throughput, Coupled Enzyme-Based Biochemical Assay

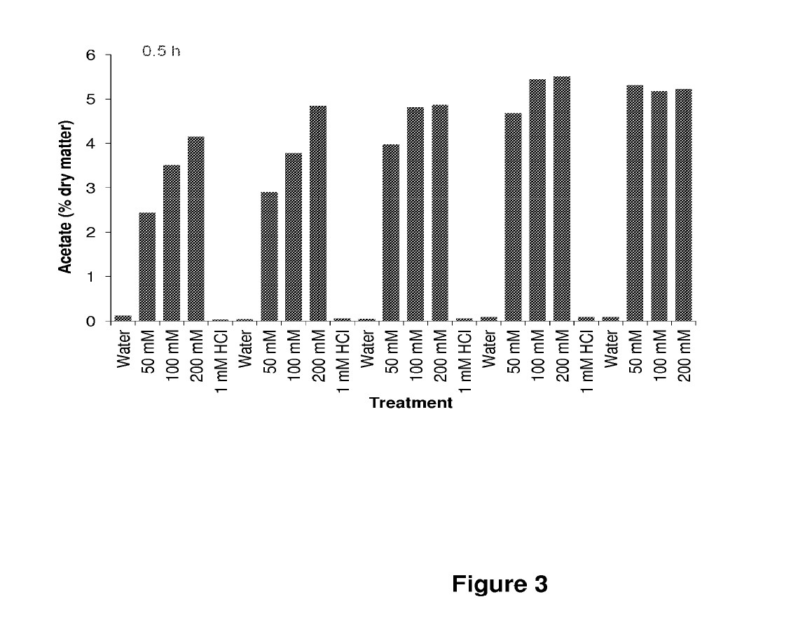

Extraction of acetate from the cell wall
1. Dried plant material was powdered in GenoGrinder using polycarbonate vials (MedPlast Monticello #165699) and steel bead (⅜ inch) for two 30 sec bursts of 650 strokes/min.
2. Various treatments (acidic, neutral and basic) for different time period were used to determine the optimal condition to release acetate from cell wall (FIG. 3). Digestion of cell wall at a concentration of 100 mM NaoH for 4 hrs on inclined shaker at room temperature was selected to be the optimal condition to release acetate from cell wall (FIG. 3).
   Roche acetate assay kit was employed in a modified assay as described below.
Measured 20 mg of corn stalk powder into 1.5 ml microfuge tube (or 1.2 ml micro-titer tube for 96 well format).
Added 100 mM NaOH (750 ul) at room temperature and mixed by continuous shaking for 4 h.
Added 100 ul of 1 M, untitrated HEPES buffer and 50 ul of 1M Tris (pH 8). The final pH of the solution was 7-7.5.
Centrifuged at 14,000×g in a microfuge for 5 min. Removed 300 ul of the supernatant in a fresh tube or microtiter plate (ascertaining that no tissue debris accompanied the solution).
Made 10-fold dilutions of the above supernatants in separate tubes/microtiter plates.
A modified assay using R-Biopharm acetic acid kit (Roche Cat #10148261035) was used as described below to measure acetate in the supernatant.
   Dissolved the contents of bottle 2 in 7 ml and bottle 4 in 1 ml of distilled water in ice.
   Prepared the reaction mixture in ice. (Kept bottle 1 at room temp for 10-15 min before starting the reaction).

| | | |
|---|---|---|
| Bottle 1 (triethanolamine buffer, L-malic acid, MgCl)1 | 1 | ml |
| Bottle 2 (ATP, CoA, NAD) | 0.2 | ml |
| Water | 2.0 | ml |
| Bottle 3 (L-malate dehydrogenase, citrate synthase) | 0.01 | ml |
| Bottle 4 (lyophilizate acetyl-CoA synthetase) | 0.02 | ml |

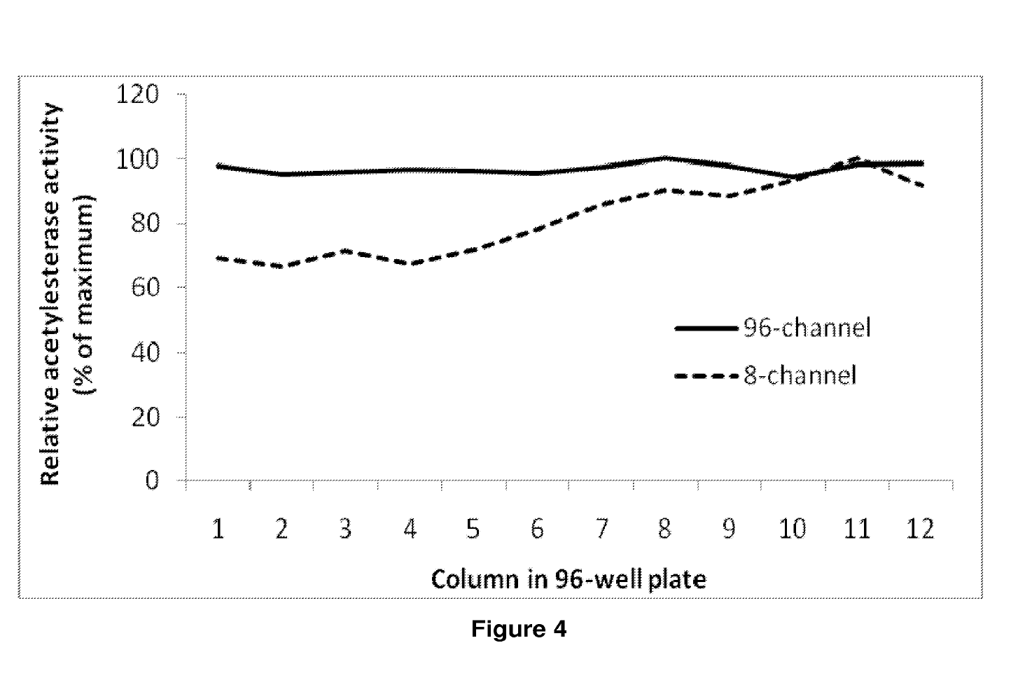

Standards of acetic acid over a range of 0 to 2.5 mM were included in the assay.
Blank reading was made for 160 ul of reaction mixture in flat-base microtiter plate at 340 nm wavelength for one minute. Reaction was started by adding 40 ul of substrate (10-fold diluted cell wall supernatant or standard acetic acid for standards) in 160 ul of reaction mixture and reaction rate was determined over a period of 10 minutes with taking reading after every 10 seconds.
The use of 96 well pipetor was very critical for obtaining consistant results in a highthrough put 96 well format. As shown in FIG. 4, using a standard concentration of acetate (0.36 mM) with two different administration techniques showed a clear gradient difference in 8-channel pipetor as compared to 96 well pipetor an indicator of a difference in reaction rates in various columns as compared to 96 well pipetor where the reaction was initiated in all the wells at the same time.

### Example 5

### Analysis of the Transgenes in Arabidopsis and Maize Expressing Acetylxylanesterase

The amount of acetate in mature dried plant cell wall (stalk tissue) was quantified by the coupled enzyme-based assay described in Example 4. Plants with fungal (Aspergillus oryzae) esterase (AXE), abbreviated as AoAXE, targeted to Golgi compartment showed a significant reduction in wall acetate (up to 40%) without any visible phenotype (Table 3). Note—ND means no change was detected.

TABLE 3

| | Enzyme/ | Man-II | | XylT | | None | |
|---|---|---|---|---|---|---|---|
| Organism | Protein | 35S | S2A | 35S | S2A | 35S | S2A |
| Aspergillus | Acetyl | 40% | ND | 30% | ND | ND | ND |
| oryzae | esterase | | | | | | |
| Neurospora | Acetyl | ND | ND | ND | ND | ND | ND |
| crassa | esterase | | | | | | |
| Clostridium | Acetyl | 25% | 80% | 30% | 70% | ND | ND |
| thermocellum | esterase | | | | | | |

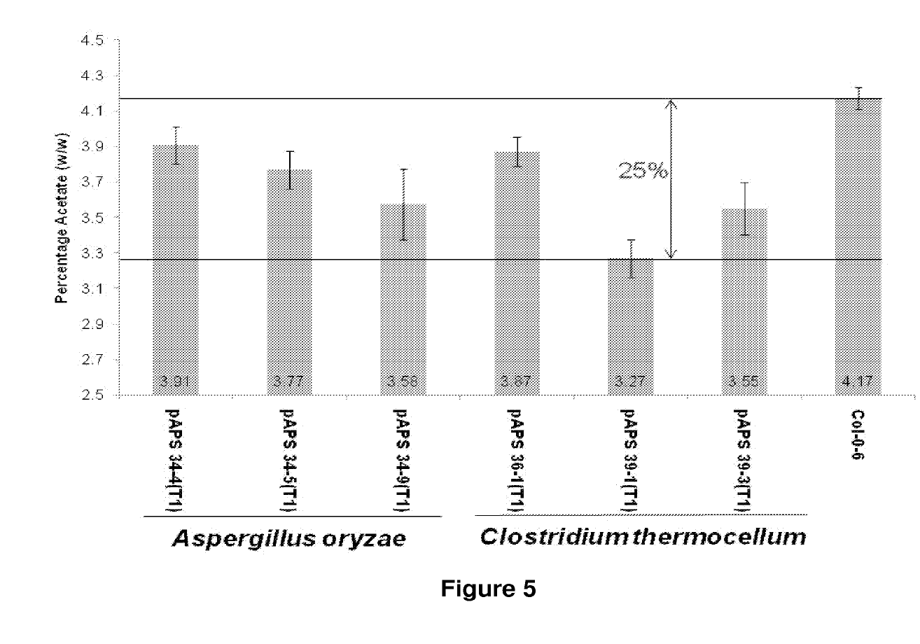

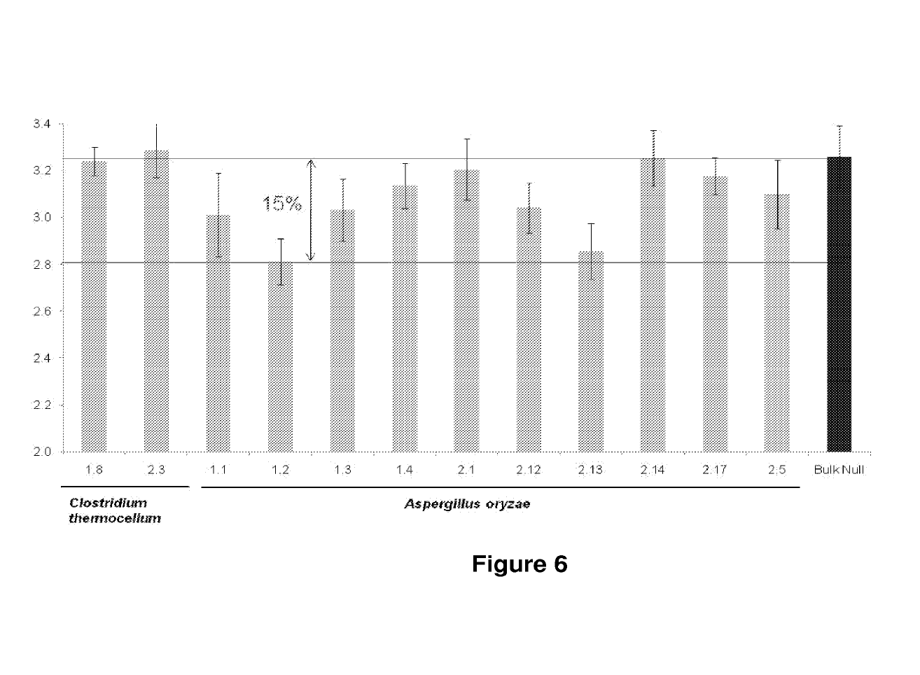

The bacterial (Clostridium thermocellum) esterase (CtAXE) when expressed preferentially in the vascular bundles resulted in up to 80% reduction in acetate, however, the plants did not survived to produce T₁ seed and also exhibited drought symptoms, which is hypothesized to happen because of impaired vascular bundles (Table 3) In T₁ generation from aforementioned Arabidopsis populations, up to 25% reduction in wall acetate was determined by expressing AoAXE and CtAXE in the Golgi apparatus under the control of 35S promoter (FIG. 5). Similarly in maize, over-expressing AoAXE in Golgi under the control of S2A promoter resulted in stable reduction of wall acetate of up to 15%, whereas by over-expressing and CtAXE there was no significant reduction in the wall acetate content (FIG. 6).

Apoplast targeting, as judged from the plants transformed with constructs without a Golgi-targeting signal, did not cause any reduction in acetate content. This shows that Golgi-targeting of this class of enzymes is a must to reduce the acetate content of the cell wall.

### Example 6

### Analysis of Transgenic in Arabidopsis and Maize Expressing Arabinosidase

In Arabidopsis overexpressing fungal and bacterial arabinosidase targeted to the Golgi compartment showed up to 50% reduction in cell wall arabinose content in T₀ plants without any visible phenotype (Table 4). Note—ND means no change was detected.

TABLE 4

| | Enzyme/ | Man-II | | XylT | | SialT | | None | |
|---|---|---|---|---|---|---|---|---|---|
| Organism | Protein | 35S | S2A | 35S | S2A | 35S | S2A | 35S | S2A |
| Aspergillus | Arabinosidase | 40% | ND | 30% | ND | ND | ND | ND | ND |
| oryzae | | | | | | | | | |
| Bacillus Subtilis | Arabinosidase | 50% | ND | 40% | ND | 35% | ND | ND | ND |
| Neurospora | Arabinosidase | ND | ND | ND | ND | 25% | ND | ND | ND |
| crassa | | | | | | | | | |

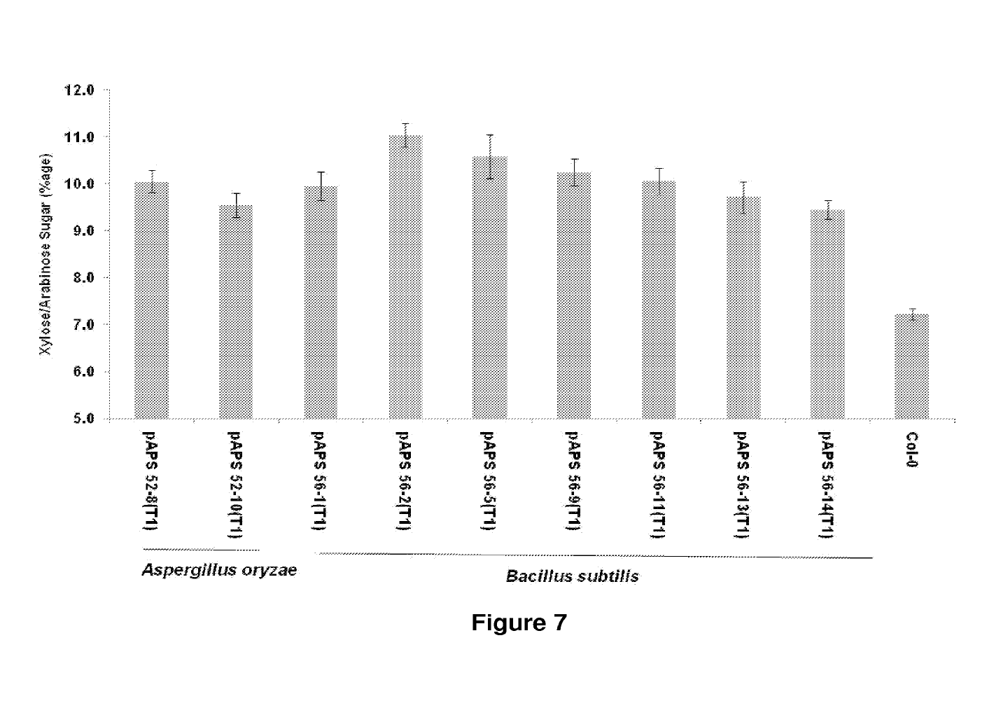

Stable reduction in arabinose content was determined in T₁ plants in Arabidopsis under the control of 35S promoter (FIG. 7). Xylose to arabinose ratio in T₁ events increased in the events derived using Aspergillus niger arabinosidase by up to 35% and in those derived using the Bacillus subtilis enzymes by up to 60% as compared to wildtype. It is likely that these arabinosidases remove arabinosyl residues from pectin, not from glucuronoarabinoxylan. There is little to no arabinose on the glucuronoxylan of Arabidopsis (Oikawa, et al., PLoS One 5:e15481; Pena, et al., (2007).

### Example 7

### Ferulic Acid Determination in Maize Stover Using HPLC

Total cell wall (20 mg) was digested with 2 ml of anaerobic 2M NaOH overnight at room temperature using inclined shaker. The digestion was titrated with 0.36 ml of 6M HCl.
Samples were placed in a refrigerator for 2 h to allow settling of particulate matter and then centrifuged twice at 14000 g for 10 minutes.
Supernatant aliquot was removed from the tubes and stored at 4° C. until analyzed by high pressure liquid chromatography, which was done within 4 d of sample extraction.
Analysis of Ferulic Acid and Coumaric Acid by HPLC—The purpose of this procedure is to analyze aqueous plant digest for ferulic acid and coumaric acid as separated by reversed-phase HPLC and quantified by UV using a PDA detector.

Reagents and Supplies
Ferulic Acid (ICN Biomedicals Inc. Cat. #101685)
p—Coumaric Acid (ICN Biomedicals Inc. Cat. #102576)
Acetonitrile—HPLC Grade (OmniSolv, AX0142-1)
Purified water equivalent to 18 MΩ-cm resistivity
Methanol—HPLC Grade
Trifluoroacetic Acid* (TFA) (J. T. Baker, W729-05)
Volumetric flasks—25 mL, 100 mL, 200 mL
Centrifugal filters, 0.2 μm, 500 ul
Micropipettor tips for P200 and P1000
Autosampler vials with glass inserts

Equipment
Adjustable micropipettors (20 μL, 200 μL and 1000 μL)
Vortex mixer
Analytical balance
Sonic water bath
HPLC pumping system with at least two solvent reservoirs (Waters Alliance 2695)
Waters Alliance 2695 Autosampler or equivalent
Waters Spherisorb® 5 μm ODS2 HPLC analytical column 4.6×250 mm
Waters Photodiode array (PDA) 996 Detector
Chromatography software package (Waters Empower Pro)
Personal Protective Equipment

### Procedure

### Preparing Standards

Stock standards are prepared separately using 50 mg of each compound and diluted with methanol to 25 ml in a volumetric flask for a final concentration of 2.0 mg/ml.
Working standard: Aliquots of the stock standards are combined in volumetric flasks and diluted with purified water to provide adequate standards at final concentrations of 200 μg/ml, 100 μg/ml, 50 μg/ml, 25 μg/ml, 10 μg/ml and 5 μg/ml to be used as an external curve for quantitation.

Sample Preparation
All samples should be uniquely labeled and identified by the customer or lab personnel.
All samples should be analyzed within one week as the compounds appear to degrade over time at extreme pH.
All samples are filtered using a centrifugal filter at 0.2 μm. The filtrate is transferred to a labeled autosampler vial with an insert. A visual inspection is performed and any air bubbles are removed.
If not immediate place on the instrumentation for analysis, samples are stored at ˜5° C.

Mobile Phase Preparation

Eluent A: Purified Water with 0.05% TFA
Make fresh weekly or as needed, degas (5 min.) prior to use.

Eluent B: Acetonitrile with 0.05% TFA
Make fresh weekly or as needed, degas (5 min.) prior to use.

System Preparation
The Waters Alliance system 2695 is recommended or equivalent. Injection volume is 10 μl.

Gradient table for ferulic/coumaric acid analysis:

| Time | Flow | % Eluent A | % Eluent B | Gradient |
|---|---|---|---|---|
| Initial | 0.6 | 75 | 25 | — |
| 5.00 | 0.5 | 75 | 25 | 6 |
| 20.0 | 0.5 | 25 | 75 | 6 |
| 21.0 | 0.6 | 10 | 90 | 6 |
| 25.0 | 0.6 | 10 | 90 | 6 |
| 26.0 | 0.6 | 75 | 25 | 6 |
| 40 | 0 | 75 | 25 | 6 |

Data acquisition ends at 27 min.
PDA Detector settings are as follows:
Wavelength Start at 190
Wavelength End at 800
Quantitation at wavelength 317

Sample Analysis

Samples are calibrated using a six level standard curve. To run samples, inject a 10 μL water blank before and after the calibration curve. The standards are injected immediate proceeding and immediately following the sample set. View the calibration curve and use sample data if R² is at least 0.99 and any check standards are within 10%.

### Example 8

### Analysis of Transgenic Maize Expressing Feruloyl Esterase

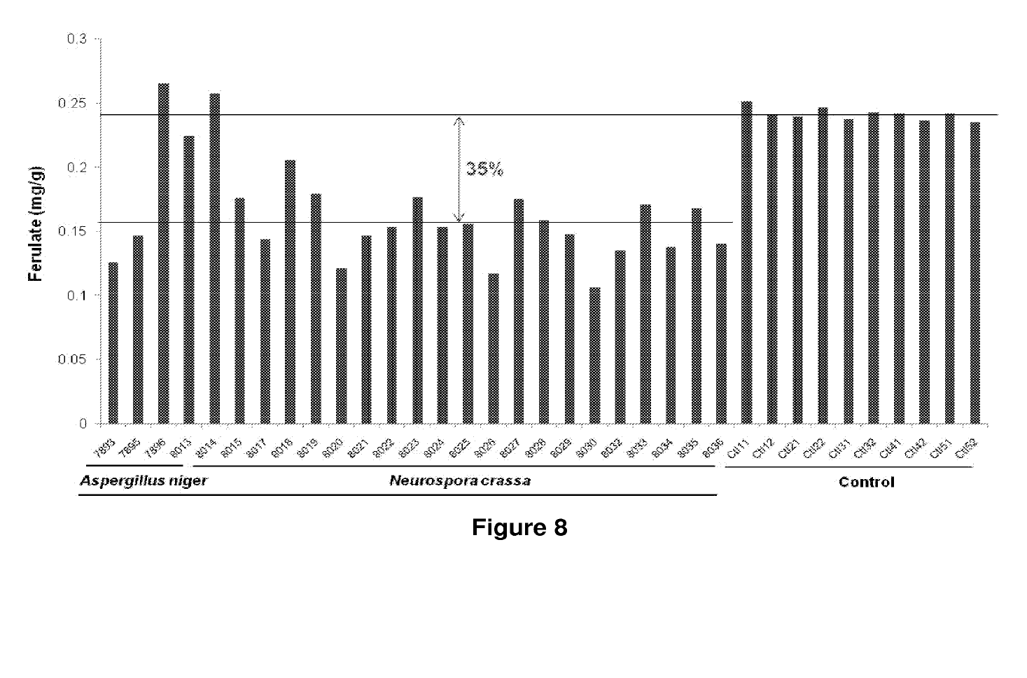

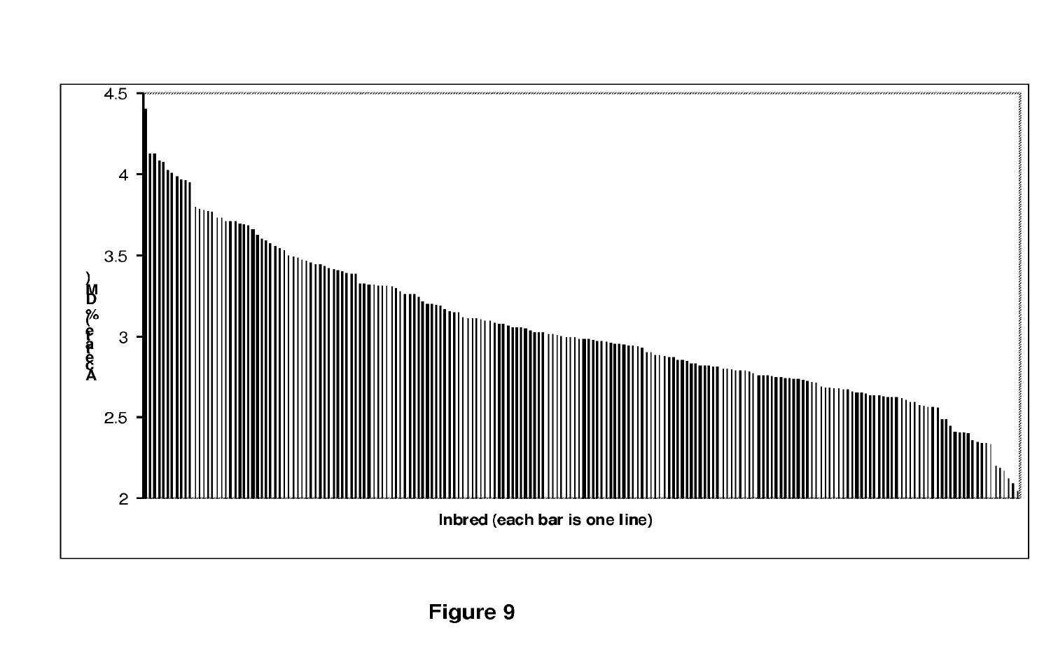

Plants were harvested in the green-house at 100% anthesis stage. Stalks were lyophilized for 10 days. Lower most internode was used for the determination of ferulic acid determination. Stalk samples were ground into fine powder using genogrinder (as discussed in example 4) and ferulic acid was determined as by the Example 7. Significant reduction of up to 35% ferulic acid was determined in T₀ individuals overexpressing Aspergillus niger and Neurospora crassa feruloyl-esterase in Golgi compartment under the control of S2A promoter (FIG. 8).

### Example 9

### Genetic Variation for Cell Wall Acetate Content in Maize Diversity Population

To determine genetic variation in maize diversity population, a set of 220 inbreds were grown in four replications at Puerto Rico. Mature cobs were harvested from four plants in each replication and were pooled together for grinding into approximately 1 mm size particles. Total acetate was determined by the biochemical assay developed in-house as described above in example 4. Two fold variation of wall acetate was determined in myriad diversity population as is shown in FIG. 9.

### Example 10

### Identification of QTL for Wall Acetate Using Association Genetics Approach

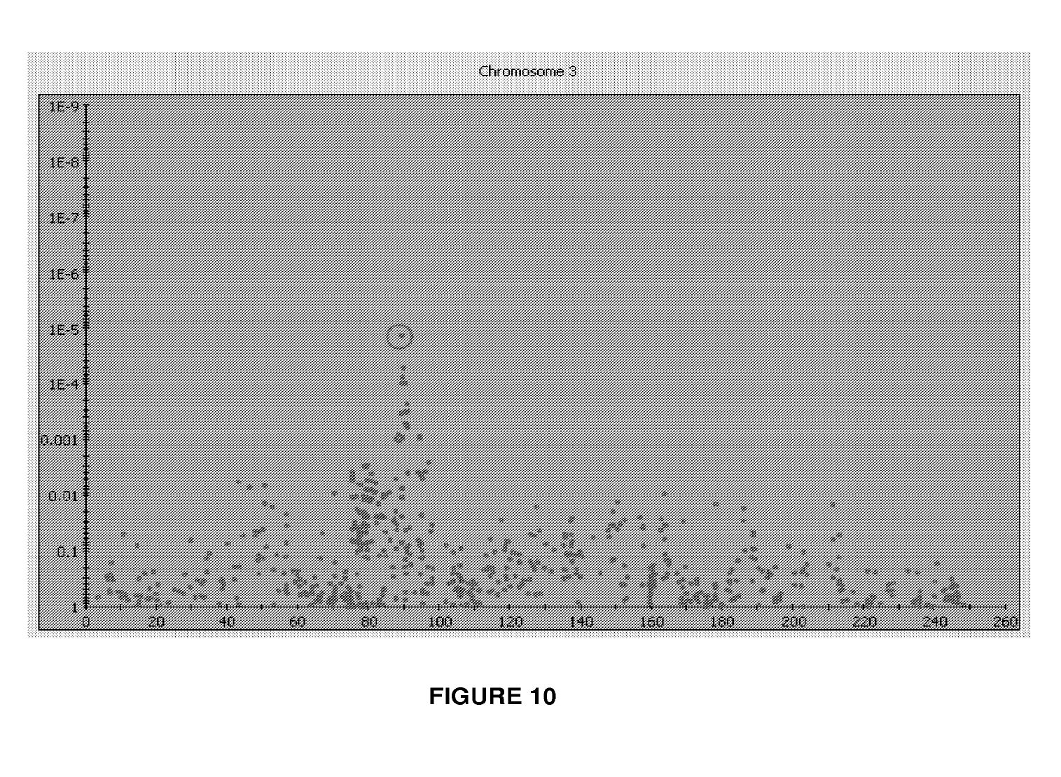

Using the in-house developed tool for association genetics, variation for cell wall acetate was mapped to a strong QTL at chromosome 3 (FIG. 10). Further by using gene-order map tool we identified a gene candidate which was annotated as pectin acetylesterase. The ortholog from Arabidopsis was identified as a annotated gene model At3g09410. Topology prediction shows that it is a type two membrane protein.

### Example 11

### Functional Characterization of Arabidopsis (At3g09410) Ortholog for Pectin Acetylesterase

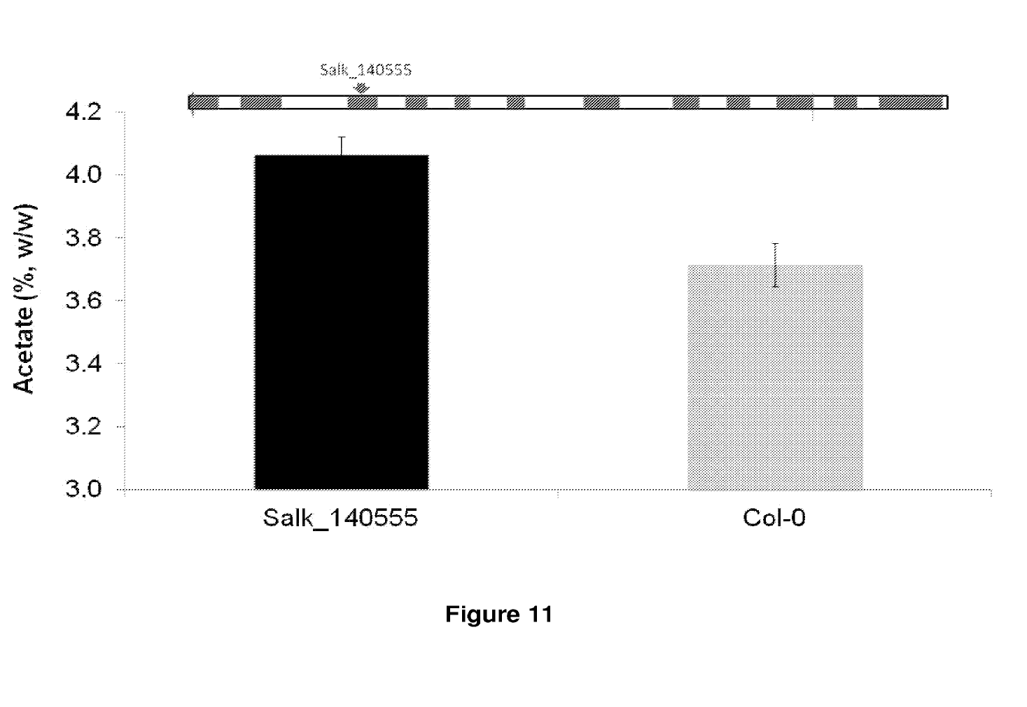

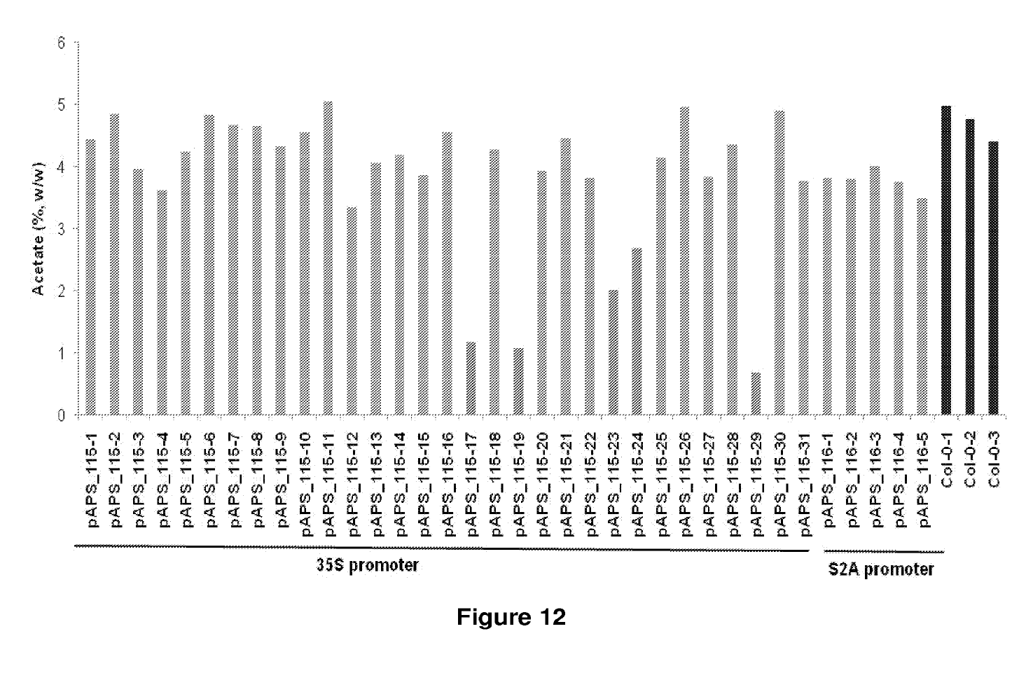

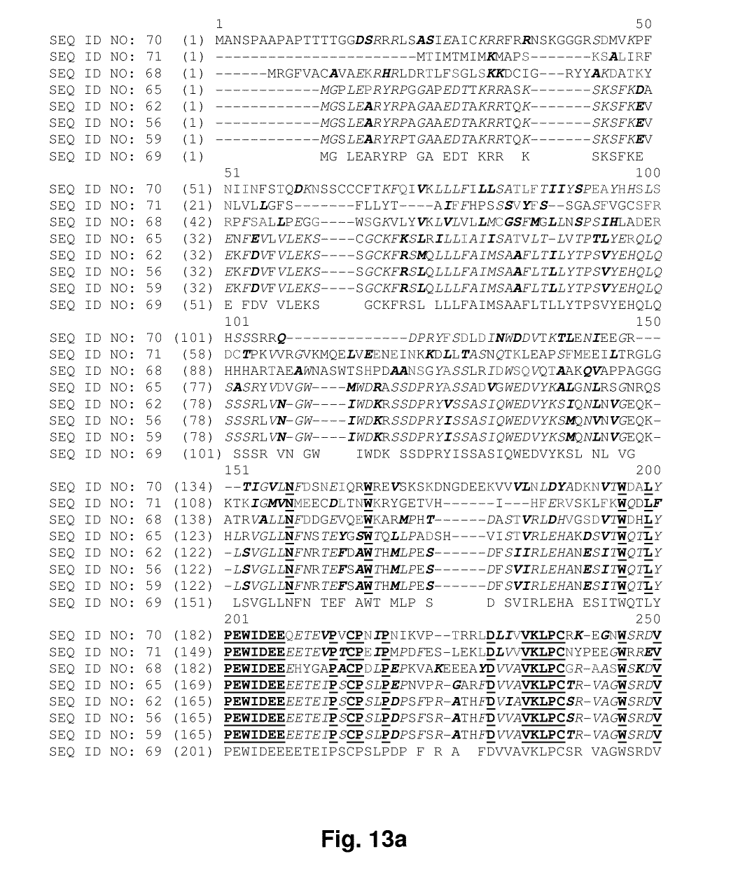

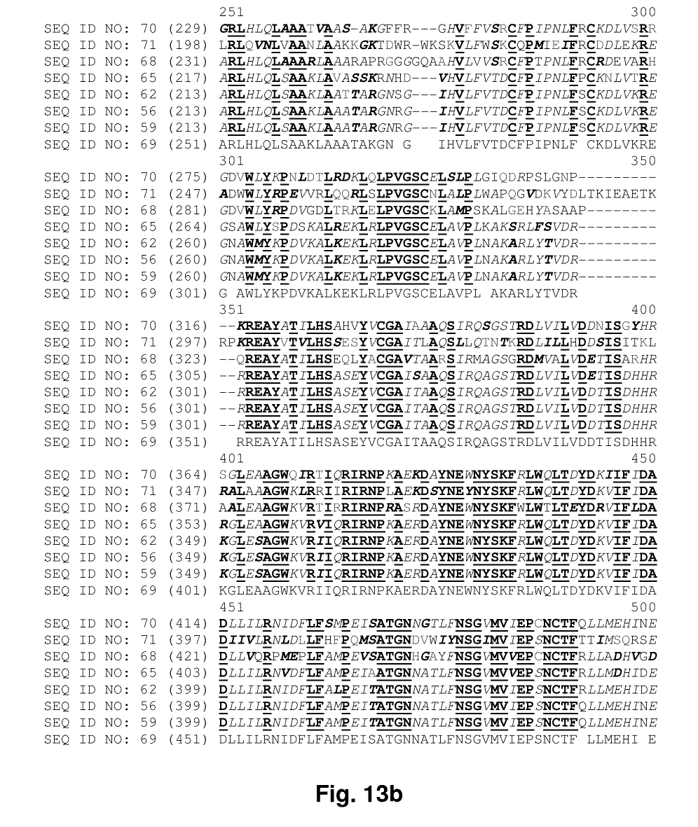

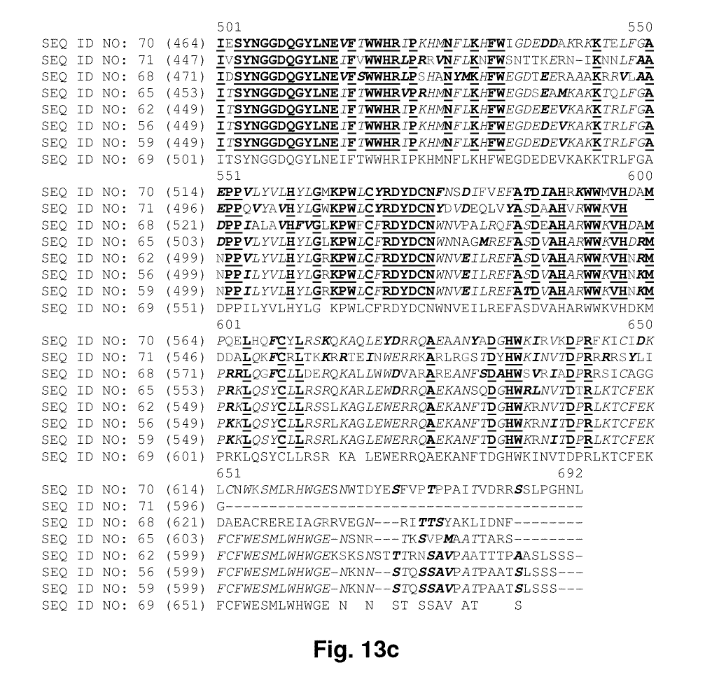

Knock-out mutant from At3g09410 was ordered from Salk collection (found on the world wide web at arabidopsis.org) and was characterized for the acetate content in stem tissue. There was an increase in acetylation (about 10%) in mutant plants as compared to control (FIG. 11). This suggests that the protein is an acetylesterase and by knocking-out the expression of it would increase the accumulation of acetate in the cell wall. Further the overexpression lines for At3g09410 gene in Arabidopsis were generated with 35S and S2A promoter. There was a significant reduction in wall acetylation in over-expression lines (T₀) as is shown in FIG. 12.

### Example 12

### Transformation and Regeneration of Transgenic Plants

Immature maize embryos from greenhouse donor plants are bombarded with a plasmid containing the esterase sequence operably linked to the drought-inducible promoter RAB17 promoter (Vilardell, et al., (1990) Plant Mol Biol 14:423-432) and the selectable marker gene PAT, which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox® bleach plus 0.5% Micro detergent for 20 minutes and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the esterase sequence operably linked to an ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water
10 μl (1 μg) DNA in Tris EDTA buffer (1 μg total DNA)
100 μl 2.5M CaC1₂
10 μl 0.1M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased drought tolerance. Assays to measure improved drought tolerance are routine in the art and include, for example, increased kernel-earring capacity yields under drought conditions when compared to control maize plants under identical environmental conditions. Alternatively, the transformed plants can be monitored for a modulation in meristem development (i.e., a decrease in spikelet formation on the ear). See, for example, Bruce, et al., (2002) Journal of Experimental Botany 53:1-13.

Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite® (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose and 2.0 mg/l 2,4-D (brought to volume with D-I H₂O following adjustment to pH 5.8 with KOH); 3.0 g/l Gelrite® (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose and 1.0 ml/l of 0.1 mM abscisic acid (brought to volume with polished D-I H₂O after adjusting to pH 5.6); 3.0 g/l Gelrite® (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL and 0.40 g/l glycine brought to volume with polished D-I H₂O), 0.1 g/l myo-inositol and 40.0 g/l sucrose (brought to volume with polished D-I H₂O after adjusting pH to 5.6) and 6 g/l Bacto™-agar (added after bringing to volume with polished D-I H₂O), sterilized and cooled to 60° C.

### Example 13

### Agrobacterium-Mediated Transformation

For Agrobacterium-mediated transformation of maize with an antisense sequence of the Zmesterasesequence of the present disclosure, preferably the method of Zhao is employed (U.S. Pat. No. 5,981,840 and PCT Patent Publication WO 1998/32326, the contents of which are hereby incorporated by reference). Briefly, immature embryos are isolated from maize and the embryos contacted with a suspension of Agrobacterium, where the bacteria are capable of transferring the esterase sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants. Plants are monitored and scored for a modulation in meristem development. For instance, alterations of size and appearance of the shoot and floral meristems and/or increased yields of leaves, flowers and/or fruits are monitored.

### Example 14

### Sugarcane Transformation

This protocol describes routine conditions for production of transgenic sugarcane lines. The same conditions are close to optimal for number of transiently expressing cells following bombardment into embryogenic sugarcane callus. See also, Bower, et al., (1996). Molec Breed 2:239-249; Birch and Bower, (1994). Principles of gene transfer using particle bombardment. In Particle Bombardment Technology for Gene Transfer, Yang and Christou, eds (New York: Oxford University Press), pp. 3-37 and Santosa, et al., (2004), Molecular Biotechnology 28:113-119, incorporated herein by reference.

### Sugarcane Transformation Protocol

1. Subculture callus on MSC3, 4 days prior to bombardment:
(a) Use actively growing embryogenic callus (predominantly globular pro-embryoids rather than more advanced stages of differentiation) for bombardment and through the subsequent selection period.
(b) Divide callus into pieces around 5 mm in diameter at the time of subculture and use forceps to make a small crater in the agar surface for each transferred callus piece.
(c) Incubate at 28° C. in the dark, in deep (25 mm) Petri dishes with micropore tape seals for gas exchange.
   2. Place embryogenic callus pieces in a circle (˜2.5 cm diameter), on MSC3Osm medium. Incubate for 4 hours prior to bombardment.
   3. Sterilize 0.7 μm diameter tungsten (Grade M-10, Bio-Rad #165-2266) in absolute ethanol. Vortex the suspension, then pellet the tungsten in a microfuge for ˜30 seconds. Draw off the supernatant and resuspend the particles at the same concentration in sterile H₂O. Repeat the washing step with sterile H₂0 twice and thoroughly resuspend particles before transferring 50 μl aliquots into microfuge tubes.
   4. Add the precipitation mix components:

| Component (stock solution) | Volume to add | Final concin mix | |
|---|---|---|---|
| Tungsten (100 μg/μl in H₂0) | 50 μl | 38.5 | μg/μl |
| DNA (1 μg/μl) | 10 μl | 0.38 | μg/μl |
| CaCl₂ (2.5M in H20) | 50 μl | 963 | mM |
| Spermidine free base (0.1M in H₂0) | 20 μl | 15 | mM |

5. Allow the mixture to stand on ice for 5 min. During this time, complete steps 6-8 below.
6. Disinfect the inside of the ‘gene gun’ target chamber by swabbing with ethanol and allow it to dry.
7. Adjust the outlet pressure at the helium cylinder to the desired bombardment pressure.
8. Adjust the solenoid timer to 0.05 seconds. Pass enough helium to remove air from the supply line (2-3 pulses).
9. After 5 min on ice, remove (and discard) 100 μl of supernatant from the settled precipitation mix.
10. Thoroughly disperse the particles in the remaining solution.
11. Immediately place 4 μl of the dispersed tungsten-DNA preparation in the center of the support screen in a 13 mm plastic syringe filter holder.
12. Attach the filter holder to the helium outlet in the target chamber.
13. Replace the lid over the target tissue with a sterile protective screen. Place the sample into the target chamber, centered 16.5 cm under the particle source and close the door.
14. Open the valve to the vacuum source. When chamber vacuum reaches 28″ of mercury, press the button to apply the accelerating gas pulse, which discharges the particles into the target chamber.
15. Close the valve to the vacuum source. Allow air to return slowly into the target chamber through a sterilizing filter. Open the door, cover the sample with a sterile lid and remove the sample dish from the chamber.
16. Repeat steps 10-15 for consecutive target plates using the same precipitation mix, filter and screen.
17. Approximately 4 hours after bombardment, transfer the callus pieces from MSC3Osm to MSC3.
18. Two days after shooting, transfer the callus onto selection medium. During this transfer, divide the callus into pieces ˜5 mm in diameter, with each piece being kept separate throughout the selection process.
19. Subculture callus pieces at 2-3 week intervals.
20. When callus pieces grow to ˜5 to 10 mm in diameter (typically 8 to 12 weeks after bombardment) transfer onto regeneration medium at 28° C. in the light.
21. When regenerated shoots are 30-60 mm high with several well-developed roots, transfer them into potting mix with the usual precautions against mechanical damage, pathogen attack and desiccation until plantlets are established in the greenhouse.

### Example 15

### Soybean Embryo Transformation

Soybean embryos are bombarded with a plasmid containing a esterase sequence operably linked to an ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette comprising a esterase sense sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 16

### Sunflower Meristem Tissue Transformation

Sunflower meristem tissues are transformed with an expression cassette containing a esterase sequence operably linked to a ubiquitin promoter as follows (see also, EP Patent Number 0 486233, herein incorporated by reference and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox® bleach solution with the addition of two drops of Tween® 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant., 15:473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6 and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the esterase gene operably linked to the ubiquitin promoter is introduced into Agrobacterium strain EHA105 via freeze-thawing as described by Holsters, et al., (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bacto® peptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for a modulation in meristem development (i.e., an alteration of size and appearance of shoot and floral meristems).

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% Gelrite®, pH 5.6) and grown under conditions described for explant culture. The upper portion of the seedling is removed, a 1 cm vertical slice is made in the hypocotyl and the transformed shoot inserted into the cut. The entire area is wrapped with Parafilm® to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by esterase activity analysis of leaf extracts while transgenic seeds harvested from NPTII-positive T₀ plants are identified by esterase activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox® bleach solution with the addition of two to three drops of Tween® 20 per 100 ml of solution, then rinsed three times with distilled water. Sterilized seeds are imbibed in the dark at 26° C. for 20 hours on filter paper moistened with water. The cotyledons and root radical are removed and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar) and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bacto® peptone and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD₆₀₀. Particle-bombarded explants are transferred to GBA medium (374E) and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for a modulation in meristem development (i.e., an alteration of size and appearance of shoot and floral meristems). After positive (i.e., a change in esterase expression) explants are identified, those shoots that fail to exhibit an alteration in esterase activity are discarded and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for altered esterase expression are grafted to Pioneer hybrid 6440 in vitro-grown sunflower seedling rootstock. The rootstocks are prepared in the following manner. Seeds are dehulled and surface-sterilized for 20 minutes in a 20% Clorox® bleach solution with the addition of two to three drops of Tween® 20 per 100 ml of solution, and are rinsed three times with distilled water. The sterilized seeds are germinated on the filter moistened with water for three days, then they are transferred into 48 medium (half-strength MS salt, 0.5% sucrose, 0.3% Gelrite® pH 5.0) and grown at 26° C. under the dark for three days, then incubated at 16-hour-day culture conditions. The upper portion of selected seedling is removed, a vertical slice is made in each hypocotyl and a transformed shoot is inserted into a V-cut. The cut area is wrapped with Parafilm®. After one week of culture on the medium, grafted plants are transferred to soil. In the first two weeks, they are maintained under high humidity conditions to acclimatize to a greenhouse environment.

### Example 17

### Agrobacterium Mediated Grass Transformation

Grass plants may be transformed by following the Agrobacterium mediated transformation of Luo, et al., (2004) Plant Cell Rep (2004) 22:645-652.

### Materials and Methods

### Plant Material

A commercial cultivar of creeping bentgrass (Agrostis stolonifera L. cv. Penn-A-4) supplied by Turf-Seed (Hubbard, Ore.) can be used. Seeds are stored at 4° C. until used.

### Bacterial Strains and Plasmids

Agrobacterium strains containing one of 3 vectors are used. One vector includes a pUbi-gus/Act1-hyg construct consisting of the maize ubiquitin (ubi) promoter driving an intron-containing b-glucuronidase (GUS) reporter gene and the rice actin 1 promoter driving a hygromycin (hyg) resistance gene. The other two pTAP-arts/35S-bar and pTAP-barnase/Ubi-bar constructs are vectors containing a rice tapetum-specific promoter driving either a rice tapetum-specific antisense gene, rts (Lee, et al., (1996) Int Rice Res Newsl 21:2-3) or a ribonuclease gene, barnase (Hartley, (1988) J Mol Biol 202:913-915), linked to the cauliflower mosaic virus 35S promoter (CaMV 35S) or the rice ubi promoter (Huq, et al., (1997) Plant Physiol 113:305) driving the bar gene for herbicide resistance as the selectable marker.

### Induction of Embryogenic Callus and Agrobaterium-Mediated Transformation

Mature seeds are dehusked with sand paper and surface sterilized in 10% (v/v) Clorox® bleach (6% sodium hypochlorite) plus 0.2% (v/v) Tween® 20 (Polysorbate 20) with vigorous shaking for 90 min. Following rinsing five times in sterile distilled water, the seeds are placed onto callus-induction medium containing MS basal salts and vitamins (Murashige and Skoog, (1962) Physiol Plant 15:473-497), 30 g/l sucrose, 500 mg/l casein hydrolysate, 6.6 mg/l 3,6-dichloro-o-anisic acid (dicamba), 0.5 mg/l 6-benzylaminopurine (BAP) and 2 g/l Phytagel. The pH of the medium is adjusted to 5.7 before autoclaving at 120° C. for 20 min. The culture plates containing prepared seed explants are kept in the dark at room temperature for 6 weeks. Embryogenic calli are visually selected and subcultured on fresh callus-induction medium in the dark at room temperature for 1 week before co-cultivation.

### Transformation

The transformation process is divided into five sequential steps: agro-infection, co-cultivation, antibiotic treatment, selection and plant regeneration. One day prior to agro-infection, the embryogenic callus is divided into 1- to 2-mm pieces and placed on callus-induction medium containing 100 μM acetosyringone. A 10-ml aliquot of Agrobacterium suspension (OD=1.0 at 660 nm) is then applied to each piece of callus, followed by 3 days of co-cultivation in the dark at 25° C. For the antibiotic treatment step, the callus is then transferred and cultured for 2 weeks on callus-induction medium plus 125 mg/l cefotaxime and 250 mg/l carbenicillin to suppress bacterial growth. Subsequently, for selection, the callus is moved to callus-induction medium containing 250 mg/1 cefotaxime and 10 mg/l phosphinothricin (PPT) or 200 mg/l hygromycin for 8 weeks. Antibiotic treatment and the entire selection process is performed at room temperature in the dark. The subculture interval during selection is typically 3 weeks. For plant regeneration, the PPT- or hygromycin-resistant proliferating callus is first moved to regeneration medium (MS basal medium, 30 g/l sucrose, 100 mg/l myo-inositol, 1 mg/l BAP and 2 g/l Phytagel) supplemented with cefotaxime, PPT or hygromycin. These calli are kept in the dark at room temperature for 1 week and then moved into the light for 2-3 weeks to develop shoots. Small shoots are then separated and transferred to hormone-free regeneration medium containing PPT or hygromycin and cefotaxime to promote root growth while maintaining selection pressure and suppressing any remaining Agrobacterium cells. Plantlets with well-developed roots (3-5 weeks) are then transferred to soil and grown either in the greenhouse or in the field.

### Staining for GUS Activity

GUS activity in transformed callus is assayed by histochemical staining with 1 mM 5-bromo-4-chloro-3-indolyl-b-d-glucuronic acid (X-Gluc, Biosynth, Staad, Switzerland) as described in Jefferson, (1987) Plant Mol Biol Rep 5:387-405. The hygromycin-resistant callus surviving from selection was incubated at 37 C overnight in 100 μl of reaction buffer containing X-Gluc. GUS expression is then documented by photography.

### Vernalization and Out-Crossing of Transgenic Plants

Transgenic plants are maintained out of doors in a containment nursery (3-6 months) until the winter solstice in December. The vernalized plants are then transferred to the greenhouse and kept at 25° C. under a 16/8 h [day/light (artificial light)] photoperiod and surrounded by non-transgenic wild-type plants that physically isolated them from other pollen sources. The plants will initiate flowering 3-4 weeks after being moved back into the greenhouse. They are out-crossed with the pollen from the surrounding wild-type plants. The seeds collected from each individual transgenic plant are germinated in soil at 25° C. and T1 plants are grown in the greenhouse for further analysis.

### Seed Testing

Test of the Transgenic Plants and their Progeny for Resistance to PPT

Transgenic plants and their progeny are evaluated for tolerance to glufosinate (PPT) indicating functional expression of the bar gene. The seedlings are sprayed twice at concentrations of 1-10% (v/v) Finale© (AgrEvo USA, Montvale, N.J.) containing 11% glufosinate as the active ingredient. Resistant and sensitive seedlings are clearly distinguishable 1 week after the application of Finale© in all the sprayings.

### Statistical Analysis

Transformation efficiency for a given experiment is estimated by the number of PPT-resistant events recovered per 100 embryogenic calli infected and regeneration efficiency is determined using the number of regenerated events per 100 events attempted. The mean transformation and regeneration efficiencies are determined based on the data obtained from multiple independent experiments. A Chi-square test can be used to determine whether the segregation ratios observed among T1 progeny for the inheritance of the bar gene as a single locus fit the expected 1:1 ratio when out-crossed with pollen from untransformed wild-type plants.

### DNA Extraction and Analysis

Genomic DNA is extracted from approximately 0.5-2 g of fresh leaves essentially as described by Luo, et al., (1995) Mol Breed 1:51-63. Ten micrograms of DNA is digested with HindIII or BamHI according to the supplier's instructions (New England Biolabs, Beverly, Mass.). Fragments are size-separated through a 1.0% (w/v) agarose gel and blotted onto a Hybond-N+ membrane (Amersham Biosciences, Piscataway, N.J.). The bar gene, isolated by restriction digestion from pTAP-arts/35S-bar, is used as a probe for Southern blot analysis. The DNA fragment is radiolabeled using a Random Priming Labeling kit (Amersham Biosciences) and the Southern blots are processed as described by Sambrook, et al., (1989) Molecular cloning: a laboratory manual, 2nd edn. Cold Spring Harbor Laboratory Press, New York.

### Polymerase Chain Reaction

The two primers designed to amplify the bar gene are as follows: 5′-GTCTGCACCATCGTCAACC-3′ (SEQ ID NO: 52), corresponding to the proximity of the 5′ end of the bar gene and 5′-GAAGTCCAGCTGCCAGAAACC-3′ (SEQ ID NO: 53), corresponding to the 3′ end of the bar coding region. The amplification of the bar gene using this pair of primers should result in a product of 0.44 kb. The reaction mixtures (25 μl total volume) consist of 50 mM KCl, 10 mM Tris-HCl (pH 8.8), 1.5 mM MgCl2, 0.1% (w/v) Triton X-100, 200 μM each of dATP, dCTP, dGTP and dTTP, 0.5 μM of each primer, 0.2 μg of template DNA and 1 U Taq DNA polymerase (QIAGEN, Valencia, Calif.). Amplification is performed in a Stratagene Robocycler Gradient 96 thermal cycler (La Jolla, Calif.) programmed for 25 cycles of 1 min at 94° C. (denaturation), 2 min at 55′C (hybridization), 3 min at 72° C. (elongation) and a final elongation step at 72° C. for 10 min. PCR products are separated on a 1.5% (w/v) agarose gel and detected by staining with ethidium bromide.

### Example 18

### Expression of Multiple Enzymes Proteins Fused Together in Transgenic Plants

One desirable method to express multiple enzymes or proteins together, particularly at the same intracellular site, is to fuse them together. This is advantageous in that the fusion protein containing multiple enzymes will segregate as a single locus, facilitating the combining of even more genes as well as improving the outcome of the fused enzymes in cases where, in particular, metabolic channeling is involved. The transcription cassette encoding these fusion proteins can be driven by a single promoter (e.g. S2A, UBI, 35S etc.). In general, a 15 amino spacer/linker (3× GGGGS or glycine-glycine-glycine-glycine-serine) is inserted inbetween the two proteins to facilitate the proper folding and thus function of these proteins. The residues like glycine and serine are used so that the adjacent protein domains have the degree of freedom to move relative to one another. In some cases, LINKER, computer software is also used to select the sequence of spacer/linker (Crasto and Feng, (2000) Protein Eng (2000 May) 13(5):309-12. pmid:10835103). In a separate set of similar vectors, an epitope tag, such as HA or FLAG is also added in N or C terminals to detect fusion proteins in a transgenic plant by immuno-detection using anti-epitope antibodies. The final expression vector contains herbicide and fluorescent marker for transgenic seed sorting. The resulting expression vector is analyzed by restriction digestion mapping to ensure quality control and transferred into Agrobacterium tumefaciens LB4404JT by electroporation. The co-integrated DNA from transformed Agrobacterium is transferred in E. Coli DH10B and the plasmid DNA from this strain was used to determine its quality by restriction digestion. These over-expression vectors are transformed into Arabidopsis thaliana ecotype Columbia-0 by Agobacterium-mediated ‘Floral-Dip’ method (Clough and Bent, (1998) Plant Journal 16:735). Transgenic events are generated containing expression vectors for these fusion proteins. T₀ seeds are screened for T₁ transformants in soil for herbicide resistance. The transgenic plants are characterized at molecular level for the presence of transgenes in the genome and mRNA expression by genomic PCR and RT-PCR analyses, respectively. The plants expressing multiple genes as expected were further examined for morphological and biochemical phenotypes such as acetate and ferulate contents of the wall. The enzymes acetylesterase, feruloylesterase, arabinosidase and glucuronosidase from various organisms are fused in different double combinations and a triple combination. As these are all Type-II membrane proteins, the transmembrane domains (TMD) of all the enzymes but one are removed by molecular means in the fusion proteins. A TMD near the N-terminus of each of these enzymes retains these enzymes in the Golgi apparatus. Type-II enzymes are known to be functional with a deleted TMD as shown in Edwards, et al., (1999) Plant Journal 19:691-697.

### Example 19

### Alternative Methods of Reducing Acetate and/or Ferulate Content in Plant Biomass

In addition to methods of reducing the acetate and/or ferulate content in plant biomass for example, by expressing acetyl and/or feruloyl esterases as disclosed herein, methods to reduce the formation of acetate and/or ferulate are also contemplated. For example, suppressing the expression or the activity of an enzyme or enzymes involved in the formation of acetate and/or ferulate result in reduced acetate and/or ferulate content in the plant. In an embodiment, an acetyl transferase and/or a feruloyl transferase are suitable targets to reduce the acetate and/or ferulate content. Targeted suppression of such transferases result in reduced formation of acetate and/or ferulate content.

In an embodiment, esterase over expression may be combined with an RNAi approach to reduce the formation of acetate and/or ferulate and thereby reducing the overall content of acetate and/or ferulate.

In an embodiment, a suppression construct to suppress the expression of a gene involved in the catalytic transfer of an acetyl or a feruloyl group to the xylosyl residues in GAX or the arabinosyl residues in GAX respectively in the Golgi apparatus.

### Example 20

### Variants of Enzyme Sequences

A. Variant Nucleotide Sequences of Esterase that do not Alter the Encoded Amino Acid Sequence

The esterase nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants are altered, the amino acid sequence encoded by the open reading frames do not change.

### B. Variant Amino Acid Sequences of Esterase Polypeptides

Variant amino acid sequences of the esterase polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

### C. Additional Variant Amino Acid Sequences of Esterase Polypeptides

In this example, artificial protein sequences are created having 80%, 85%, 90% and 95% identity relative to the reference protein sequence. This latter effort requires identifying conserved and variable regions from an alignment and then the judicious application of an amino acid substitutions table. These parts will be discussed in more detail below.

Largely, the determination of which amino acid sequences are altered is made based on the conserved regions among esterase protein or among the other esterase polypeptides. Based on the sequence alignment, the various regions of the esterase polypeptide that can likely be altered are represented in lower case letters, while the conserved regions are represented by capital letters. It is recognized that conservative substitutions can be made in the conserved regions below without altering function. In addition, one of skill will understand that functional variants of the easterase sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 5.

TABLE 5
Substitution Table

| | Strongly | | |
|---|---|---|---|
| | Similar and | Rank of | |
| Amino | Optimal | Order to | |
| Acid | Substitution | Change | (a) Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the esterase polypeptides are generating having about 80%, 85%, 90% and 95% amino acid identity to the starting unaltered ORF nucleotide sequence as claimed.

All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

What is claimed is:

1. A method of reducing acetate, arabinosidase and/or ferulate content in a plant, the method comprising expressing an enzyme that cleaves acetyl, arabinosyl or feruloyl substituents and targeting the cleaving enzyme to one or more components of the Golgi apparatus or manipulating the endogenous enzyme.

2. The method of claim 1, wherein the enzyme is an acetyl esterase, arabinosidase or feruloyl esterase.

3. The method of claim 1, wherein the plant biomass is not substantially reduced compared to a plant not expressing the esterase targeted to the Golgi.

4. The method of claim 1, wherein the enzyme targeted to Golgi is an acetyl esterase.

5. The method of claim 1 wherein the enzyme targeted is a feruloyl esterase.

6. The method of claim 1, wherein the enzyme targeted to Golgi is an arabinosidase.

7. The method of claim 1, comprising:
a. transforming a plant cell with a vector containing a polynucleotide encoding a heterologous esterase;
b. targeting the expression of said enzyme to the Golgi apparatus;
c. retaining expression of said hydrolytic enzyme in the Golgi apparatus; and
d. growing said plant under plant growing conditions.

8. The method according to claim 7, which improves composition of the biomass of a plant by overexpression of the polynucleotide.

9. The method according to claim 7, which improves ethanol production.

10. The method of claim 7, wherein the transformed plant cell further comprises one or more heterologous polynucleotides encoding a hydrolase, esterase, glycosyltransferase or arabinosidase.

11. The method of claim 7 wherein the transformed plant cell wall polysaccharides are degraded or converted to glucose, xylose, mannose, galactose, arabinose or a combination thereof at a higher rate, as compared to non-transformed plants.

12. The method of claim 7 wherein the plant cell wall acetate concentration is decreased, as compared to non-transformed plants.

13. The method of claim 7 wherein the plant cell wall feruloylation is decreased, as compared to non-transformed plants.

14. The method of claim 7 wherein the plant cell wall arabinose content is decreased, as compared to non-transformed plants.

15. The method of claim 7 wherein the plant cell wall cross-linking is decreased, as compared to non-transformed plants.

16. The method of claim 7, wherein the plant is selected from the group consisting of: maize, soybean, sunflower, sorghum, canola, wheat, alfalfa, cotton, rice, barley, millet, peanut, sugar cane, grass, turfgrass, miscanthus, switchgrass and cocoa.

17. A method of modulating plant tissue growth with a Golgi targeted enzyme in a plant, comprising expressing a recombinant expression cassette comprising the polynucleotide of claim 7 operably linked to a promoter.

18. The method of claim 16, wherein the plant is selected from the group consisting of: maize, soybean, sorghum, canola, wheat, alfalfa, cotton, rice, barley, millet, peanut, sugar cane, grass, turfgrass, miscanthus, switchgrass and cocoa.

19. The method of claim 7, wherein the plant has improved silage quality and digestibility.

20. The method of claim 7, wherein the promoter is selected from the group consisting of a leaf specific promoter, vascular element preferred promoter and a root specific promoter.

21. The method of claim 7 comprising expressing a polynucleotide that encodes a polypeptide having at least 85% sequence similarity to a polypeptide selected from the group consisting of SEQ ID NOS: 4-18, 59, 62, 65, 68, 70 and 71.

22. A transgenic plant cell of claim 7, with altered cell wall content comprising a recombinant expression cassette comprising expressing a polynucleotide that encodes a polypeptide having at least 85% sequence similarity to a polypeptide selected from the group consisting of SEQ ID NOS: 4-18, 59, 62, 65, 68, 70 and 71.

23. The transgenic plant of claim 7, wherein the plant is a monocot.

24. The transgenic plant from claim 7 where in the plant is a dicot.

25. The transgenic plant of claim 21, wherein the plant is selected from the group consisting of: maize, soybean, sunflower, sorghum, canola, grass, sugarcane, wheat, alfalfa, cotton, rice, barley, miscanthus, turfgrass, switchgrass and millet.

26. A method of modulating plant carbohydrate concentration in a transgenic plant, the method comprising expressing a recombinant polynucleotide encoding the Golgi targeting enzyme of claim 1.

27. The method of altering the cross-linking and acetyl content in plant tissues in order to improve the quality of biomass available for biofuels in a plant, the method comprising:
a. transforming a plant cell with a recombinant expression cassette comprising a polynucleotide having at least 85% sequence identity to the full length sequence of a enzyme encoding polynucleotide selected from the group consisting of SEQ ID NO: 4-18, 59, 62, 65, 68, 70 and 71, operably linked to a promoter,
b. culturing the plant cell under plant-forming conditions to express the polypeptide enzyme in the plant tissue;
c. growing the transformed plant tissue under plant tissue growing conditions; wherein the composition of the Golgi polysaccharides in said transformed plant cell is altered; and
d. processing the transformed plant tissue to obtain biofuel.

28. A method of producing biomass for silage or biofuel production comprising providing plant tissue having a substantially lowered amount of acetate or ferulate content, wherein the plant tissue expresses a recombinant esterase that is targeted to a compartment within the Golgi apparatus.

29. The method of claim 27, wherein the polypeptide comprises at least 85% sequence similarity to a polypeptide selected from the group consisting of SEQ ID NOS 4-18, 59, 62, 65, 68, 70 and 71.

30. A product derived from the method of processing of transgenic plant component expressing an isolated polynucleotide encoding a Golgi targeting enzyme, the method comprising:
a. growing a plant that expresses a polynucleotide having at least 85% sequence identity to the full length sequence of SEQ ID NO: 4-18, 59, 62, 65, 68, 70 and 71, operably linked to a promoter; and
b. processing the plant component to obtain a product.

31. A product according to claim 29, which is a constituent of ethanol.

32. A plant stover comprising a reduced acetyl or feruloyl content due to the targeting of a recombinant esterase to the Golgi apparatus, wherein the esterase catalyzes the cleavage of the acetyl or feruloyl molecules.

33. The plant stover of claim 32 is corn stover.

34. The plant stover of claim 32 is used for the production of biofuel comprising butanol.

35. The plant stover of claim 32 is used for the production of biofuel comprising ethanol.

36. A method of reducing the overall acetate and/or ferulate content in a plant tissue, the method comprising expressing an inhibitory nucleotide molecule that suppresses the expression of an acetyl or a feruloyl transferase.


SEQUENCE LISTING (71 sequences)

SEQ ID NO: 1 - protein, 70 aa, Arabidopsis thaliana
MPFSSYIGNSRRSSTGGGTGGWGQSLLPTALSKSKLAINRKPRKRTLVVNFIFANFFVIALTVSLLFFLL

SEQ ID NO: 2 - protein, 34 aa, Arabidopsis thaliana
MSKRNPKILKIFLYMLLLNSLFLIIYFVFHSSSF

SEQ ID NO: 3 - protein, 27 aa, Rattus sp.
MIHTNLKKKFSLFILVFLLFAVICVWK

SEQ ID NO: 4 - protein, 303 aa, Aspergillus ficuum
MLSTHLLFLATTLLTSLFHPIAAHVAKRSGSLQQITDFGDNPTGVGMYIYVPNNLASNPGIVVAIHYCTGTGPGYYSNSPYATLSEQYGFIVIYPSSPYSGGCWDVSSQATLTHNGGGNSNSIANMVTWTISEYGADSKKVYVTGSSSGAMMTNVMAATYPELFAAGTVYSGVSAGCFYSDTNQVDGWNSTCAQGDVITTPEHWASIAEAMYPGYSGSRPKMQIYHGSVDTTLYPQNYYETCKQWAGVFGYDYSAPESTEANTPQTNYETTIWGDNLQGIFATGVGHTVPIHGDKDMEWFGFA

SEQ ID NO: 5 - protein, 304 aa, Aspergillus niger
MLLSTHLLFVITTFLTSLLHPIAAHAVKRSGSLQQVTDFGDNPTNVGMYIYVPNNLASNPGIVVAIHYCTGTGPGYYSASPYATLSEQYGFIVIYPSSPYSGGCWDVSSQATLTHNGGGNSNSIANMVTWTISEYGADSSKVFVTGSSSGAMLTNVMAATYPELFAAATVYSGVSAGCFYSNTNQVDGWNSTCAQGDVITTPEHWASIAEAMYSGYSGSRPRMQIYHGTLHTTLYPQNYYETCKQWSGVFGYDYSAPEKTEANTPQTNYETTIWGDSLQGIFATGVGHTVPIHGDKDMEWFGFA

SEQ ID NO: 6 - protein, 307 aa, Aspergillus oryzae
MILLSYLLTYLLCALTCSARAIHNGRSLIPRAGSLEQVTDFGDNPSNVKMYIYVPTNLASNPGIIVAIHYCTGTAQAYYQGSPYAQLAETHGFIVIYPESPYEGTCWDVSSQATLTHNGGGNSNSIANMVTWTTKQYNADSSKVFVTGTSSGAMMTNVMAATYPNLFAAGVAYAGVPAGCFLSTADQPDAWNSTCAQGQSITTPEHWASIAEAMYPDYSGSRPKMQIYHGNVDTTLYPQNYEETCKQWAGVFGYNYDAPESTESNTPEANWSRTTWGPNLQGILAGGVGHNIQIHGDEDMKWFGFTN

SEQ ID NO: 7 - protein, 308 aa, Aspergillus clavatus
MAPFSFLLTLLLYTLSAGASVLESRSSALLPRAGSLQQVTNFGDNPTNVGMYIYVPNNLASNPGIIVAIHYCTGTAEAYYNGSPYAKLAEKHGFIVIYPESPYQGKCWDVSSRASLTHNGGGNSNSIANMVKWTIKKYKTNTSKVFVTGSSSGAMMTNVMAATYPDMFAAGVVYSGVAAGCFMSNTNQQAAWNSTCAHGKSIATPEAWAHVAKAMYPGYDGPRPRMQIYHGSADTTLYPQNYQETCKEWAGVFGYDYNAPRSVENNKPQANYKTTTWGKELQGIYATGVGHTVPINGDRDMAWFGFAK

SEQ ID NO: 8 - protein, 320 aa, Clostridium thermocellum
MAQLYDMPLEELKKYKPALTKQKDFDEFWEKSLKELAEIPLKYQLIPYDFPARRVKVFRVEYLGFKGANIEGWLAVPEGEGLYPGLVQFHGYNWAMDGCVPDVVNWALNGYAAFLMLVRGQQGRSVDNIVPGSGHALGWMSKGILSPEEYYYRGVYMDAVRAVEILASLPCVDESRIGVTGGSQGGGLALAVAALSGIPKVAAVHYPFLAHFERAIDVAPDGPYLEINEYLRRNSGEEIERQVKKTLSYFDIMNLAPRIKCRTWICTGLVDEITPPSTVFAVYNHLKCPKEISVFRYFGHEHMPGSVEIKLRILMDELNP

SEQ ID NO: 9 - protein, 312 aa, Neurospora crassa
MKLLSLATALLATLTTAHPVFDDLITPSTPLDHKRAPAASLRHISNFGSNPSNAKMYIYVPDNLAASPPIIVAIHYCTGTAQAYYTNSPYARLADQKGFIVIYPESPYSGTCWDVSSHATLTHNGGGNSNSIANMVEYTLKTYNGDATKVFVTGSSSGAMMTNVMAATYPALFAAGIVYSGVPAGCFYSQAGGTNAWNSSCANGQVHGTPQVWAKVVRDMYPGYDGARPKMEIYHGSADTTLNANNYNETIKQWAGVFGFDYQKPDTTQDNVPQGGYTTYTWGEGKLVGVYARGVGHSVPIRGSDDMKFFGL

SEQ ID NO: 10 - protein, 353 aa, Penicillium funiculosum
MAIPLVLVLAWLLPVVLAASLTQVNNFGDNPGSLQMYIYVPNKLASKPAIIVAMHPCGGSATEYYGMYDYHSPADQYGYILIYPSATRDYNCFDAYSSASLTHNGGSDSLSIVNMVKYVISTYGADSSKVYMTGSSSGAIMTNVLAGAYPDVFAAGSAFSGMPYACLYGAGAADPIMSNQTCSQGQIQHTGQQWAAYVHNGYPGYTGQYPRLQMWHGTADNVISYADLGQEISQWTTIMGLSFTGNQTNTPLSGYTKMVYGDGSKFQAYSAAGVGHFVPTDVSVVLDWFGITSGTTTTTTPTTTPTTSTSPSSTGGCTAAHWAQCGGIGYSGCTACASPYTCQKANDYYSQCL

SEQ ID NO: 11 - protein, 281 aa, Aspergillus niger
MKQFSAKYALILLATAGQALAASTQGISEDLYNRLVEMATISQAAYADLCNIPSTIIKGEKIYNAQTDINGWILRDDTSKEIITVFRGTGSDTNLQLDTNYTLTPFDTLPQCNDCEVHGGYYIGWISVQDQVESLVKQQASQYPDYALTVTGHSLGASMAALTAAQLSATYDNVRLYTFGEPRSGNQAFASYMNDAFQVSSPETTQYFRVTHSNDGIPNLPPADEGYAHGGVEYWSVDPYSAQNTFVCTGDEVQCCEAQGGQGVNDAHTTYFGMTSGACTW

SEQ ID NO: 12 - protein, 521 aa, Aspergillus niger
MKVASLLSLALPGAALAATDPFQSRCNEFQNKIDIANVTVRSVAYVAAGQNISQAEVASVCKASVQASVDLCRVTMNISTSDRSHLWAEAWLPRNYTGRFVSTGNGGLAGCVQETDLNFAANFGFATVGTNGGHDGDTAKYFLNNSEVLADFAYRSVHEGTVVGKQLTQLFYDEGYNYSYYLGCSTGGRQGYQQVQRFPDDYDGVIAGSAAMNFINLISWGAFLWKATGLADDPDFISANLWSVIHQEIVRQCDLVDGALDGIIEDPDFCAPVIERLICDGTTNGTSCITGAQAAKVNRALSDFYGPDGTVYYPRLNYGGEADSASLYFTGSMYSRTEEWYKYVVYNDTNWNSSQWTLESAKLALEQNPFNIQAFDPNITAFRDRGGKLLSYHGTQDPIISSTDSKLYYRRVANALNAAPSELDEFYRFFQISGMGHCGDGTGASYIGQGYGTYTSKAPQVNLLRTMVDWVENGKAPEYMPGNKLNANGSIEYMRKHCRYPKHNIHTGPGNYTDPNSWTCV

SEQ ID NO: 13 - protein, 837 aa, Clostridium thermocellum
MSRKLFSVLLVGLMLMTSLLVTISSTSAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGVESSSTGLGDLNGDGNINSSDLQALKRHLLGISPLTGEALLRADVNRSGKVDSTDYSVLKRYILRIITEFPGQGDVQTPNPSVTPTQTPIPTISGNALRDYAEARGIKIGTCVNYPFYNNSDPTYNSILQREFSMVVCENEMKFDALQPRQNVFDFSKGDQLLAFAERNGMQMRGHTLIWHNQNPSWLTNGNWNRDSLLAVMKNHITTVMTHYKGKIVEWDVANECMDDSGNGLRSSIWRNVIGQDYLDYAFRYAREADPDALLFYNDYNIEDLGPKSNAVFNMIKSMKERGVPIDGVGFQCHFINGMSPEYLASIDQNIKRYAEIGVIVSFTEIDIRIPQSENPATAFQVQANNYKELMKICLANPNCNTFVMWGFTDKYTWIPGTFPGYGNPLIYDSNYNPKPAYNAIKEALMGY

SEQ ID NO: 14 - protein, 292 aa, Neurospora crassa
MLPRTLLGLALTAATGLCASLQQVTNWGSNPTNIRMHTYVPDKLATKPAIIVALHGCGGTAPSWYSGTRLPSYADQYGFILIYPGTPNMSNCWGVNDPASLTHGAGGDSLGIVAMVNYTIAKYNADASRVYVMGTSSGGMMTNVMAATYPEVFEAGAAYSGVAHACFAGAASATPFSPNQTCARGLQHTPEEWGNFVRNSYPGYTGRRPRMQICHGLADNLVYPRCAMEALKQWSNVLGVEFSRNVSGVPSQAYTQIVYGDGSKLVGYMGAGVGHVAPTNEQVMLKFFGLIN

SEQ ID NO: 15 - protein, 503 aa, Clostridium thermocellum
MKKARMTVDKDYKIAEIDKRIYGSFVEHLGRAVYDGLYQPGNSKSDEDGFRKDVIELVKELNVPIIRYPGGNFVSNYFWEDGVGPVEDRPRRLDLAWKSIEPNQVGINEFAKWCKKVNAEIMMAVNLGTRGISDACNLLEYCNHPGGSKYSDMRIKHGVKEPHNIKVWCLGNEMDGPWQVGHKTMDEYGRIAEETARAMKMIDPSIELVACGSSSKDMPTFPQWEATVLDYAYDYVDYISLHQYYGNKENDTADFLAKSDDLDDFIRSVIATCDYIKAKKRSKKDIYLSFDEWNVWYHSNNEDANIMQNEPWRIAPPLLEDIYTFEDALLVGLMLITLMKHADRIKIACLAQLINVIAPIVTERNGGAAWRQTIFYPFMHASKYGRGIVLQPVINSPLHDTSKHEDVTDIESVAIYNEEKEEVTIFAVNRNIHEDIVLVSDVRGMKDYRLLEHIVLEHQDLKIRNSVNGEEVYPKNSDKSSFDDGILTSMLRRASWNVIRIGK

SEQ ID NO: 16 - protein, 469 aa, Bacillus subtilis
MFNRLFRVCFLAALIMAFTLPNSVYAQKPIFKEVSVHDPSIIETNGTFYVFGSHLASAKSNDLMQWQQLTTSVSNDNPLIPNVYEELKETFEWAQSDTLWAADVTQLADGKYYMYYNACRGDSPRSAMGVAVADNIEGPYKNKGIFLKSGMEGTSSDGTPYDATKHPNVVDPHTFFDKDGKLWMVYGSYSGGIFILEMNPKTGFPLPGQGYGKKLLGGNHSRIEGPYVLYNPDTQYYYLYLSYGGLDATGGYNIRVARSKKPDGPYYDAEGNPMLDVRGKGGTFFDDRSIEPYGVKLMGSYTFETENEKGTGYVSPGHNSAYYDEKTGRSYLIFHTRFPGRGEEHEVRVHQLFMNKDGWPVAAPYRYAGETLKEVKQKDITGTYKLIQHGKDISADIKQTINIQLNKNHTISGEMTGTWRKTGKNTADITLAGKKYNGVFLRQWDSVREKNVMTFSVLNTSGEAVWGSK

SEQ ID NO: 17 - protein, 506 aa, Aspergillus oryzae
MSSGLSLERACAVALGIVASASLVAAGPCDIYSSGGTPCVAAHSTARALYSAYTGALYQVKRGSDGSTTDIAPLSAGGVADAAIQDSFCANTTCLITIIYDQSGRGNHLTQAPPGGFNGPESNGYDNLASAVGAPVTLNGKKAYGVFMSPGTGYRNNAASGTATGDKAEGMYAVLDGTHYNSACCFDYGNAEVSNTDTGNGHMEAIYYGDNTVWGSGAGSGPWIMADLENGLFSGLSSTNNAGDPSISYRFVTAVVKGEANQWSIRGANAASGSLSTYYSGARPSASGYNPMSKEGAIILGIGGDNSNGAQGTFYEGVMTSGYPSDATENSVQADIVAAKYAIASLTSGPALTVGSSISLQVTTAGYTTRYLAHDGSTVNTQVVSSSSTTALRQQASWTVRTGLANSACLSFESVDTPGSYIRHYNFALLLNANDGTKQFYEDATFCPQAGLNGQGNSIRSWSYPTRYFRHYENVLYVASNGGVQTFDATTSFNDDVSWVVSTGFA

SEQ ID NO: 18 - protein, 499 aa, Aspergillus niger
MFSRRNLVALGLAATVSAGPCDIYEAGDTPCVAAHSTTRALYSSFSGALYQLQRGSDDTTTTISPLTAGGVADASAQDTFCANTTCLITIIYDQSGNGNHLTQAPPGGFDGPDVDGYDNLASAIGAPVTLNGQKAYGVFMSPGTGYRNNEATGTATGDEPEGMYAVLDGTHYNDACCFDYGNAETSSTDTGAGHMEAIYLGNSTTWGYGAGDGPWIMVDMENNLFSGADEGYNSGDPSISYSFVTAAVKGGADKWAIRGGNAASGSLSTYYSGARPDYSGYNPMSKEGAIILGIGGDNSNGAQGTFYEGVMTSGYPSDDVENSVQENIVAAKYVSGSLVSGPSFTSGEVVSLRVTTPGYTTRYIAHTDTTVNTQVVDDDSSTTLKEEASWTVVTGLANSQCFSFESVDTPGSYIRHYNFELLLNANDGTKQFHEDATFCPQAPLNGEGTSLRSWSYPTRYFRHYDNVLYAASNGGVQTFDSKTSFNNDVSFEIETAFAS

SEQ ID NO: 19 - DNA, 20 bp, artificial sequence
  primer
caccatgagtaaacggaatc

SEQ ID NO: 20 - DNA, 20 bp, artificial sequence
  primer
caccatgccgttctcctcgt

SEQ ID NO: 21 - DNA, 29 bp, artificial sequence
  primer
cccatcgataaacgacgatgagtgaaaaa

SEQ ID NO: 22 - DNA, 29 bp, artificial sequence
  primer
cccatcgatgaggaagaagaggagtgaga

SEQ ID NO: 23 - DNA, 28 bp, artificial sequence
  primer
ccatcgatatgctactatcaacccacct

SEQ ID NO: 24 - DNA, 28 bp, artificial sequence
  primer
aaatcgattcaagcaaacccaaaccact

SEQ ID NO: 25 - DNA, 28 bp, artificial sequence
  primer
ccatcgatatgatcctcttgtcatacct

SEQ ID NO: 26 - DNA, 28 bp, artificial sequence
  primer
aaatcgatctaattcgtaaacccaaacc

SEQ ID NO: 27 - DNA, 28 bp, artificial sequence
  primer
ccatcgatatggcgccgttttcattcct

SEQ ID NO: 28 - DNA, 28 bp, artificial sequence
  primer
aaatcgattcatttggcaaagccaaacc

SEQ ID NO: 29 - DNA, 29 bp, artificial sequence
  primer
ccatcgatatggcacaattatatgatatg

SEQ ID NO: 30 - DNA, 28 bp, artificial sequence
  primer
aaatcgatttacggattcagctcatcca

SEQ ID NO: 31 - DNA, 28 bp, artificial sequence
  primer
ccatcgatatgaagctcctctccctcgc

SEQ ID NO: 32 - DNA, 28 bp, artificial sequence
  primer
aaatcgatctacagcccaaagaacttca

SEQ ID NO: 33 - DNA, 28 bp, artificial sequence
  primer
ccatcgatatgaagcaattctctgcaaa

SEQ ID NO: 34 - DNA, 28 bp, artificial sequence
  primer
aaatcgatttaccaagtacaagctccgc

SEQ ID NO: 35 - DNA, 28 bp, artificial sequence
  primer
ccatcgatatgaaagtagcaagtctcct

SEQ ID NO: 36 - DNA, 28 bp, artificial sequence
  primer
aaatcgatctatacgcaagtccaggagt

SEQ ID NO: 37 - DNA, 28 bp, artificial sequence
  primer
ccatcgatatgtcaagaaaacttttcag

SEQ ID NO: 38 - DNA, 28 bp, artificial sequence
  primer
aaatcgattcaatagcccataagagctt

SEQ ID NO: 39 - DNA, 28 bp, artificial sequence
  primer
ccatcgatatgttgcccagaacattgct

SEQ ID NO: 40 - DNA, 28 bp, artificial sequence
  primer
aaatcgatctagttgatcaacccaaaga

SEQ ID NO: 41 - DNA, 28 bp, artificial sequence
  primer
ccatcgatatggcgattcccttggtcct

SEQ ID NO: 42 - DNA, 28 bp, artificial sequence
  primer
aaatcgattcacaggcactgggaataat

SEQ ID NO: 43 - DNA, 28 bp, artificial sequence
  primer
ccatcgatatgttctcccgccgaaacct

SEQ ID NO: 44 - DNA, 28 bp, artificial sequence
  primer
aaatcgatttacgaagcaaacgccgtct

SEQ ID NO: 45 - DNA, 28 bp, artificial sequence
  primer
ccatcgatatgtcctcaggattaagcct

SEQ ID NO: 46 - DNA, 28 bp, artificial sequence
  primer
aaatcgatctaagcaaagccagtgctga

SEQ ID NO: 47 - DNA, 28 bp, artificial sequence
  primer
ccatcgatatgttcaaccgattgttccg

SEQ ID NO: 48 - DNA, 28 bp, artificial sequence
  primer
aaatcgatttatttagatccccatacag

SEQ ID NO: 49 - DNA, 28 bp, artificial sequence
  primer
ccatcgatatgaaaaaagccagaatgac

SEQ ID NO: 50 - DNA, 26 bp, artificial sequence
  primer
cccaccatgaaaaaagccagaatgac

SEQ ID NO: 51 - DNA, 28 bp, artificial sequence
  primer
aaatcgatttatttacctatccgaatta

SEQ ID NO: 52 - DNA, 19 bp, artificial sequence
  primer
gtctgcaccatcgtcaacc

SEQ ID NO: 53 - DNA, 21 bp, artificial sequence
  primer
gaagtccagctgccagaaacc

SEQ ID NO: 54 - DNA, 2623 bp, Zea mays
ggaccgaggaccaaccggagccccactcggccgccgcctctcacctccaccgactgcctgcgtcccaccgctcctgctgccgcccgttgctctcgccattgaagccctcgccgcctccgcccgcttgcccgccctcccgtcggctgccttccggatgagaagcggcgcaacctgggcggtggaagcgtggtcggcctgagccttcgtgggggttagcgccgagaaggatcctggagattaagcgcatgggttccttggaggcccggtacaggccggccggggcagctgaggacacagctaagcgaaggacccagaaaagcaaaagtttcaaagaggttgagaaattcgatgtttttgtgctggagaaaagttctggttgcaaattccggtcgttgcaacttttgctcttcgctatcatgtctgctgcgtttctaacactcctatacacaccatctgtgtatgaacatcagttgcagtcaagctctcggcttgtcaacgggtggatatgggataagaggagttctgatccccgatatatatcttctgccagcattcaatgggaggatgtatataaaagcatgcaaaatgtgaatgttggtgaacaaaagctcagtgttggactcctgaattttaacaggactgagttcagtgcttggacacatatgctcccagaaagtgatttttcagtcataaggctagagcatgccaatgaaagcatcacctggcaaactctgtatcctgaatggatagatgaggaggaagaaacagagataccatcttgtccttcgcttccagatcctagtttttcaagagcgacacactttgatgttgttgctgttaagcttccctgtagtcgtgtggcgggttggtcaagagatgttgcaaggctgcatctgcagttgtcagcagcaaaattagcagcagccacagcaagaggcaatagaggaatccatgtgctgtttgtgactgattgcttcccaattccaaacctcttctcttgcaaggacctagtgaaacgtgaaggcaatgcttggatgtacaaacctgacgtgaaggctctaaaggagaagctcaggctgcctgttggttcctgtgagcttgctgttccactcaacgcaaaagcacgactctacacggtagacagacgcagagaagcatatgctacaatacttcattcagcaagtgaatatgtttgcggtgcgataacagcagctcaaagcattcgtcaagcaggatcaacaagagaccttgttattcttgttgatgacaccataagtgaccaccaccgcaaggggctggaatctgctgggtggaaggtcagaataatacaaaggatccggaatcccaaagccgaacgtgatgcctacaacgaatggaactacagcaaattccggctgtggcagcttacagattacgacaaggtcattttcattgatgctgatctgctcatcctgaggaacattgatttcttgtttgcaatgccagaaatcaccgcaactgggaacaatgctacactcttcaactctggggtgatggtcattgagccttcaaactgcacgttccagttactgatggagcacatcaacgagataacatcttacaacggtggtgaccaagggtacctgaatgagatattcacatggtggcaccggattccaaagcacatgaatttcctgaagcatttctgggagggtgatgaggacgaagtgaaggcgaagaagactcggctgttcggcgccaacccaccgatcctctacgttctccactacttggggcggaagccatggctgtgcttccgggactacgattgcaactggaacgtcgagatcttgcgggaattcgcgagtgacgttgcgcatgcccgctggtggaaggtgcacaacaagatgccgaagaagcttcagagctattgcctcctgaggtcaaggctgaaggctgggctggagtgggagcggcggcaggccgagaaggcgaacttcaccgacgggcattggaaacggaacataaccgatccgaggctgaagacttgcttcgagaagttctgcttctgggagagcatgctatggcactggggcgagaacaagaacaactcgacgcagagcagcgcggtgccggcaacacctgccgcgacgagcctgtcgagctcgtgaggcctgttgtgtagatacagctttgttgagagtagtagtataccagatacgaaacttctgaagctcctccatacatacatagcaacagctctgtaaaggtagctatgtagaagccttttccccccgaatgactatactcttgttcgccgtcacggctgcagcgccccctcctgctgcttcctgatggctgacaattcttttggtttttgccaataattcatcagtataggctgtttttcactctgggcggcttgaggtccggtctggggctgttgttgcttcaaaccctaccggggctactgcttttgctgcggtacgccagcggtttgatgtatggtggttgaacagtttcagtgataaatctggagtgatgcaatatgggattgctgaccagcaaacactgcatcatctatggcatgaatatttatttgatcggttattgtctgaaaaaaaaaaaaaaaaaaaaaaaaaaa

SEQ ID NO: 55 - DNA, 1905 bp, Zea mays
atgggttccttggaggcccggtacaggccggccggggcagctgaggacacagctaagcgaaggacccagaaaagcaaaagtttcaaagaggttgagaaattcgatgtttttgtgctggagaaaagttctggttgcaaattccggtcgttgcaacttttgctcttcgctatcatgtctgctgcgtttctaacactcctatacacaccatctgtgtatgaacatcagttgcagtcaagctctcggcttgtcaacgggtggatatgggataagaggagttctgatccccgatatatatcttctgccagcattcaatgggaggatgtatataaaagcatgcaaaatgtgaatgttggtgaacaaaagctcagtgttggactcctgaattttaacaggactgagttcagtgcttggacacatatgctcccagaaagtgatttttcagtcataaggctagagcatgccaatgaaagcatcacctggcaaactctgtatcctgaatggatagatgaggaggaagaaacagagataccatcttgtccttcgcttccagatcctagtttttcaagagcgacacactttgatgttgttgctgttaagcttccctgtagtcgtgtggcgggttggtcaagagatgttgcaaggctgcatctgcagttgtcagcagcaaaattagcagcagccacagcaagaggcaatagaggaatccatgtgctgtttgtgactgattgcttcccaattccaaacctcttctcttgcaaggacctagtgaaacgtgaaggcaatgcttggatgtacaaacctgacgtgaaggctctaaaggagaagctcaggctgcctgttggttcctgtgagcttgctgttccactcaacgcaaaagcacgactctacacggtagacagacgcagagaagcatatgctacaatacttcattcagcaagtgaatatgtttgcggtgcgataacagcagctcaaagcattcgtcaagcaggatcaacaagagaccttgttattcttgttgatgacaccataagtgaccaccaccgcaaggggctggaatctgctgggtggaaggtcagaataatacaaaggatccggaatcccaaagccgaacgtgatgcctacaacgaatggaactacagcaaattccggctgtggcagcttacagattacgacaaggtcattttcattgatgctgatctgctcatcctgaggaacattgatttcttgtttgcaatgccagaaatcaccgcaactgggaacaatgctacactcttcaactctggggtgatggtcattgagccttcaaactgcacgttccagttactgatggagcacatcaacgagataacatcttacaacggtggtgaccaagggtacctgaatgagatattcacatggtggcaccggattccaaagcacatgaatttcctgaagcatttctgggagggtgatgaggacgaagtgaaggcgaagaagactcggctgttcggcgccaacccaccgatcctctacgttctccactacttggggcggaagccatggctgtgcttccgggactacgattgcaactggaacgtcgagatcttgcgggaattcgcgagtgacgttgcgcatgcccgctggtggaaggtgcacaacaagatgccgaagaagcttcagagctattgcctcctgaggtcaaggctgaaggctgggctggagtgggagcggcggcaggccgagaaggcgaacttcaccgacgggcattggaaacggaacataaccgatccgaggctgaagacttgcttcgagaagttctgcttctgggagagcatgctatggcactggggcgagaacaagaacaactcgacgcagagcagcgcggtgccggcaacacctgccgcgacgagcctgtcgagctcgtga

SEQ ID NO: 56 - protein, 634 aa, Zea mays
MGSLEARYRPAGAAEDTAKRRTQKSKSFKEVEKFDVFVLEKSSGCKFRSLQLLLFAIMSAAFLTLLYTPSVYEHQLQSSSRLVNGWIWDKRSSDPRYISSASIQWEDVYKSMQNVNVGEQKLSVGLLNFNRTEFSAWTHMLPESDFSVIRLEHANESITWQTLYPEWIDEEEETEIPSCPSLPDPSFSRATHFDVVAVKLPCSRVAGWSRDVARLHLQLSAAKLAAATARGNRGIHVLFVTDCFPIPNLFSCKDLVKREGNAWMYKPDVKALKEKLRLPVGSCELAVPLNAKARLYTVDRRREAYATILHSASEYVCGAITAAQSIRQAGSTRDLVILVDDTISDHHRKGLESAGWKVRIIQRIRNPKAERDAYNEWNYSKFRLWQLTDYDKVIFIDADLLILRNIDFLFAMPEITATGNNATLFNSGVMVIEPSNCTFQLLMEHINEITSYNGGDQGYLNEIFTWWHRIPKHMNFLKHFWEGDEDEVKAKKTRLFGANPPILYVLHYLGRKPWLCFRDYDCNWNVEILREFASDVAHARWWKVHNKMPKKLQSYCLLRSRLKAGLEWERRQAEKANFTDGHWKRNITDPRLKTCFEKFCFWESMLWHWGENKNNSTQSSAVPATPAATSLSSS

SEQ ID NO: 57 - DNA, 2852 bp, Zea mays
gctctgtagaagagaggggaggaaggaccgaggaccaaccggagccccactcggccgccgcctctcacctccaccgactgcctgcgtcccaccgctcctgctgccgcccgttgctctcgccattgaagccctcgccgcctccgcccgcttgcccgccctcccgtcggctgccttccggatgagaagcggcgcaacctgggcggtggaagcgtggtcggactgagccttcgtgggggttagcgccgagaaggatcctggagattaagcgcatgggttccttggaggcccggtacaggccgaccggggcagctgaggacacagctaagcgaaggacccagaaaagcaaaagtttcaaagaggttgagaaatttgatgtttttgtgctggagaaaagttctggttgcaaattccggtcgttgcaacttttgctcttcgctatcatgtctgctgcgtttctaacactcctatacacaccatctgtgtatgaacatcagttgcagtcaagctctcggcttgtcaatgggtggatatgggataagaggagttctgatccccgatatatatcttctgccagcattcaatgggaggatgtatataaaagcatgcaaaatctgaatgttggtgaacaaaagctcagtgttggactcctgaattttaacaggactgagttcagtgcttggacacatatgctcccagaaagtgatttttcagtcataaggctagagcatgccaatgaaagcatcacctggcaaactctgtatcctgaatggatagatgaggaggaagaaacagagataccatcttgtccttcgcttccagatcctagtttttcaagagcgacacactttgatgttgttgctgttaagcttccctgtactcgtgtggcgggttggtcaagagatgttgcaaggctgcatctgcagttgtcagcagcaaaattagcagcagccacagcaagaggcaatagaggaatccatgtgctgtttgtgactgattgcttcccaattccaaacctcttctcttgcaaggacctagtgaaacgtgaaggcaatgcttggatgtacaaacctgacgtgaaggctctaaaggagaagctcaggctgcctgttggttcctgtgagcttgctgttccactcaacgcaaaagcacgactctacacggtagacagacgcagagaagcatatgctacaatactgcattcagcaagtgaatatgtttgcggtgcgataacagcagctcaaagcattcgtcaagcaggatcaacaagggaccttgttattcttgttgatgacaccataagcgaccaccaccgcaaggggctggaatctgctgggtggaaggttagaataatacagaggatccggaatcccaaagccgagcgtgatgcctacaacgaatggaactacagcaaattccggctgtggcagcttacagattatgacaaggtcattttcattgatgctgatctgctcatcctgaggaacattgatttcttgtttgcaatgccggaaatcaccgcaactgggaacaatgcaacactcttcaactctggagtgatggtcattgagccttcaaactgcacgttccagttactgatggagcacatcaacgagataacatcttacaacggtggtgaccaagggtacctgaatgagatattcacatggtggcaccggattccaaagcacatgaatttcttgaagcatttctgggagggtgacgaggacgaagtgaaggccaagaagactcggctgttcggcgccaacccaccgatcctctacgttctccactacttggggcggaagccatggctgtgcttccgggactacgattgcaactggaacgttgagatcttgcgggagtttgcgactgatgttgcgcacgcccgctggtggaaggtgcacaacaagatgccgaagaagcttcagagctattgcctcctgaggtcaaggctgaaggctgggttggagtgggagcggcggcaggccgagaaggcgaacttcaccgacgggcattggaaacggaacataaccgatccgaggctgaagacttgcttcgagaagttctgcttctgggagagcatgctatggcactggggcgagaacaagaacaactcgacgcagagcagcgcggtgccggcaacacctgccgcgacgagcctgtcgagctcgtgaggcctgttgtgtagatacagctttgttgagagtagtataccagatacgaaacttctgaagctcctccatacatacatagcaacagctctgtaaagcactcgtgtttgtttctggtcgtgtgccaccaagccatggacgcaatgcaagcgttcaaatgccgtgcctccctgcgtcgtactggcacagagcaagcatcatcattgcgtccatcgagagctttggttccgtgcactgctgctgttgcgccaaagaacgagacgcatcattgcccggatccatgatctcgtggtgctgtcgagttccttggaggtgcggtcttttgcagctcatcagcctgctcgcttttccagcagcggcacacctaaaaagcacagggataggctgtatctgcgtgcgacctcgctccggcggtgccgtcgcttcgcctgagacagagacactgtacgtacacctggttgggtccttcccttgtcgctcacgattggagtagttggatgtttgcgttgcaagtgactgagattaggttttctccacgtttgccactacaaaatccgcgagatgcgcgtccagatctgacgcgcagtctagtagtgctacagtaatataatgtatagaaaaaactattgcgcgattaagttttttcgtgcgatcatcgtgtagttaaatgactgaaaattt

SEQ ID NO: 58 - DNA, 1905 bp, Zea mays
atgggttccttggaggcccggtacaggccgaccggggcagctgaggacacagctaagcgaaggacccagaaaagcaaaagtttcaaagaggttgagaaatttgatgtttttgtgctggagaaaagttctggttgcaaattccggtcgttgcaacttttgctcttcgctatcatgtctgctgcgtttctaacactcctatacacaccatctgtgtatgaacatcagttgcagtcaagctctcggcttgtcaatgggtggatatgggataagaggagttctgatccccgatatatatcttctgccagcattcaatgggaggatgtatataaaagcatgcaaaatctgaatgttggtgaacaaaagctcagtgttggactcctgaattttaacaggactgagttcagtgcttggacacatatgctcccagaaagtgatttttcagtcataaggctagagcatgccaatgaaagcatcacctggcaaactctgtatcctgaatggatagatgaggaggaagaaacagagataccatcttgtccttcgcttccagatcctagtttttcaagagcgacacactttgatgttgttgctgttaagcttccctgtactcgtgtggcgggttggtcaagagatgttgcaaggctgcatctgcagttgtcagcagcaaaattagcagcagccacagcaagaggcaatagaggaatccatgtgctgtttgtgactgattgcttcccaattccaaacctcttctcttgcaaggacctagtgaaacgtgaaggcaatgcttggatgtacaaacctgacgtgaaggctctaaaggagaagctcaggctgcctgttggttcctgtgagcttgctgttccactcaacgcaaaagcacgactctacacggtagacagacgcagagaagcatatgctacaatactgcattcagcaagtgaatatgtttgcggtgcgataacagcagctcaaagcattcgtcaagcaggatcaacaagggaccttgttattcttgttgatgacaccataagcgaccaccaccgcaaggggctggaatctgctgggtggaaggttagaataatacagaggatccggaatcccaaagccgagcgtgatgcctacaacgaatggaactacagcaaattccggctgtggcagcttacagattatgacaaggtcattttcattgatgctgatctgctcatcctgaggaacattgatttcttgtttgcaatgccggaaatcaccgcaactgggaacaatgcaacactcttcaactctggagtgatggtcattgagccttcaaactgcacgttccagttactgatggagcacatcaacgagataacatcttacaacggtggtgaccaagggtacctgaatgagatattcacatggtggcaccggattccaaagcacatgaatttcttgaagcatttctgggagggtgacgaggacgaagtgaaggccaagaagactcggctgttcggcgccaacccaccgatcctctacgttctccactacttggggcggaagccatggctgtgcttccgggactacgattgcaactggaacgttgagatcttgcgggagtttgcgactgatgttgcgcacgcccgctggtggaaggtgcacaacaagatgccgaagaagcttcagagctattgcctcctgaggtcaaggctgaaggctgggttggagtgggagcggcggcaggccgagaaggcgaacttcaccgacgggcattggaaacggaacataaccgatccgaggctgaagacttgcttcgagaagttctgcttctgggagagcatgctatggcactggggcgagaacaagaacaactcgacgcagagcagcgcggtgccggcaacacctgccgcgacgagcctgtcgagctcgtga

SEQ ID NO: 59 - protein, 634 aa, Zea mays
MGSLEARYRPTGAAEDTAKRRTQKSKSFKEVEKFDVFVLEKSSGCKFRSLQLLLFAIMSAAFLTLLYTPSVYEHQLQSSSRLVNGWIWDKRSSDPRYISSASIQWEDVYKSMQNLNVGEQKLSVGLLNFNRTEFSAWTHMLPESDFSVIRLEHANESITWQTLYPEWIDEEEETEIPSCPSLPDPSFSRATHFDVVAVKLPCTRVAGWSRDVARLHLQLSAAKLAAATARGNRGIHVLFVTDCFPIPNLFSCKDLVKREGNAWMYKPDVKALKEKLRLPVGSCELAVPLNAKARLYTVDRRREAYATILHSASEYVCGAITAAQSIRQAGSTRDLVILVDDTISDHHRKGLESAGWKVRIIQRIRNPKAERDAYNEWNYSKFRLWQLTDYDKVIFIDADLLILRNIDFLFAMPEITATGNNATLFNSGVMVIEPSNCTFQLLMEHINEITSYNGGDQGYLNEIFTWWHRIPKHMNFLKHFWEGDEDEVKAKKTRLFGANPPILYVLHYLGRKPWLCFRDYDCNWNVEILREFATDVAHARWWKVHNKMPKKLQSYCLLRSRLKAGLEWERRQAEKANFTDGHWKRNITDPRLKTCFEKFCFWESMLWHWGENKNNSTQSSAVPATPAATSLSSS

SEQ ID NO: 60 - DNA, 2501 bp, Zea mays
aagcccacacagagctctgcacaagaaaaggggaggaaggacgagccggagccactcagccgccacctcaccccccccatcggccgcctgcgtcccaccacccgctcctgctgccgggcctgccgcccgttgctctcgccactgaagccctcgccgccgcctccgcctccgcccgcttgcttgccctcctgccggctgcctcccggatgagaagcggcgcaacctgggcagcggaagcgtggtcggcctgagccttcgccggggtttctgccgagaaggatcctggagattaagcgcatgggttccttggaggcccggtacaggccggccggggcagctgaggacacagctaagagaaggacccaaaaaagcaaaagtttcaaagaggttgaaaaatttgatgtttttgtgctggagaaaagttctggttgcaagttccgatcgatgcaacttttgctcttcgctatcatgtctgctgcatttctaacaatcctatacacaccatctgtgtatgaacatcagttgcagtcaagctctcggcttgtcaatgggtggatatgggataagaggagttctgatccccgatatgtatcttctgctagcattcaatgggaggatgtatataaaagtatccaaaatctgaatgttggtgaacagaagctcagtgttggactcctgaattttaacaggactgagttcgacgcttggacacacatgctcccagaaagtgatttctcaatcataaggctggagcatgccaatgaaagcattacctggcaaactctctatcctgaatggatagatgaggaggaagaaactgagataccatcttgcccttcgcttccagatccaagtttcccaagagcaacacattttgatgttattgctgttaagctcccctgttctcgtgtggctggttggtcaagagatgttgcaaggctacatttgcagttatcagcagcaaaattagcagcgaccacagcaagaggcaatagtggaatccatgtgctgtttgtgactgattgcttcccgattccgaacctcttctcttgcaaggacctagtgaagcgtgaaggcaatgcttggatgtacaaacctgacgtgaaggcgttgaaggagaagctcaggctgcctgttggttcctgtgagcttgctgttccactcaacgcaaaagcacgactctacacagtagacagacgcagagaagcatatgcgacaatactgcattcagcaagtgaatatgtttgcggcgcgatcacggcagctcaaagcattcgtcaggcaggatcaacaagagacctagttattctcgtcgacgacaccataagtgaccaccaccgcaaggggctggaatctgcggggtggaaggtcaggataatacagaggatccggaaccccaaagccgagcgcgacgcctacaacgagtggaactacagcaaattccggctgtggcagctcacggattacgacaaggtcatcttcatcgacgcggatctcctcatcctgaggaacatcgatttcctgttcgcgctgccggagatcacggcgacggggaacaacgcgacgctcttcaactcgggagtgatggtcatcgagccttcgaactgcacgttccggctactgatggagcacatcgacgagataacgtcgtacaacggcggggaccaggggtacctgaacgagatattcacgtggtggcaccggatcccgaagcacatgaacttcctgaagcatttctgggagggcgacgaggaggaggtgaaggcgaagaagacccggctgttcggcgcgaacccgccggtcctgtacgtgctccactacctggggaggaagccgtggctgtgcttccgggactacgactgcaactggaacgtggagatcctgcgggagttcgcgagcgacgtcgcgcacgcccgctggtggaaggtgcacaaccggatgcccaggaagctccagagctactgccttctgaggtcgagcctgaaggccgggctggagtgggagcggcggcaggccgagaaggcgaacttcacggacgggcactggaagcggaacgtaacggacccgaggctgaagacctgcttcgagaagttctgcttctgggagagcatgctgtggcactggggcgagaagagcaagagcaactcgacgacgacgcggaacagcgccgtgccggcagcaacgacaacgcctgctgcgagcctgtcgagctcgtgagacttgtagatagctttgtctgccgagagtagtataccagtaccagatacagaacttctgaagctctccatacatacatagcgacagctctgtaaaggtagctatgtaggccttttccttccccgaatgactatataccttcgtcttcgttcgccgtcacagctgcaggcagctccctccctccccctggtttccgatggttaacaattcctttttttttgccaataattcatcagtataggatgtccggctatgttgcctcaaaaaaaaaaaaaaaaaaaaaa

SEQ ID NO: 61 - DNA, 1920 bp, Zea mays
atgggttccttggaggcccggtacaggccggccggggcagctgaggacacagctaagagaaggacccaaaaaagcaaaagtttcaaagaggttgaaaaatttgatgtttttgtgctggagaaaagttctggttgcaagttccgatcgatgcaacttttgctcttcgctatcatgtctgctgcatttctaacaatcctatacacaccatctgtgtatgaacatcagttgcagtcaagctctcggcttgtcaatgggtggatatgggataagaggagttctgatccccgatatgtatcttctgctagcattcaatgggaggatgtatataaaagtatccaaaatctgaatgttggtgaacagaagctcagtgttggactcctgaattttaacaggactgagttcgacgcttggacacacatgctcccagaaagtgatttctcaatcataaggctggagcatgccaatgaaagcattacctggcaaactctctatcctgaatggatagatgaggaggaagaaactgagataccatcttgcccttcgcttccagatccaagtttcccaagagcaacacattttgatgttattgctgttaagctcccctgttctcgtgtggctggttggtcaagagatgttgcaaggctacatttgcagttatcagcagcaaaattagcagcgaccacagcaagaggcaatagtggaatccatgtgctgtttgtgactgattgcttcccgattccgaacctcttctcttgcaaggacctagtgaagcgtgaaggcaatgcttggatgtacaaacctgacgtgaaggcgttgaaggagaagctcaggctgcctgttggttcctgtgagcttgctgttccactcaacgcaaaagcacgactctacacagtagacagacgcagagaagcatatgcgacaatactgcattcagcaagtgaatatgtttgcggcgcgatcacggcagctcaaagcattcgtcaggcaggatcaacaagagacctagttattctcgtcgacgacaccataagtgaccaccaccgcaaggggctggaatctgcggggtggaaggtcaggataatacagaggatccggaaccccaaagccgagcgcgacgcctacaacgagtggaactacagcaaattccggctgtggcagctcacggattacgacaaggtcatcttcatcgacgcggatctcctcatcctgaggaacatcgatttcctgttcgcgctgccggagatcacggcgacggggaacaacgcgacgctcttcaactcgggagtgatggtcatcgagccttcgaactgcacgttccggctactgatggagcacatcgacgagataacgtcgtacaacggcggggaccaggggtacctgaacgagatattcacgtggtggcaccggatcccgaagcacatgaacttcctgaagcatttctgggagggcgacgaggaggaggtgaaggcgaagaagacccggctgttcggcgcgaacccgccggtcctgtacgtgctccactacctggggaggaagccgtggctgtgcttccgggactacgactgcaactggaacgtggagatcctgcgggagttcgcgagcgacgtcgcgcacgcccgctggtggaaggtgcacaaccggatgcccaggaagctccagagctactgccttctgaggtcgagcctgaaggccgggctggagtgggagcggcggcaggccgagaaggcgaacttcacggacgggcactggaagcggaacgtaacggacccgaggctgaagacctgcttcgagaagttctgcttctgggagagcatgctgtggcactggggcgagaagagcaagagcaactcgacgacgacgcggaacagcgccgtgccggcagcaacgacaacgcctgctgcgagcctgtcgagctcgtga

SEQ ID NO: 62 - protein, 639 aa, Zea mays
MGSLEARYRPAGAAEDTAKRRTQKSKSFKEVEKFDVFVLEKSSGCKFRSMQLLLFAIMSAAFLTILYTPSVYEHQLQSSSRLVNGWIWDKRSSDPRYVSSASIQWEDVYKSIQNLNVGEQKLSVGLLNFNRTEFDAWTHMLPESDFSIIRLEHANESITWQTLYPEWIDEEEETEIPSCPSLPDPSFPRATHFDVIAVKLPCSRVAGWSRDVARLHLQLSAAKLAATTARGNSGIHVLFVTDCFPIPNLFSCKDLVKREGNAWMYKPDVKALKEKLRLPVGSCELAVPLNAKARLYTVDRRREAYATILHSASEYVCGAITAAQSIRQAGSTRDLVILVDDTISDHHRKGLESAGWKVRIIQRIRNPKAERDAYNEWNYSKFRLWQLTDYDKVIFIDADLLILRNIDFLFALPEITATGNNATLFNSGVMVIEPSNCTFRLLMEHIDEITSYNGGDQGYLNEIFTWWHRIPKHMNFLKHFWEGDEEEVKAKKTRLFGANPPVLYVLHYLGRKPWLCFRDYDCNWNVEILREFASDVAHARWWKVHNRMPRKLQSYCLLRSSLKAGLEWERRQAEKANFTDGHWKRNVTDPRLKTCFEKFCFWESMLWHWGEKSKSNSTTTRNSAVPAATTTPAASLSSS

SEQ ID NO: 63 - DNA, 2554 bp, Zea mays
tatatagagcttcgtcccctcccgagctcgcacaccgagttcttcttcttaatcttcctcttcgtctccttctagtagtgggcagagagagatcgagtgctaggggagaagcaagcaagccaggcagcagcaaccgcgcctggtttcctcctcctcctcctcgtgggctcacacaaggcttccttcggttcgggggtgtgcgaagcggcgcaacggcggaggcggctctagcatcaccggccgacggtcctcctccttctcacgaaaggcactagaggagcggcggcggcggcggcgcatcaagcaccgcaccgatgggcccactggagccgcgctaccgtccaggcggcgcccccgaggacacgactaagagaagggcctccaagagcaagagtttcaaagacgccgagaacttcgaagtcctcgtccttgagaagagctgcggttgcaagttcaagtctttgaggatcttgctcatagccatcatctccgcgacagtccttacccttgtaaccccgaccttatacgagcgccagttgcagtcagcctcccggtacgtggatgtcgggtggatgtgggacagagcgagttccgatccgcggtacgcatcttctgccgatgttgggtgggaggatgtgtacaaagcactgggaaacctaagaagcggtaaccgccagagtcatctcagagttgggctcttgaatttcaacagcaccgagtatggctcctggacgcagttgctcccagctgacagccacgttatctccactgtaaggctcgagcacgccaaggacagcgtcacctggcagacgctgtaccctgagtggatcgacgaggaggaagagacggagataccctcttgcccgtcgctgccggagccaaacgtgccaagaggtgcgcgctttgacgtcgtcgccgtgaagctcccatgcacccgtgtggcgggctggtcgagagacgtcgcgcggctccatctgcagctctcggcagccaaactggctgtggcgtcctcgaagcgcaaccacgacgtccatgttctcttcgtcactgactgcttcccgatcccgaacctcttcccttgcaagaaccttgtcacacgtgaaggcagcgcctggttgtacagtcctgactccaaggcgttgagggaaaagctcaggcttccagtcgggtcctgtgagcttgccgttccactcaaagccaaatcgaggcttttctcggtagatcgacgaagagaagcgtacgcgacgatactgcattcagcgagcgaatacgtctgcggcgcaatctcggcagcgcaaagcatccgccaggcaggatccaccagggacctggtcatccttgtggacgagaccataagcgaccaccaccggagaggcttggaggcggcggggtggaaggtcagagtgatccagaggatcaggaaccccaaggcggagcgcgacgcgtacaacgagtggaactacagcaagttcaggctgtggcagctcaccgactacgacaaggtcatcttcatagacgccgacctcctcatcctgaggaacgtcgacttcctgttcgccatgccggagatcgccgcgacgggcaacaacgccacgctcttcaactccggcgtcatggtcgtcgagccctccaactgcacgttccgcctgctcatggaccacatcgacgagatcacctcgtacaacggcggggaccaggggtacctcaacgagatattcacgtggtggcaccgcgtccccaggcacatgaacttcctcaagcacttctgggagggcgacagcgaggccatgaaggcgaagaagacccagctgttcggtgcggacccgccggtcctctacgtcctccactaccttggcctcaagccgtggctgtgcttcagagactacgactgcaactggaacaacgccgggatgcgcgagttcgccagcgacgtcgcgcatgcccggtggtggaaggtgcacgacaggatgccccggaagctccagtcctactgcctgctgaggtcgcggcagaaggccaggctggagtgggaccggaggcaggccgagaaggccaactctcaagatggccactggcgcctcaacgtcacggacaccaggctcaagacgtgctttgagaagttctgcttctgggagagcatgctctggcattggggcgagaacagtaacaggaccaagagcgtccccatggcagccacgacggcaaggtcgtgatctgtagatatacgaacaccccatccccatatggcaaccatacatgcatagcaatagcttgtataggtagctatgctttagttcttcgctatatatacagaatacaccactcgatccctgttgttgtcaaggctgcagctctatgtcgctgccggcctgccaccatggctaacgattcttttgggttggctgctgtaataagtttcaggtacatgtaaatttccctgctgaaattacgtgaccgcgtgtgagaaatgaatttgtacagggcgccaaataataattggttggtgcatacaacatatgaccagtcttttgcagtaaaaaaaaaaaaaaaaaaa

SEQ ID NO: 64 - DNA, 1899 bp, Zea mays
atgggcccactggagccgcgctaccgtccaggcggcgcccccgaggacacgactaagagaagggcctccaagagcaagagtttcaaagacgccgagaacttcgaagtcctcgtccttgagaagagctgcggttgcaagttcaagtctttgaggatcttgctcatagccatcatctccgcgacagtccttacccttgtaaccccgaccttatacgagcgccagttgcagtcagcctcccggtacgtggatgtcgggtggatgtgggacagagcgagttccgatccgcggtacgcatcttctgccgatgttgggtgggaggatgtgtacaaagcactgggaaacctaagaagcggtaaccgccagagtcatctcagagttgggctcttgaatttcaacagcaccgagtatggctcctggacgcagttgctcccagctgacagccacgttatctccactgtaaggctcgagcacgccaaggacagcgtcacctggcagacgctgtaccctgagtggatcgacgaggaggaagagacggagataccctcttgcccgtcgctgccggagccaaacgtgccaagaggtgcgcgctttgacgtcgtcgccgtgaagctcccatgcacccgtgtggcgggctggtcgagagacgtcgcgcggctccatctgcagctctcggcagccaaactggctgtggcgtcctcgaagcgcaaccacgacgtccatgttctcttcgtcactgactgcttcccgatcccgaacctcttcccttgcaagaaccttgtcacacgtgaaggcagcgcctggttgtacagtcctgactccaaggcgttgagggaaaagctcaggcttccagtcgggtcctgtgagcttgccgttccactcaaagccaaatcgaggcttttctcggtagatcgacgaagagaagcgtacgcgacgatactgcattcagcgagcgaatacgtctgcggcgcaatctcggcagcgcaaagcatccgccaggcaggatccaccagggacctggtcatccttgtggacgagaccataagcgaccaccaccggagaggcttggaggcggcggggtggaaggtcagagtgatccagaggatcaggaaccccaaggcggagcgcgacgcgtacaacgagtggaactacagcaagttcaggctgtggcagctcaccgactacgacaaggtcatcttcatagacgccgacctcctcatcctgaggaacgtcgacttcctgttcgccatgccggagatcgccgcgacgggcaacaacgccacgctcttcaactccggcgtcatggtcgtcgagccctccaactgcacgttccgcctgctcatggaccacatcgacgagatcacctcgtacaacggcggggaccaggggtacctcaacgagatattcacgtggtggcaccgcgtccccaggcacatgaacttcctcaagcacttctgggagggcgacagcgaggccatgaaggcgaagaagacccagctgttcggtgcggacccgccggtcctctacgtcctccactaccttggcctcaagccgtggctgtgcttcagagactacgactgcaactggaacaacgccgggatgcgcgagttcgccagcgacgtcgcgcatgcccggtggtggaaggtgcacgacaggatgccccggaagctccagtcctactgcctgctgaggtcgcggcagaaggccaggctggagtgggaccggaggcaggccgagaaggccaactctcaagatggccactggcgcctcaacgtcacggacaccaggctcaagacgtgctttgagaagttctgcttctgggagagcatgctctggcattggggcgagaacagtaacaggaccaagagcgtccccatggcagccacgacggcaaggtcgtga

SEQ ID NO: 65 - protein, 632 aa, Zea mays
MGPLEPRYRPGGAPEDTTKRRASKSKSFKDAENFEVLVLEKSCGCKFKSLRILLIAIISATVLTLVTPTLYERQLQSASRYVDVGWMWDRASSDPRYASSADVGWEDVYKALGNLRSGNRQSHLRVGLLNFNSTEYGSWTQLLPADSHVISTVRLEHAKDSVTWQTLYPEWIDEEEETEIPSCPSLPEPNVPRGARFDVVAVKLPCTRVAGWSRDVARLHLQLSAAKLAVASSKRNHDVHVLFVTDCFPIPNLFPCKNLVTREGSAWLYSPDSKALREKLRLPVGSCELAVPLKAKSRLFSVDRRREAYATILHSASEYVCGAISAAQSIRQAGSTRDLVILVDETISDHHRRGLEAAGWKVRVIQRIRNPKAERDAYNEWNYSKFRLWQLTDYDKVIFIDADLLILRNVDFLFAMPEIAATGNNATLFNSGVMVVEPSNCTFRLLMDHIDEITSYNGGDQGYLNEIFTWWHRVPRHMNFLKHFWEGDSEAMKAKKTQLFGADPPVLYVLHYLGLKPWLCFRDYDCNWNNAGMREFASDVAHARWWKVHDRMPRKLQSYCLLRSRQKARLEWDRRQAEKANSQDGHWRLNVTDTRLKTCFEKFCFWESMLWHWGENSNRTKSVPMAATTARS

SEQ ID NO: 66 - DNA, 2240 bp, Zea mays
gttgatcatatccacatatatagctatagcttgccggaggatcaagggaccatatatatacatacatacagtcagtctactgatcgtcgacgacgatcgacaaagctcgccttctcttgcctttcctgctgcttgttgctgcacaagatcatcaggcgacaccgaggattttgttcatctcagctagctagctagctacacctcctcttggcgcatcaagatgaggggtttcgtcgcttgcgccgttgcagagaaacgccaccgcttggacaggacattatttagcggtttgagcaagaaggactgcatcgggaggtactacgccaaggatgccaccaagtacaggccgttcagcgcgctgctgcccgaggggggctggagcggcaaggtgctgtacgtcaagctcgtgctcgtcctcctcatgtgcggctccttcatgggcctcctcaactcgccgtccatccacctcgcggacgaacgtcaccaccacgcgcggactgcagaggcgtggaatgcgagctggacgtcgcaccccgacgcggcgaactccgggtacgcgtcgagcctgaggatcgactggtcgcaggtccagacagcggcgaagcaggtcgctccgccggcgggcggcggcgccacgcgtgtggcgctgctcaacttcgacgacggcgaggtccaggagtggaaggcgcggatgccgcacacggacgcctccacggtgcgcctggaccacgtcgggagcgacgtcacctgggaccacctgtacccggagtggatcgacgaggaggagcactacggcgcgccggcgtgcccggacctgccggagcctaaggtggccaaggaggaggaggcgtacgacgtcgtcgcggtcaagctgccttgcgggcgcgcggcgagctggtccaaggacgtggcgcgcctgcacctgcagctggccgcggcgcgtctcgcggccgcgcgcgcgccccgcggcggcggcgggcaggcggcgcacgtgctcgtggtcagccggtgcttcccgacgcccaacctgttcaggtgccgggacgaggtggcgcgccacggcgacgtgtggctgtacaggccggacgtgggcgacctcacccggaagctggagctccccgtggggtcctgcaagctggccatgccatccaaagcgctaggcgaacattacgcgtcggcggcgccgcagcgcgaggcgtacgccacgatcctccactcggagcagctgtacgcgtgcggcgccgtcacggcggcgcggagcatccggatggcggggtccgggcgggacatggtggccctggtggacgagacgatcagcgcgcggcaccgcgccgccctggaggcggccgggtggaaggtgcggacgatccggcgcatccgcaacccgcgggcgtcgcgggacgcgtacaacgagtggaactacagcaagttctggctctggacgctcacggagtacgaccgggtcatcttcctggacgccgacctgctggtgcagcgccccatggagcctctgttcgcgatgcccgaggtgagcgccacggggaaccacggcgcctacttcaactcgggcgtcatggtcgtggagccctgcaactgcacgttccgcctgctggcggaccacgtcggcgacatcgattcgtacaacggcggcgaccaggggtacctcaacgaggtgttctcgtggtggcaccgcctgccgtcgcacgccaactacatgaagcacttctgggagggggacacggaggagcgcgccgcggccaagcgccgcgtgctggcggccgacccgcccatcgcgctcgccgtgcacttcgtcggcctcaaaccctggttctgcttccgggactacgactgcaactggaacgtgccggcgctgcgccagttcgccagcgacgaggcgcacgcgcgctggtggaaggtgcacgacgccatgccgcggcgcctccaggggttctgcctgctggacgagcggcagaaggcgctgctgtggtgggacgtcgcgcgggcgagggaggccaacttctccgacgcccactggagcgtccggatcgccgacccacgccggagtatctgcgccggcggcgacgccgaggcctgccgcgagagggagatcgccggccgccgggtggaagggaaccggatcaccacgtcgtacgccaagctaattgacaatttctgaattcagacggcagtaatagcgtcatattcatcccggccgtcggcggctggtcatccaacggcgc

SEQ ID NO: 67 - DNA, 1956 bp, Zea mays
atgaggggtttcgtcgcttgcgccgttgcagagaaacgccaccgcttggacaggacattatttagcggtttgagcaagaaggactgcatcgggaggtactacgccaaggatgccaccaagtacaggccgttcagcgcgctgctgcccgaggggggctggagcggcaaggtgctgtacgtcaagctcgtgctcgtcctcctcatgtgcggctccttcatgggcctcctcaactcgccgtccatccacctcgcggacgaacgtcaccaccacgcgcggactgcagaggcgtggaatgcgagctggacgtcgcaccccgacgcggcgaactccgggtacgcgtcgagcctgaggatcgactggtcgcaggtccagacagcggcgaagcaggtcgctccgccggcgggcggcggcgccacgcgtgtggcgctgctcaacttcgacgacggcgaggtccaggagtggaaggcgcggatgccgcacacggacgcctccacggtgcgcctggaccacgtcgggagcgacgtcacctgggaccacctgtacccggagtggatcgacgaggaggagcactacggcgcgccggcgtgcccggacctgccggagcctaaggtggccaaggaggaggaggcgtacgacgtcgtcgcggtcaagctgccttgcgggcgcgcggcgagctggtccaaggacgtggcgcgcctgcacctgcagctggccgcggcgcgtctcgcggccgcgcgcgcgccccgcggcggcggcgggcaggcggcgcacgtgctcgtggtcagccggtgcttcccgacgcccaacctgttcaggtgccgggacgaggtggcgcgccacggcgacgtgtggctgtacaggccggacgtgggcgacctcacccggaagctggagctccccgtggggtcctgcaagctggccatgccatccaaagcgctaggcgaacattacgcgtcggcggcgccgcagcgcgaggcgtacgccacgatcctccactcggagcagctgtacgcgtgcggcgccgtcacggcggcgcggagcatccggatggcggggtccgggcgggacatggtggccctggtggacgagacgatcagcgcgcggcaccgcgccgccctggaggcggccgggtggaaggtgcggacgatccggcgcatccgcaacccgcgggcgtcgcgggacgcgtacaacgagtggaactacagcaagttctggctctggacgctcacggagtacgaccgggtcatcttcctggacgccgacctgctggtgcagcgccccatggagcctctgttcgcgatgcccgaggtgagcgccacggggaaccacggcgcctacttcaactcgggcgtcatggtcgtggagccctgcaactgcacgttccgcctgctggcggaccacgtcggcgacatcgattcgtacaacggcggcgaccaggggtacctcaacgaggtgttctcgtggtggcaccgcctgccgtcgcacgccaactacatgaagcacttctgggagggggacacggaggagcgcgccgcggccaagcgccgcgtgctggcggccgacccgcccatcgcgctcgccgtgcacttcgtcggcctcaaaccctggttctgcttccgggactacgactgcaactggaacgtgccggcgctgcgccagttcgccagcgacgaggcgcacgcgcgctggtggaaggtgcacgacgccatgccgcggcgcctccaggggttctgcctgctggacgagcggcagaaggcgctgctgtggtgggacgtcgcgcgggcgagggaggccaacttctccgacgcccactggagcgtccggatcgccgacccacgccggagtatctgcgccggcggcgacgccgaggcctgccgcgagagggagatcgccggccgccgggtggaagggaaccggatcaccacgtcgtacgccaagctaattgacaatttctga

SEQ ID NO: 68 - protein, 651 aa, Zea mays
MRGFVACAVAEKRHRLDRTLFSGLSKKDCIGRYYAKDATKYRPFSALLPEGGWSGKVLYVKLVLVLLMCGSFMGLLNSPSIHLADERHHHARTAEAWNASWTSHPDAANSGYASSLRIDWSQVQTAAKQVAPPAGGGATRVALLNFDDGEVQEWKARMPHTDASTVRLDHVGSDVTWDHLYPEWIDEEEHYGAPACPDLPEPKVAKEEEAYDVVAVKLPCGRAASWSKDVARLHLQLAAARLAAARAPRGGGGQAAHVLVVSRCFPTPNLFRCRDEVARHGDVWLYRPDVGDLTRKLELPVGSCKLAMPSKALGEHYASAAPQREAYATILHSEQLYACGAVTAARSIRMAGSGRDMVALVDETISARHRAALEAAGWKVRTIRRIRNPRASRDAYNEWNYSKFWLWTLTEYDRVIFLDADLLVQRPMEPLFAMPEVSATGNHGAYFNSGVMVVEPCNCTFRLLADHVGDIDSYNGGDQGYLNEVFSWWHRLPSHANYMKHFWEGDTEERAAAKRRVLAADPPIALAVHFVGLKPWFCFRDYDCNWNVPALRQFASDEAHARWWKVHDAMPRRLQGFCLLDERQKALLWWDVARAREANFSDAHWSVRIADPRRSICAGGDAEACREREIAGRRVEGNRITTSYAKLIDNF

SEQ ID NO: 69 - protein, 585 aa, artificial sequence
  consensus
MGLEARYRPGAEDTKRRKSKSFKEEFDVVLEKSGCKFRSLLLLFAIMSAAFLTLLYTPSVYEHQLQSSSRVNGWIWDKSSDPRYISSASIQWEDVYKSLNLVGLSVGLLNFNTEFAWTMLPSDSVIRLEHAESITWQTLYPEWIDEEEETEIPSCPSLPDPFRAFDVVAVKLPCSRVAGWSRDVARLHLQLSAAKLAAATAKGNGIHVLFVTDCFPIPNLFCKDLVKREGAWLYKPDVKALKEKLRLPVGSCELAVPLAKARLYTVDRRREAYATILHSASEYVCGAITAAQSIRQAGSTRDLVILVDDTISDHHRKGLEAAGWKVRIIQRIRNPKAERDAYNEWNYSKFRLWQLTDYDKVIFIDADLLILRNIDFLFAMPEISATGNNATLFNSGVMVIEPSNCTFLLMEHIEITSYNGGDQGYLNEIFTWWHRIPKHMNFLKHFWEGDEDEVKAKKTRLFGADPPILYVLHYLGKPWLCFRDYDCNWNVEILREFASDVAHARWWKVHDKMPRKLQSYCLLRSRKALEWERRQAEKANFTDGHWKINVTDPRLKTCFEKFCFWESMLWHWGENNSTSSAVATS

SEQ ID NO: 70 - protein, 655 aa, Arabidopsis thaliana
MANSPAAPAPTTTTGGDSRRRLSASIEAICKRRFRRNSKGGGRSDMVKPFNIINFSTQDKNSSCCCFTKFQIVKLLLFILLSATLFTIIYSPEAYHHSLSHSSSRRQDPRYFSDLDINWDDVTKTLENIEEGRTIGVLNFDSNEIQRWREVSKSKDNGDEEKVVVLNLDYADKNVTWDALYPEWIDEEQETEVPVCPNIPNIKVPTRRLDLIVVKLPCRKEGNWSRDVGRLHLQLAAATVAASAKGFFRGHVFFVSRCFPIPNLFRCKDLVSRRGDVWLYKPNLDTLRDKLQLPVGSCELSLPLGIQDRPSLGNPKREAYATILHSAHVYVCGAIAAAQSIRQSGSTRDLVILVDDNISGYHRSGLEAAGWQIRTIQRIRNPKAEKDAYNEWNYSKFRLWQLTDYDKIIFIDADLLILRNIDFLFSMPEISATGNNGTLFNSGVMVIEPCNCTFQLLMEHINEIESYNGGDQGYLNEVFTWWHRIPKHMNFLKHFWIGDEDDAKRKKTELFGAEPPVLYVLHYLGMKPWLCYRDYDCNFNSDIFVEFATDIAHRKWWMVHDAMPQELHQFCYLRSKQKAQLEYDRRQAEAANYADGHWKIRVKDPRFKICIDKLCNWKSMLRHWGESNWTDYESFVPTPPAITVDRRSSLPGHNL

SEQ ID NO: 71 - protein, 596 aa, Arabidopsis thaliana
MTIMTMIMKMAPSKSALIRFNLVLLGFSFLLYTAIFFHPSSSVYFSSGASFVGCSFRDCTPKVVRGVKMQELVEENEINKKDLLTASNQTKLEAPSFMEEILTRGLGKTKIGMVNMEECDLTNWKRYGETVHIHFERVSKLFKWQDLFPEWIDEEEETEVPTCPEIPMPDFESLEKLDLVVVKLPCNYPEEGWRREVLRLQVNLVAANLAAKKGKTDWRWKSKVLFWSKCQPMIEIFRCDDLEKREADWWLYRPEVVRLQQRLSLPVGSCNLALPLWAPQGVDKVYDLTKIEAETKRPKREAYVTVLHSSESYVCGAITLAQSLLQTNTKRDLILLHDDSISITKLRALAAAGWKLRRIIRIRNPLAEKDSYNEYNYSKFRLWQLTDYDKVIFIDADIIVLRNLDLLFHFPQMSATGNDVWIYNSGIMVIEPSNCTFTTIMSQRSEIVSYNGGDQGYLNEIFVWWHRLPRRVNFLKNFWSNTTKERNIKNNLFAAEPPQVYAVHYLGWKPWLCYRDYDCNYDVDEQLVYASDAAHVRWWKVHDSMDDALQKFCRLTKKRRTEINWERRKARLRGSTDYHWKINVTDPRRRRSYLIG